(19) United States
(12) Patent Application Publication
(10) Pub. No.: US 2022/0332821 A1
(43) Pub. Date: Oct. 20, 2022

(54) DOSING REGIMEN AND COMBINATION THERAPIES FOR MULTISPECIFIC ANTIBODIES TARGETING B-CELL MATURATION ANTIGEN

(71) Applicant: NOVARTIS AG, Basel (CH)

(72) Inventors: Kimberly AARDALEN, Norwell, MA (US); Aida ABUJOUB, Winchester, MA (US); John BLANKENSHIP, Acton, MA (US); Anuradha CONNOR, Cambridge, MA (US); Mirek DOSTALEK, Binningen (CH); Tony FLEMING, Stow, MA (US); Brian HOLMBERG, Somerville, MA (US); Connie HONG, Somerville, MA (US); Lu HUANG, West Roxbury, MA (US); Haihui LU, Winchester, MA (US); K. Gary J. VANASSE, Wellesley, MA (US)

(21) Appl. No.: 17/620,987

(22) PCT Filed: Jun. 22, 2020
(86) PCT No.: PCT/IB2020/055872

§ 371 Date: Dec. 20, 2021

Related U.S. Application Data
(60) Provisional application No. 62/865,381, filed on Jun. 24, 2019.
(60) Provisional application No. 62/940,589, filed on Nov. 26, 2019.
(60) Provisional application No. 62/967,401, filed on Jan. 29, 2020.
(60) Provisional application No. 63/034,106, filed on Jun. 3, 2020.

(51) Int. Cl. C07K 16/28; A61P 35/00; A61K 9/00; A61K 31/167; A61K 31/417; A61K 31/56

(52) U.S. Cl. CPC C07K 16/2809; A61P 35/00; A61K 9/0019; A61K 31/167; A61K 31/417; A61K 31/56

(57) ABSTRACT

The present disclosure relates to dosing regimens, formulations, and combinations comprising a multispecific antibody having at least binding specificity towards B cell maturation antigen (BCMA) and a T-cell engaging arm; and methods of using such multispecific antibodies in the treatment or prevention of disease, such as, cancer.

### 1. SEQUENCE LISTING

The instant application contains a Sequence Listing which has been submitted electronically in ASCII format and is hereby incorporated by reference in its entirety. The ASCII copy, created on May 14, 2020, is named NOV-011USP4_SL.txt and is 15,390 bytes in size.

### 2. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

### 3. BACKGROUND

BCMA is a tumor necrosis family receptor (TNFR) member expressed on cells of the B-cell lineage. BCMA expression is the highest on terminally differentiated B cells that assume the long lived plasma cell fate, including plasma cells, plasmablasts and a subpopulation of activated B cells and memory B cells. BCMA is involved in mediating the survival of plasma cells for maintaining long-term humoral immunity. The expression of BCMA has been linked to a number of cancers, autoimmune disorders, and infectious diseases. Cancers with increased expression of BCMA include some hematological cancers, such as multiple myeloma, Hodgkin's and non-Hodgkin's lymphoma, various leukemias, and glioblastoma.

Various BCMA binding molecules are in clinical development, including BCMA antibody-drug conjugates such as GSK2857916 (GlaxoSmithkline) and bispecific BCMA binding molecules targeting BMCA and CD3 such as PF06863135 (Pfizer), EM 901 (EngMab), JNJ-64007957 (Janssen), and AMG 420 (Amgen). See, Cho et al., 2018, Front Immunol. 9:1821; WO 2016/0166629.

One of the primary safety concerns of any antibody-based drugs, including CD3 bispecific molecules, is its potential to induce life-threatening side effects such as cytokine release syndrome (“CRS”). See, Shimabukuro-Vornhagen et al., 2018, J. Immunother Cancer. 6:56.

Thus, there is an unmet medical need for polypeptides, e.g., antibodies and multispecific binding molecules, which bind BCMA, and which have an improved safety profile (e.g., decreasing cytokine release) while still retaining a high efficacy.

Further, there is an unmet medical need for the proper dosing of antibodies and multispecific binding molecules, which bind BCMA, in order to reduce the chances of producing unwanted side effects, including CRS.

### 4. SUMMARY

Disclosed herein, inter alia, are methods of using, and formulations, combinations, and compositions comprising a B cell maturation antigen (BCMA) binding molecule (e.g., a multispecific antibody, which can be an immunoglobulin-based multispecific binding molecule (MBM) described herein), more particularly a BCMA binding molecule that has the ability to target BCMA expressing cells and also the ability to engage a T-cell (e.g., by having a CD3 binding arm). The methods, formulations, combinations and compositions are exemplified by a BCMA binding molecule referred to herein as BSBM3. Thus, references to a “BCMA binding molecule” also apply to BSBM3.

Disclosed herein is a method of treating or preventing cancer (e.g., preventing relapse or recurrence of a cancer) comprising administering a BCMA binding molecule to a subject at a dose of about 0.25 μg/kg to about 1200 μg/kg (e.g., 1 μg/kg to about 1000 μg/kg).

The BCMA binding molecule can be administered at varying doses. For example, in one embodiment the BCMA binding molecule is administered to the subject at a dose of about 0.5 μg/kg to about 20 μg/kg. In one embodiment, the BCMA binding molecule is administered to the subject at a dose of about 0.5 μg/kg to 10 μg/kg. In one embodiment, the BCMA binding molecule is administered to the subject at a dose of about 1 μg/kg to 10 μg/kg. In one embodiment, the BCMA binding molecule is administered to the subject at a dose of about 5 μg/kg to 10 μg/kg. In one embodiment, the BCMA binding molecule is administered to the subject at a dose of about 1 μg/kg. In one embodiment, the BCMA binding molecule is administered to the subject at a dose of about 3 μg/kg. In one embodiment, the BCMA binding molecule is administered to the subject at a dose of about 6 μg/kg. In one embodiment, the BCMA binding molecule is administered to the subject at a dose of about 10 μg/kg. In one embodiment, the BCMA binding molecule is administered to the subject at a dose of about 10 μg/kg to 20 μg/kg. In one embodiment, the BCMA binding molecule is administered to the subject at a dose of about 10 μg/kg to 15 μg/kg. In one embodiment, the BCMA binding molecule is administered to the subject at a dose of about 12 μg/kg. In one embodiment, the BCMA binding molecule is administered to the subject at a dose of about 20 μg/kg to about 40 μg/kg. In one embodiment, the BCMA binding molecule is administered to the subject at a dose of about 20 μg/kg to about 30 μg/kg. In one embodiment, the BCMA binding molecule is administered to the subject at a dose of about 24 μg/kg. In one embodiment, the BCMA binding molecule is administered to the subject at a dose of about 30 μg/kg. In one embodiment, the BCMA binding molecule is administered to the subject at a dose of about 40 μg/kg to about 80 μg/kg. In one embodiment, the BCMA binding molecule is administered to the subject at a dose of about 40 μg/kg to about 60 μg/kg. In one embodiment, the BCMA binding molecule is administered to the subject at a dose of about 48 μg/kg. In one embodiment, the BCMA binding molecule is administered to the subject at a dose of about 80 μg/kg to about 120 μg/kg. In one embodiment, the BCMA binding molecule is administered to the subject at a dose of about 80 μg/kg to about 100 μg/kg. In one embodiment, the BCMA binding molecule is administered to the subject at a dose of about 96 μg/kg. In one embodiment, the BCMA binding molecule is administered to the subject at a dose of about 100 μg/kg. In one embodiment, the BCMA binding molecule is administered to the subject at a dose of about 100 μg/kg to about 200 μg/kg. In one embodiment, the BCMA binding molecule is administered to the subject at a dose of about 150 μg/kg to about 200 μg/kg. In one embodiment, the BCMA binding molecule is administered to the subject at a dose of about 192 μg/kg. In one embodiment, the BCMA binding molecule is administered to the subject at a dose of about 150 μg/kg to about 250 μg/kg. In one embodiment, the BCMA binding molecule is administered to the subject at a dose of about 200 μg/kg. In one embodiment, the BCMA binding molecule is administered to the subject at a dose of about 300 μg/kg to about 500 μg/kg. In one embodiment, the BCMA binding molecule is administered to the subject at a dose of about 384 μg/kg. In one embodiment, the BCMA binding molecule is administered to the subject at a dose of about 400 μg/kg. In one embodiment, the BCMA binding molecule is administered to the subject at a dose of about 500 μg/kg to about 700 μg/kg. In one embodiment, the BCMA binding molecule is administered to the subject at a dose of about 600 μg/kg.

The BCMA binding molecule is can be administered to the subject in any effective way. In some embodiments, the BCMA binding molecule is administered to the subject intravenously.

If the BCMA binding molecule can be administered to the subject intravenously, the BCMA binding molecule is administered over a certain span of time. For example, in one embodiment, the BCMA binding molecule is can be administered over a 2 hour span.

The BCMA binding molecule can also be administered to the subject one or more times over the course of time. For example, in one embodiment, the BCMA binding molecule can be administered to the subject, once a week for four weeks.

The BCMA binding molecule can also be administered as a priming dose. This priming dose can be administered prior to the beginning of treatment with a treatment dose (e.g., a therapeutic dose that is therapeutically effective). A priming dose can be a dose that is equal to or less than a subsequently administered treatment dose. In some embodiments, the BCMA binding molecule is administered as a priming dose at a dose that is lower than the first treatment dose. A priming dose can be administered in a single administration, or split among two or more administrations. In some embodiments, a priming dose is split into two administrations given on two consecutive days. In some embodiments, one third of a priming dose is administered to a subject on one day, and two thirds of the priming dose is administered to the subject the next day.

The BCMA binding molecule can also be administered along with a side effect reducing agent (e.g., acetaminophen and/or diphenhydramine). In one embodiment, the side effect reducing agent can be given at the same time as the BCMA binding molecule. In one embodiment, the side effect reducing agent can be given prior to the BCMA binding molecule. In one embodiment, the side effect reducing agent can be given after the BCMA binding molecule.

The side effect reducing agent can in some cases reduce the onset or severity of cytokine release syndrome (CRS). In one embodiment, the side effect reducing agent is a glucocorticoid. In one embodiment, the glucocorticoid is methylprednisolone. In one embodiment, the methylprednisolone is given to the subject at a dose of at least 2 mg/kg. In one embodiment, the side effect reducing agent is paracetamol, acetaminophen, antihistamines, steroids, anti-T cell directed therapy, or any combination thereof. In another embodiment, the side effect reducing agent is an anti-T cell directed therapy that is tocilizumab, canakinumab, or any combination thereof.

The subject who has received or will receive the BCMA binding molecule, can also be administered a second therapeutic agent. In some embodiments, the subject can receive one or more of the second therapeutic agents. In one embodiment, the BCMA binding molecule and the second therapeutic agent are administered simultaneously, separately, or over a period of time.

In one embodiment, the second therapeutic agent is a gamma secretase inhibitor (GSI). In one embodiment, the GSI is LY-450139, PF-5212362, BMS-708163, MK-0752, ELN-318463, BMS-299897, LY-411575, DAPT, AL-101 (BMS-906024), AL-102 (BMS-986115), PF-3084014, RO4929097, or LY3039478. In one embodiment, the GSI is administered orally. In one embodiment, the GSI is administered prior to administration of the BCMA binding molecule.

In one embodiment, the second therapeutic agent is an immunomodulator. In one embodiment, the second therapeutic agent is an immune checkpoint inhibitor. In one embodiment, the second therapeutic agent is a TIM-3 inhibitor. In one embodiment, the TIM-3 inhibitor is MBG453. In one embodiment, the second therapeutic agent is a LAG-3 inhibitor. In one embodiment, the LAG-3 inhibitor is LAG525. In one embodiment, the second therapeutic agent is a PD-1 inhibitor. In one embodiment, the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224. In one embodiment, the PD-1 inhibitor is PDR001. In one embodiment, the PD-1 inhibitor is administered at a dose of about 100 mg once every four weeks, or about 200 mg once every four weeks, or about 300 mg once every four weeks, or about 400 mg once every four weeks, or about 500 mg once every four weeks. In one embodiment, the PD-1 inhibitor is administered at a dose of about 400 mg once every four weeks.

The second therapeutic agent can be administered in any effective way. For example, the second therapeutic agent can be administered orally. In another embodiment, the second therapeutic agent can be administered intravenously.

The BCMA binding molecule and/or the one or more second therapeutic agents can prevent or treat cancer. In one embodiment, the cancer is a blood cancer. In one embodiment, the blood cancer is multiple myeloma.

In some embodiments, the subject has previously been treated for cancer. In one embodiment, the subject has relapsed and/or refractory multiple myeloma. In one embodiment, the subject has been previous treated with at least two prior treatment regimens. In one embodiment, the prior treatment regimens did not comprise a multispecific antibody. In one embodiment, the prior treatment regimens included an immunomodulatory drug (IMiD), a proteasome inhibitor, an anti-CD38 inhibitor, or any combination thereof. In one embodiment, the prior treatment regimens included an IMiD that was lenalidomide, pomalidomide, or both. In one embodiment, the prior treatment regimens included a proteasome inhibitor that was bortezomib, carfilzomib, or both. In one embodiment, the prior treatment regimens included an anti-CD38 inhibitor that was an anti-CD38 antibody. In one embodiment, the anti-CD38 antibody was daratumumab. In one embodiment, the prior treatment regimens included an autologous bone marrow transplant, a BCMA CAR-T, a BCMA antibody-drug conjugate, or any combination thereof.

The subject that is treated with the BCMA binding molecule can have (a) a serum M-protein greater than equal to 1.0 g/dL; (b) a urine M-protein greater than equal to 200 mg/24 hours; (c) a serum free light chain (sFLC) greater than 100 mg/L of involved FLC; or (d) any combination thereof.

In some embodiments, the subject that is treated with the BCMA binding molecule is not eligible for treatment with other anti-cancer regimens known to provide clinical benefit.

The subject that is treated with the BCMA binding molecule can include a subject that (a) does not have a history of severe hypersensitivity reactions to the BCMA binding molecule; (b) does not have a history of toxicity to prior BCMA targeted agents; (c) does not have any other malignant disease other than cancer being treated and/or prevented; (d) does not have any active, known or suspected autoimmune disease; (e) is not currently receiving treatment with a prohibited medication that cannot be discontinued at least one week prior to the start of treatment with the BCMA binding molecule; (f) is not infected with human immunodeficiency virus (HIV), active hepatitis B virus (HBV), or hepatitis C virus (HCV); (g) does not have impaired cardiac function or clinically significant cardiac disease including any of the following: (i) clinically significant and/or uncontrolled heart disease such as congestive heart failure requiring treatment (NYHA Grade≥2), uncontrolled hypertension or clinically significant arrhythmia; (ii) QTcF>470 msec on screening ECG or congenital long QT syndrome; or (iii) acute myocardial infarction or unstable angina pectoris<3 months prior to beginning of treatment with the BCMA binding molecule; (h) has not had radiotherapy within 14 days before the first dose of the BCMA binding molecule except for localized radiation therapy for lytic bone lesions or phasmacytomas; (i) has not had a major surgery within 2 weeks before the first dose of the BCMA binding molecule; (j) has not used systemic chronic steroid therapy (≤10 mg/day of prednisone or equivalent), or any immunosuppressive therapy within 7 days of first dose of the BCMA binding molecule; (k) does not receive systemic treatment with any immunosuppressive medication; (l) does not have Grade≥2 neuropathy, or residual toxic effects from previous therapy that have not resolved to Grade≤1 or baseline; (m) does not have plasma cell leukemia or other plasmacytoid disorder other than multiple myeloma; (n) does not have any of the following clinical laboratory results: (i) absolute neutrophil count (ANC)<1,000/mm3 without growth factor support within 7 days prior to the start of treatment; (ii) platelet count<75,000 mm3 without transfusion support within 7 days prior to the start of treatment; (iii) bilirubin>1.5 times the upper limit of the normal range (ULN); (iv) aspartate aminotransferase (AST) or alanine aminotransferase (ALT)>3 times the ULN; or (v) calculated creatinine clearance<30 ml/min according to Cockcroft-Gault equation; (o) does not have an active infection requiring systemic therapy or other severe infection within 2 weeks before the first dose of the BCMA binding molecule; (p) does not have POEMS syndrome (plasma cell dyscrasia with polyneuropathy, organomegaly, endocrinopathy, monoclonal protein, skin changes); (q) has not had prior allogeneic SCT at any time; (r) does not use of any live vaccines against infectious diseases (e.g. influenza, varicella, pneumococcus) within 4 weeks of the first dose of the BCMA binding molecule; (s) is not treated with cytotoxic or small molecule targeted antineoplastics, or any experimental therapy, within 14-days or 5 half-lives whichever is shorter before the first dose of the BCMA binding molecule; (t) has not had the initiation of hematopoietic colony-stimulating growth factors (e.g. G-CSF, M-CSF), thrombopoietin mimetics or erythroid stimulating agents≤2 weeks prior to start of treatment; (u) has not had intravenous IG infusions for infection prophylaxis within the last 28 days prior to treatment; (v) has not had active central nervous system (CNS) involvement by malignancy or presence of symptomatic CNS metastases, or CNS metastases that require local CNS-directed therapy (such as radiotherapy or surgery), or increasing doses of corticosteroids within the 2 weeks prior to the start of treatment; (w) does not have serious medical or psychiatric illness likely to interfere with the treatment; (x) is not a pregnant or nursing (lactating) woman; (y) is not a woman of child-bearing potential (defined as a woman physiologically capable of becoming pregnant) unless they are using effective methods (e.g., two) of contraception, including at least one highly effective method, during dosing and for six months after the last dose of the BCMA binding molecule is, wherein highly effective contraception methods include, but are not limited to, i) total abstinence, ii) female sterilization, iii) male sterilization, and (iv) use of oral, injected or implanted hormonal methods of contraception or placement of an intrauterine device (IUD) or intrauterine system (IUS), or other forms of hormonal contraception that have comparable efficacy (failure rate<1%), for example hormone vaginal ring or transdermal hormone contraception; and wherein other effective methods of contraception include barrier methods of contraception such as a condom or occlusive cap (diaphragm or cervical/vault caps) with spermicide (e.g., foam, gel, film, cream, or vaginal suppository); or (z) any combination thereof.

In some embodiments, the administering of the BCMA binding molecule continues until the subject experiences toxicity, has clinical evidence of disease progression by IMWG, and/or treatment is discontinued at the discretion of the treating physician.

Further disclosed herein is a combination therapy comprising the BCMA binding molecule and a second therapeutic agent. In some embodiments, the combination therapy can comprise two or more second therapeutic agents.

In some embodiments, the second therapeutic agent is a gamma secretase inhibitor (GSI). In some embodiments, the GSI is LY-450139, PF-5212362, BMS-708163, MK-0752, ELN-318463, BMS-299897, LY-411575, DAPT, AL-101 (BMS-906024), AL-102 (BMS-986115), PF-3084014, RO4929097, or LY3039478.

In some embodiments, the second therapeutic agent is an immunomodulator. In some embodiments, the second therapeutic agent is an immune checkpoint inhibitor. In some embodiments, the second therapeutic agent is a TIM-3 inhibitor. In some embodiments, the TIM-3 inhibitor is MBG453. In some embodiments, the second therapeutic agent is a LAG-3 inhibitor. In some embodiments, the LAG-3 inhibitor is LAG525. In some embodiments, the second therapeutic agent is a PD-1 inhibitor. In some embodiments, the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

In some embodiments, the combination comprises about 100 mg, or about 200 mg, or about 300 mg, or about 400 mg, or about 500 mg of the second therapeutic agent. In some embodiments, the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

Also described herein is a combination therapy as disclosed for use in the treatment of cancer. In some embodiments, the combination therapy is for use in the prevention of cancer.

In some embodiments, the disclosed here in the use of the combination therapy as described, for the manufacture of a medicament for treating or preventing cancer. In some embodiments, the use is for treatment of cancer. In some embodiments, the use is for the prevention of cancer.

In some embodiments, the cancer is a blood cancer. In some embodiments, the blood cancer is multiple myeloma.

Further described herein is a pharmaceutical composition comprising (a) a BCMA binding molecule; (b) histidine; (c) sucrose; and (d) PS20.

In some embodiments, the composition is a liquid. In some embodiments, the histidine concentration is 20 mM. In some embodiments, the sucrose concentration is 240 mM. In some embodiments, the PS20 concentration is 0.04%. In some embodiments, the pH is about 5.5±0.3.

Also described herein is a vial comprising (a) 10 mg/mL of a BCMA binding molecule; (b) 20 mM histidine; (c) 240 mM sucrose; (d) 0.04% PS20; and (e) a pH of about 5.5±0.3.

### 5. BRIEF DESCRIPTION OF THE FIGURES

FIG. 1: Format of the BCMA binding molecule designated as BSBM3.

FIGS. 2A-2C show BSBM3 mediated T cell proliferation, cytokine production and specific lysis of KMS11 myeloma cells via RTCC. Healthy donor T cells were co-cultured with KMS11 cells over-expressing luciferase at a 1:1 ratio in the presence of BSBM3 or non-targeting (NT) control antibody at the indicated concentrations. FIG. 2A shows levels of IFNγ and TNFα as measured by MSD assay with cell culture supernatants that were collected at 24 hr. FIG. 2B shows T cells counts as determined by CD3⁺ event counts using flow cytometry and normalized to counting beads controls after 4 days in coculture. FIG. 2C shows % RTCC (% lysis of KMS11 cells) as determined at 72 hr by the reduction in luciferase activity compared to KMS11 cells alone. Mean values+/−SEM are shown from three individual healthy donor T cells, each with three independent experiments (9 biological replicates total).

FIG. 3 shows that RTCC assay represents the most sensitive in vitro functional assays. EC30 values for BSBM3 were plotted for three different types of in vitro functional assays, RTCC, T cell proliferation and cytokine production (as shown in FIG. 2). Each data point represents one of nine biological replicates (T cells from three healthy donors were tested individually, each in three independent experiments).

FIG. 4 shows that soluble BCMA decreases the activity of BSBM3 in RTCC assay. The EC30 values for BSBM3 in RTCC assays with added soluble BCMA as indicated are shown. Each data point represents one of nine biological replicates (T cells from three healthy donor T cells were tested individually, each in three independent experiments).

FIG. 5 shows the anti-tumor activity of BSBM3 on KMS11 xenograft in a human PBMC adoptive transfer mouse model. NSG mice were inoculated with KMS11 cells via tail vein injection on Day 0 (D0), adoptively transferred with PBMCs on D7, and treated on D15 with the following doses of BSBM3: 0.03 mg/kg (triangle), 0.3 mg/kg (circle) or 3.0 mg/kg (diamond). For controls: tumor bearing mice without human PBMCs (increasing circles), tumor-bearing mice with human PBMCs but no Ab treatment (squares). The result from one representative experiment is shown from three biological replicates. *p<0.05, Dunnett's multiple comparison test. Data are expressed as the geometric mean+/−SEM from 5 mice per group.

FIG. 6 shows the clinical trial study schema for BSBM3.

FIG. 7 shows the international staging system for the BSBM3 clinical trial.

FIG. 8 shows the effect of AL-102 on BCMA shedding and membrane BCMA expression levels in KMS11 cells. Soluble BCMA levels (ng/mL) from culture supernatants of KMS11 cells treated for 20 hours with a serial dilution of AL-102 are shown on the left Y-axis. Antibody binding capacity (ABC) of anti-BCMA antibody (clone 19F2) on the surface of these AL-102 treated KMS11 cells is shown on the right Y axis.

FIG. 9 shows BSBM3 EC50 (mean+/−SEM) with increasing concentrations of AL-102: 1000 nM serially diluted 5-fold across 8-points. T-cell donor n=3. AL-102 enhanced the RTCC potency of BSBM3 in a dose dependent manner.

### 6. DETAILED DESCRIPTION

While some embodiments have been/will be shown and described throughout, such embodiments are provided by way of example only. Numerous variations, changes, and substitutions will now occur to those skilled in the art without departing from the invention. It should be understood that various alternatives to the embodiments of the invention described herein will be employed in practicing the invention.

The present disclosure provides methods of treating and/or preventing a disease (e.g., cancer) comprising administering to a subject in need thereof a composition comprising a BCMA binding molecule, particularly the BCMA binding molecule designated as BSBM3. In some aspects, the methods further comprise administering one or more therapeutic agents, e.g., one or more anti-tumor agents. The disclosure further provides formulations, dosing, dosing regimens and schedules, biomarkers, pharmaceutical combinations, and other relevant clinical features.

According to the present disclosure, additional therapeutic agents that can be used in combination with a BCMA binding molecule such as BSBM3, but are not limited to, an inhibitor of an inhibitory molecule (e.g., a checkpoint inhibitor), an activator of a costimulatory molecule, a chemotherapeutic agent, a targeted anti-cancer therapy, an oncolytic drug, a cytotoxic agent, or any of the therapeutic agents disclosed herein. In some embodiments, the one or more therapeutic agents can be a PD-1 inhibitor, a LAG-3 inhibitor, a cytokine, an A2A antagonist, a GITR agonist, a TIM-3 inhibitor, a STING agonist, and a TLR7 agonist, for treating and/or preventing a patient/subject with cancer.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### 6.1. Definitions

As used herein, the following terms are intended to have the following meanings:

ADCC: By “ADCC” or “antibody dependent cell-mediated cytotoxicity” as used herein is meant the cell-mediated reaction where nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

ADCP: By “ADCP” or antibody dependent cell-mediated phagocytosis as used herein is meant the cell-mediated reaction where nonspecific phagocytic cells that express FcγRs recognize bound antibody on a target cell and subsequently cause phagocytosis of the target cell.

Additional Aslant: For convenience, an agent that is used in combination with an antigen-binding molecule of the disclosure is referred to herein as an “additional” agent.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. In a wild-type antibody, at the N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antibody Numbering System: In the present specification, the references to numbered amino acid residues in antibody domains are based on the EU numbering system unless otherwise specified. This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

Antigen-binding domain: The term “antigen-binding domain” or “ABD” refers to a portion of an antigen-binding molecule that has the ability to bind to an antigen non-covalently, reversibly and specifically. Exemplary ABDs include antigen-binding fragments and portions of both immunoglobulin and non-immunoglobulin based scaffolds that retain the ability of binding an antigen non-covalently, reversibly and specifically. As used herein, the term “antigen-binding domain” encompasses antibody fragments that retain the ability of binding an antigen non-covalently, reversibly and specifically.

Antigen-binding domain chain or ABD chain: Individual ABDs can exist as one (e.g., in the case of an scFv) polypeptide chain or form through the association of more than one polypeptide chains (e.g., in the case of a Fab). As used herein, the term “ABD chain” refers to all ora portion of an ABD that exists on a single polypeptide chain. The use of the term “ABD chain” is intended for convenience and descriptive purposes only and does not connote a particular configuration or method of production.

Antigen-binding fragment: The term “antigen-binding fragment” of an antibody refers to a portion of an antibody that retains has the ability to bind to an antigen non-covalently, reversibly and specifically.

Antigen-binding molecule: The term “antigen-binding molecule” refers to a molecule comprising one or more antigen-binding domains, for example an antibody. The antigen-binding molecule can comprise one or more polypeptide chains, e.g., one, two, three, four or more polypeptide chains. The polypeptide chains in an antigen-binding molecule can be associated with one another directly or indirectly (for example a first polypeptide chain can be associated with a second polypeptide chain which in turn can be associated with a third polypeptide chain to form an antigen-binding molecule in which the first and second polypeptide chains are directly associated with one another, the second and third polypeptide chains are directly associated with one another, and the first and third polypeptide chains are indirectly associated with one another through the second polypeptide chain).

Associated: The term “associated” in the context of domains or regions within an antigen-binding molecule refers to a functional relationship between two or more polypeptide chains and/or two or more portions of a single polypeptide chain. In particular, the term “associated” means that two or more polypeptides (or portions of a single polypeptide) are associated with one another, e.g., non-covalently through molecular interactions and/or covalently through one or more disulfide bridges or chemical cross-linkages, so as to produce a functional antigen-binding domain. Examples of associations that might be present in an antigen-binding molecule include (but are not limited to) associations between Fc regions in an Fc domain, associations between VH and VL regions in a Fab or Fv, and associations between CH1 and CL in a Fab.

B cell: As used herein, the term “B cell” refers to a cell of B cell lineage, which is a type of white blood cell of the lymphocyte subtype. Examples of B cells include plasmablasts, plasma cells, lymphoplasmacytoid cells, memory B cells, follicular B cells, marginal zone B cells, B-1 cells, B-2 cells, and regulatory B cells.

B cell malignancy: As used herein, a B cell malignancy refers to an uncontrolled proliferation of B cells. Examples of B cell malignancy include non-Hodgkin's lymphomas (NHL), Hodgkin's lymphomas, leukemia, and myeloma. For example, a B cell malignancy can be, but is not limited to, multiple myeloma, chronic lymphocytic leukemia (CLL)/small lymphocytic lymphoma (SLL), follicular lymphoma, mantle cell lymphoma (MCL), diffuse large B-cell lymphoma (DLBCL), marginal zone lymphomas, Burkitt lymphoma, lymphoplasmacytic lymphoma (Waldenstrom macroglobulinemia), hairy cell leukemia, primary central nervous system (CNS) lymphoma, primary mediastinal large B-cell lymphoma, mediastinal grey-zone lymphoma (MGZL), splenic marginal zone B-cell lymphoma, extranodal marginal zone B-cell lymphoma of MALT, nodal marginal zone B-cell lymphoma, and primary effusion lymphoma, and plasmacytic dendritic cell neoplasms.

BCMA: As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells and plasma cells. Its ligands include B-cell activating factor (BAFF) and a proliferation-inducing ligand (APRIL). The protein BCMA is encoded by the gene TNFRSF17. Exemplary BCMA sequences are available at the Uniprot database under accession number Q02223.

BCMA binding molecule: The term “BCMA binding molecule” refers to a molecule that specifically binds to BCMA, particularly human BCMA. Examples of BCMA binding molecules including multispecific binding molecules that comprise at least one ABD that binds to BCMA, e.g., multispecific antibodies, bispecific antibodies and other bispecific binding molecules. A particular BCMA binding molecule of the disclosure is referred to herein as BSBM3.

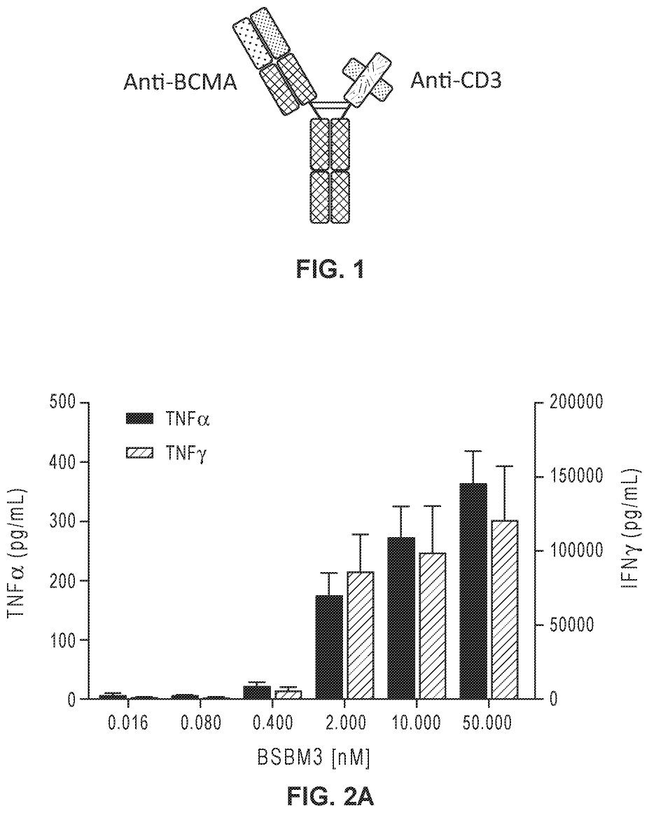

Bispecific binding molecule: The term “bispecific binding molecule” or “BBM” refers to a molecule that specifically binds to two antigens and comprises two or more ABDs. The BBMs of the disclosure comprise at least one antigen-binding domain which is specific for BCMA and at least one antigen-binding domain which is specific for a different antigen, e.g., component of a TCR complex. Representative BBMs are illustrated in FIG. 1B-1AG. BBMs can comprise one, two, three, four or even more polypeptide chains.

Bivalent: The term “bivalent” as used herein in the context of an antigen-binding molecule refers to an antigen-binding molecule that has two ABDs. The domains can be the same or different. Accordingly, a bivalent antigen-binding molecule can be monospecific or bispecific. Bivalent BBMs comprise an ABD that specifically binds to BCMA and another ABD that binds to another antigen, e.g., a component of the TCR complex.

BSBM3: BSMB3 refers to a BCMA binding molecule comprising (a) a first polypeptide whose amino acid sequence comprises the amino acid sequence of SEQ ID NO:1; (b) a second polypeptide whose amino acid sequence comprises the amino acid sequence of SEQ ID NO:2; and (c) a third polypeptide whose amino acid sequence comprises the amino acid sequence of SEQ ID NO:3. When co-expressed, the first, second and third polypeptide associate to form a binding molecule with the configuration shown in FIG. 1.

Cancer: The term “cancer” refers to a disease characterized by the uncontrolled (and often rapid) growth of aberrant cells. Cancer cells can spread locally or through the bloodstream and lymphatic system to other parts of the body. Examples of various cancers are described herein and include but are not limited to, leukemia, multiple myeloma, asymptomatic myeloma, Hodgkin's lymphoma and non-Hodgkin's lymphoma, e.g., any BCMA-positive cancers of any of the foregoing types. The term “cancerous B cell” refers to a B cell that is undergoing or has undergone uncontrolled proliferation

CD3: The term “CD3” or “cluster of differentiation 3” refers to the cluster of differentiation 3 co-receptor of the T cell receptor. CD3 helps in activation of both cytotoxic T-cell (e.g., CD8+ naïve T cells) and T helper cells (e.g., CD4+ naïve T cells) and is composed of four distinct chains: one CD3γ chain (e.g., Genbank Accession Numbers NM_000073 and MP_000064 (human)), one CD315 chain (e.g., Genbank Accession Numbers NM_000732, NM_001040651, NP_00732 and NP_001035741 (human)), and two CD3ε chains (e.g., Genbank Accession Numbers NM_000733 and NP_00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes. Unless expressly indicated otherwise, the reference to CD3 in the application can refer to the CD3 co-receptor, the CD3 co-receptor complex, or any polypeptide chain of the CD3 co-receptor complex.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any one of a number of well-known schemes, including those described by Kabat et al., 1991, “Sequences of Proteins of Immunological Interest,” 5th Ed. Public Health Service, National Institutes of Health, Bethesda, Md. (“Kabat” numbering scheme), Al-Lazikani et al., 1997, JMB 273,927-948 (“Chothia” numbering scheme), or a combination thereof, and ImMunoGenTics (IMGT) numbering (Lefranc, 1999, The Immunologist, 7:132-136; Lefranc et al., 2003, Dev. Comp. Immunol. 27, 55-77 (“IMGT” numbering scheme). In a combined Kabat and Chothia numbering scheme for a given CDR region (for example, HC CDR1, HC CDR2, HC CDR3, LC CDR1, LC CDR2 or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

For example, under Kabat, the CDR amino acid residues in the heavy chain variable domain (VH) are numbered 31-35 (CDR-H1) (e.g., insertion(s) after position 35), 50-65 (CDR-H2), and 95-102 (CDR-H3); and the CDR amino acid residues in the light chain variable domain (VL) are numbered 24-34 (CDR-L1) (e.g., insertion(s) after position 27), 50-56 (CDR-L2), and 89-97 (CDR-L3). As another example, under Chothia, the CDR amino acids in the VH are numbered 26-32 (CDR-H1) (e.g., insertion(s) after position 31), 52-56 (CDR-H2), and 95-102 (CDR-H3); and the amino acid residues in VL are numbered 26-32 (CDR-L1) (e.g., insertion(s) after position 30), 50-52 (CDR-L2), and 91-96 (CDR-L3). By combining the CDR definitions of both Kabat and Chothia, the CDRs comprise or consist of, e.g., amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align. Generally, unless specifically indicated, the antibody molecules can include any combination of one or more Kabat CDRs and/or Chothia CDRs.

Concurrently: The term “concurrently” is not limited to the administration of therapies (e.g., prophylactic or therapeutic agents) at exactly the same time, but rather it is meant that a pharmaceutical composition comprising an antigen-binding molecule is administered to a subject in a sequence and within a time interval such that the molecules can act together with the additional therapy(ies) to provide an increased benefit than if they were administered otherwise.

Conservative Sequence Modifications: The term “conservative sequence modifications” refers to amino acid modifications that do not significantly affect or alter the binding characteristics of a BCMA binding molecule ora component thereof (e.g., an ABD or an Fc region). Such conservative modifications include amino acid substitutions, additions and deletions. Modifications can be introduced into a BBM by standard techniques, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a BBM can be replaced with other amino acid residues from the same side chain family and the altered BBM can be tested for, e.g., binding to target molecules and/or effective heterodimerization and/or effector function.

Epitope: An epitope, or antigenic determinant, is a portion of an antigen recognized by an antibody or other antigen-binding moiety as described herein. An epitope can be linear or conformational.

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which can be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, ADCC, ADCP, release of inflammatory mediators, placental transfer and control of immunoglobulin production. An effector function of an antibody can be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but can alter the geometry of the interaction rendering the effector mechanism ineffective as in non-productive binding. It is further envisaged that an effector function can also be altered by modifying a site not directly involved in effector molecule binding, but otherwise involved in performance of the effector function.

Fab: By “Fab” or “Fab region” as used herein is meant a polypeptide region that comprises the VH, CH1, VL, and CL immunoglobulin domain. These terms can refer to this region in isolation, or this region in the context of an antigen-binding molecule.

Fab domains are formed by association of a CH1 domain attached to a VH domain with a CL domain attached to a VL domain. The VH domain is paired with the VL domain to constitute the Fv region, and the CH1 domain is paired with the CL domain to further stabilize the binding module. A disulfide bond between the two constant domains can further stabilize the Fab domain.

Fab regions can be produced by proteolytic cleavage of immunoglobulin molecules (e.g., using enzymes such as papain) or through recombinant expression. In native immunoglobulin molecules, Fabs are formed by association of two different polypeptide chains (e.g., VH-CH1 on one chain associates with VL-CL on the other chain). The Fab regions are typically expressed recombinantly, typically on two polypeptide chains, although single chain Fabs are also contemplated herein.

Fc region: The term “Fc region” or “Fc chain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc region” includes both amino acids 231-447 (CH2-CH3) or 216-447 (hinge-CH2-CH3), or fragments thereof. An “Fc fragment” in this context can contain fewer amino acids from either or both of the N- and C-termini but still retains the ability to form a dimer with another Fc region as can be detected using standard methods, generally based on size (e.g., non-denaturing chromatography, size exclusion chromatography). Human IgG Fc regions are of particular use in the present disclosure, and can be the Fc region from human IgG1, IgG2 or IgG4.

Fc domain: The term “Fc domain” refers to a pair of associated Fc regions. The two Fc regions dimerize to create the Fc domain. The two Fc regions within the Fc domain can be the same (such an Fc domain being referred to herein as an “Fc homodimer”) or different from one another (such an Fc domain being referred to herein as an “Fc heterodimer”).

Fv: The term “Fv”, “Fv fragment” or “Fv region” refer to a region that comprises the VL and VH domains of an antibody fragment in a tight, noncovalent association (a VH-VL dimer). It is in this configuration that the three CDRs of each variable domain interact to define a target binding site. Often, the six CDRs confer target binding specificity to an antigen-binding molecule. However, in some instances even a single variable domain (or half of an Fv comprising only three CDRs specific for a target) can have the ability to recognize and bind target. In a native immunoglobulin molecule, the VH and VL of an Fv are on separate polypeptide chains but can be engineered as a single chain Fv (scFv). The terms also include Fvs that are engineered by the introduction of disulfide bonds for further stability.

The reference to a VH-VL dimer herein is not intended to convey any particular configuration. For example, in scFvs, the VH can be N-terminal or C-terminal to the VL (with the VH and VL typically connected by a linker as discussed herein).

Half Antibody: The term “half antibody” refers to a molecule that comprises at least one ABD or ABD chain and can associate with another molecule comprising an ABD or ABD chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In an embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, where the VL and VH domains form an ABD. Yet another example of a half antibody is a polypeptide comprising an scFv domain, a CH2 domain and a CH3 domain.

A half antibody might include more than one ABD, for example a half-antibody comprising (in N- to C-terminal order) an scFv domain, a CH2 domain, a CH3 domain, and another scFv domain.

Half antibodies might also include an ABD chain that when associated with another ABD chain in another half antibody forms a complete ABD.

Thus, a BBM can comprise one, more typically two, or even more than two half antibodies, and a half antibody can comprise one or more ABDs or ABD chains.

In some BBMs, a first half antibody will associate, e.g., heterodimerize, with a second half antibody. In other BBMs, a first half antibody will be covalently linked to a second half antibody, for example through disulfide bridges or chemical crosslinking. In yet other BBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Hole: In the context of a knob-into-hole, a “hole” refers to at least one amino acid side chain which is recessed from the interface of a first Fc chain and is therefore positionable in a compensatory “knob” on the adjacent interfacing surface of a second Fc chain so as to stabilize the Fc heterodimer, and thereby favor Fc heterodimer formation over Fc homodimer formation, for example.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing an antigen-binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. Humanized antibodies are typically less immunogenic to humans, relative to non-humanized antibodies, and thus offer therapeutic benefits in certain situations. Humanized antibodies can be generated using known methods. See for example, Hwang et al., 2005, Methods 36:35; Queen et al., 1989, Proc. Natl. Acad. Sci. U.S.A. 86:10029-10033; Jones et al., 1986, Nature 321:522-25, 1986; Riechmann et al., 1988, Nature 332:323-27; Verhoeyen et a1.,1988, Science 239:1534-36; Orlandi et al., 1989, Proc. Natl. Acad. Sci. U.S.A. 86:3833-3837; U.S. Pat. Nos. 5,225,539; 5,530,101; 5,585,089; 5,693,761; 5,693,762; and 6,180,370; and WO 90/07861. See also the following review articles and references cited therein: Presta, 1992, Curr. Op. Struct. Biol. 2:593-596; Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, can be defined using well known numbering schemes, e.g., the Kabat numbering scheme, the Chothia numbering scheme, or any combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies can include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

In combination: Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons.

Knob: In the context of a knob-into-hole, a “knob” refers to at least one amino acid side chain which projects from the interface of a first Fc chain and is therefore positionable in a compensatory “hole” in the interface with a second Fc chain so as to stabilize the Fc heterodimer, and thereby favor Fc heterodimer formation over Fc homodimer formation, for example.

Knobs and holes (or knobs-into-holes): One mechanism for Fc heterodimerization is generally referred to in the art as “knobs and holes”, or “knob-in-holes”, or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; and U.S. Pat. No. 8,216,805. Knob-in-hole mutations can be combined with other strategies to improve heterodimerization.

Monoclonal Antibody: The term “monoclonal antibody” as used herein refers to polypeptides, including antibodies, antibody fragments, molecules (including BBMs), etc. that are derived from the same genetic source.

Monovalent: The term “monovalent” as used herein in the context of an antigen-binding molecule refers to an antigen-binding molecule that has a single antigen-binding domain.

Multispecific binding molecule: The term “multispecific binding molecule” or “MBM” refers to an antigen-binding molecule that specifically binds to at least two antigens and comprises two or more ABDs. The ABDs can each independently be an antibody fragment (e.g., scFv, Fab, nanobody), a ligand, or a non-antibody derived binder (e.g., fibronectin, Fynomer, DARPin).

Mutation or modification: In the context of the primary amino acid sequence of a polypeptide, the terms “modification” and “mutation” refer to an amino acid substitution, insertion, and/or deletion in the polypeptide sequence relative to a reference polypeptide. Additionally, the term “modification” further encompasses an alteration to an amino acid residue, for example by chemical conjugation (e.g., of a drug or polyethylene glycol moiety) or post-translational modification (e.g., glycosylation).

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions can be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of an antigen-binding molecule, separate ABMs (or chains of an ABM) can be operably linked through peptide linker sequences. In the context of a nucleic acid encoding a fusion protein, such as a polypeptide chain of an antigen-binding molecule, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide and Protein: The terms “polypeptide” and “protein” are used interchangeably herein to refer to a polymer of amino acid residues. The terms encompass amino acid polymers in which one or more amino acid residue is an artificial chemical mimetic of a corresponding naturally occurring amino acid, as well as to naturally occurring amino acid polymers and non-naturally occurring amino acid polymer. Additionally, the terms encompass amino acid polymers that are derivatized, for example, by synthetic derivatization of one or more side chains or termini, glycosylation, PEGylation, circular permutation, cyclization, linkers to other molecules, fusion to proteins or protein domains, and addition of peptide tags or labels.

Recognize: The term “recognize” as used herein refers to an ABD that finds and interacts (e.g., binds) with its epitope.

Single Chain Fab or scFab: The terms “single chain Fab” and “scFab” mean a polypeptide comprising an antibody heavy chain variable domain (VH), an antibody constant domain 1 (CH1), an antibody light chain variable domain (VL), an antibody light chain constant domain (CL) and a linker, such that the VH and VL are in association with one another and the CH1 and CL are in association with one another. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, e.g., between 32 and 50 amino acids. The single chain Fabs are stabilized via the natural disulfide bond between the CL domain and the CH1 domain.

Simultaneous or concurrent delivery: In some embodiments, the delivery of one treatment is still occurring when the delivery of a second begins, so that there is overlap in terms of administration. This is sometimes referred to herein as “simultaneous” or “concurrent delivery”. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Single Chain Fv or scFv: By “single chain Fv” or “scFv” herein is meant a variable heavy domain covalently attached to a variable light domain, generally using an ABD linker as discussed herein, to form a scFv or scFv domain. A scFv domain can be in either orientation from N- to C-terminus (VH-linker-VL or VL-linker-VH). For a review of scFv see Pluckthun in The Pharmacology of Monoclonal Antibodies, vol. 113, Rosenburg and Moore eds., (1994) Springer-Verlag, New York, pp. 269-315.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. An antigen-binding molecule or ABD of the disclosure typically has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater (and more typically at least 20-fold, at least 50-fold or at least 100-fold) than its affinity for binding to a non-specific antigen (e.g., HSA). Binding affinity can be measured using a Biacore, SPR or BLI assay.

The term “specifically binds” does not exclude cross-species reactivity. For example, an antigen-binding module (e.g., an antigen-binding fragment of an antibody) that “specifically binds” to an antigen from one species can also “specifically bind” to that antigen in one or more other species. Thus, such cross-species reactivity does not itself alter the classification of an antigen-binding module as a “specific” binder. In certain embodiments, an antigen-binding domain that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding domain does not have cross-species reactivity.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in some embodiments a BBM has 3, 4, 5, 6, 7, 8, 9, or 10 VH domains. The tandem of VH can be produced by joining the encoding nucleic acids of each VH domain in a desired order using recombinant methods with or without a linker that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in some embodiments a BBM has 3, 4, 5, 6, 7, 8, 9, or 10 VL domains. The tandem of VL can be produced by joining the encoding nucleic acids of each VL domain in a desired order using recombinant methods with or without a linker that enables them to be made as a single polypeptide chain. The N-terminus of the first VL domain in the tandem is defined as the N-terminus of the tandem, while the C-terminus of the last VL domain in the tandem is defined as the C-terminus of the tandem.

Target Antigen: By “target antigen” as used herein is meant the molecule that is bound non-covalently, reversibly and specifically by an antigen binding domain.

Therapeutically effective amount: A “therapeutically effective amount” refers to an amount effective, at dosages and for periods of time necessary, to achieve a desired therapeutic result.

Treat, Treatment, Treating: As used herein, the terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more antigen-binding molecules. In some embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Tumor: The term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

Variable region: By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. A “variable heavy domain” can pair with a “variable light domain” to form an antigen binding domain (“ABD”). In addition, each variable domain comprises three hypervariable regions (“complementary determining regions,” “CDRs”) (CDR-H1, CDR-H2, CDR-H3 for the variable heavy domain and CDR-L1, CDR-L2, CDR-L3 for the variable light domain) and four framework (FR) regions, arranged from amino-terminus to carboxy-terminus in the following order: FR1-CDR1-FR2-CDR2-FR3-CDR3-FR4.

Vector: The term “vector” is intended to refer to a polynucleotide molecule capable of transporting another polynucleotide to which it has been linked. One type of vector is a “plasmid”, which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

VH-VL or VH-VL Pair: In reference to a VH-VL pair, whether on the same polypeptide chain or on different polypeptide chains, the terms “VH-VL” and “VH-VL pair” are used for convenience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

### 6.2. BCMA Binding Molecules

The present disclosure provides therapeutic regimens and formulations of BCMA binding molecules. Preferred BCMA binding molecules are multispecific binding molecules, e.g., multispecific antibodies, more specifically bispecific binding molecules, e.g., bispecific antibodies, that bind to BCMA and CD3.

In a particularly preferred emboidments, the BCMA binding molecule is the molecule referred to herein as BSBM3. BSBM3 has a Fab domain targeting BCMA and a single-chain Fv (scFv) domain targeting CD3. BSBM3 is composed of three polypeptides which, when expressed in the same cell, form two half antibodies as shown in FIG. 1. The first half antibody, composed of a heavy chain polypeptide having the amino acid sequence of SEQ ID NO:1 associated with a light chain polypeptide having the amino acid sequence of SEQ ID NO:2, contains a Fab domain that binds to CD19. The second half antibody, composed of a heavy chain polypeptide having the amino acid sequence of SEQ ID NO:3, contains an scFv domain that binds to CD3. The Fc domain of BSBM3 contains substitutions that ablate binding to human Fcy receptors in order to reduce the risk of non-selective T cell activation via FcR (Fc receptor)-mediated crosslinking. Without being bound by theory, it is believed that the Fc domain confers IgG-like in vivo persistence due to unmodified FcRn (neonatal Fc receptor) affinity. It is also believed, without being bound by theory, that binding of multiple molecules of BSBM3 simultaneously with BCMA on multiple myeloma (MM) cells and the CD3 subunit of the T cell receptor (TCR) complex on T cells leads to TCR crosslinking and formation of a cytolytic immune synapse, resulting in activation of T cells and specific lysis of MM cells.

### 6.3. Pharmaceutical Compositions

The BCMA binding molecules of the disclosure can be formulated as pharmaceutical compositions comprising the BCMA binding molecules, for example containing one or more pharmaceutically acceptable excipients or carriers. To prepare pharmaceutical or sterile compositions comprising the BCMA binding molecules of the present disclosure a BCMA binding molecule preparation can be combined with one or more pharmaceutically acceptable excipient or carrier.

For example, formulations of BCMA binding molecules can be prepared by mixing BCMA binding molecules with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis et al. (eds.),1993, Pharmaceutical Dosage Forms: General Medications, Marcel Dekker, NY; Lieberman et al. (eds.), 1990, Pharmaceutical Dosage Forms: Tablets, Marcel Dekker, NY; Lieberman et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

For intravenous formulations, the BCMA binding molecule can be formulated with one or more excipients. In one embodiment, the BCMA binding molecule is formulated with an amino acid. In one embodiment, the BCMA binding molecule is formulated with a sugar. In one embodiment, the BCMA binding molecule is formulated with a surfactant. In one embodiment, the BCMA binding molecule is formulated with water. In some embodiments, the BCMA binding molecule can be formulated with one or more of an amino acid, a sugar, or a surfactant.

In some embodiments, the amino acid can be histidine. In some embodiments, the sugar can be sucrose. In some embodiments, the surfactant can be polysorbate, such as polysorbate 20 (“PS20”), also known as Tween 20.

Accordingly, the disclosure provides pharmaceutical compositions comprising a BCMA binding molecule and (a) an amino acid such as histidine; (b) a sugar such as sucrose; (c) a surfactant such as PS20; or (d) a combination of any two or all of the foregoing. For parenteral, e.g., intravenous, administration, the pharmaceutical composition can be a liquid pharmaceutical composition.

Suitable concentrations of histidine range from 10 mM to 50 mM. In an embodiment, the concentration of histidine is 20 mM.

Suitable concentrations of sucrose range from 150 mM to 300 mM. In an embodiment, the concentration of sucrose is 240 mM.

Suitable concentrations of PS20 range from 0.02% to 0.06%. In an embodiment, the concentration of PS20 is 0.04%.

The pharmaceutical composition can be lyophilized and reconstituted in a suitable volume of liquid to obtain a solution for administration containing one or more of histidine, sucrose and PS20, e.g., in the concentrations described above.

In some embodiments, the pH of the formulation can be acidic. For example, in one embodiment, the pH of the formulation can be about 5.0 to about 6.5. In one embodiment, the pH of the formation can be about 5.0 to about 6.0. In some embodiment, the pH of the formulation can be about 5.5.

A suitable pH range for a liquid pharmaceutical composition comprising a BCMA binding molecule for parenteral, e.g., intravenous, administration is acidic, e.g., about 5.0 to about 6.5. In certain aspects, the pH is about 5.0 to about 6.0 and in some embodiments the pH is about 5.5.

A suitable concentration range for the BCMA binding molecule is between 5 mg/mL and 20 mg/mL, and in an embodiment is 10 mg/mL.

Accordingly, in a particular embodiment, the disclosure provides a vial comprising (a) 10 mg/mL of BSBM3; (b) 20 mM histidine; (c) 240 mM sucrose; (d) 0.04% PS20; and (e) a pH of about 5.5±0.3.

### 7. DOSING

### 7.1. BCMA Binding Molecule Amount

BCMA binding molecules can be used for the prevention and/or treatment of cancer.

In some embodiments, the subject can be dosed with the BCMA binding molecule with from about 0.5 μg/kg. In some embodiments, the subject can be dosed with the BCMA binding molecule with from about 1 μg/kg. In some embodiments, the subject can be dosed with the BCMA binding molecule with from about 10 μg/kg. In some embodiments, the subject can be dosed with the BCMA binding molecule with from about 30 μg/kg. In some embodiments, the subject can be dosed with the BCMA binding molecule with from about 50 μg/kg. In some embodiments, the subject can be dosed with the BCMA binding molecule with from about 75 μg/kg. In some embodiments, the subject can be dosed with the BCMA binding molecule with from about 100 μg/kg. In some embodiments, the subject can be dosed with the BCMA binding molecule with from about 200 μg/kg. In some embodiments, the subject can be dosed with the BCMA binding molecule with from about 300 μg/kg. In some embodiments, the subject can be dosed with the BCMA binding molecule with from about 400 μg/kg. In some embodiments, the subject can be dosed with the BCMA binding molecule with from about 500 μg/kg. In some embodiments, the subject can be dosed with the BCMA binding molecule with from about 600 μg/kg. In some embodiments, the subject can be dosed with the BCMA binding molecule with from about 700 μg/kg. In some embodiments, the subject can be dosed with the BCMA binding molecule with from about 800 μg/kg. In some embodiments, the subject can be dosed with the BCMA binding molecule with from about 900 μg/kg. In some embodiments, the subject can be dosed with the BCMA binding molecule with from about 1000 μg/kg.

For example, in some embodiments, the subject can be given the BCMA binding molecule at a dose of from about 1 μg/kg to about 20 μg/kg. In some embodiments, the subject can be given the BCMA binding molecule at a dose of from about 20 μg/kg to about 40 μg/kg. In some embodiments, the subject can be given the BCMA binding molecule at a dose of from about 80 μg/kg to about 120 μg/kg. In some embodiments, the subject can be given the BCMA binding molecule at a dose of from about 150 μg/kg to about 250 μg/kg. In some embodiments, the subject can be given the BCMA binding molecule at a dose of from about 300 μg/kg to about 500 μg/kg. In some embodiments, the subject can be given the BCMA binding molecule at a dose of from about 500 μg/kg to about 700 μg/kg. In some embodiments, the subject can be given the BCMA binding molecule at a dose of from about 600 μg/kg to about 900 μg/kg.

In some embodiments, the subject can be dosed with the BCMA binding molecule with from about 1 μg/kg to about 1000 μg/kg. In some embodiments, the subject can be given the BCMA binding molecule at a dose of from about 10 μg/kg to about 900 μg/kg. In some embodiments, the subject can be given the BCMA binding molecule at a dose of from about 20 μg/kg to about 800 μg/kg. In some embodiments, the subject can be given the BCMA binding molecule at a dose of from about 30 μg/kg to about 700 μg/kg. In some embodiments, the subject can be given the BCMA binding molecule at a dose of from about 50 μg/kg to about 600 μg/kg. In some embodiments, the subject can be given the BCMA binding molecule at a dose of from about 75 μg/kg to about 500 μg/kg. In some embodiments, the subject can be given the BCMA binding molecule at a dose of from about 100 μg/kg to about 400 μg/kg. In some embodiments, the subject can be given the BCMA binding molecule at a dose of from about 150 μg/kg to about 300 μg/kg. In some embodiments, the subject can be given the BCMA binding molecule at a dose of from about 200 μg/kg to about 250 μg/kg.

Further, any of the dosing amounts disclosed throughout this disclosure can be used to dose the BCMA binding molecule, e.g., as a first or subsequent treatment dose.

In an embodiment, a treatment dose can be about 1 μg/kg to about 1200 μg/kg or about 50 μg to about 96 mg. In another embodiment, a treatment dose can be about 3 μg/kg to about 600 μg/kg or about 150 μg to about 48 mg. In another embodiment, a treatment dose can be about 5 μg/kg to about 100 μg/kg or about 150 μg to about 8 mg. In another embodiment, a treatment dose can be about 10 μg/kg to about 200 μg/kg or about 500 μg to about 16 mg. In another embodiment, a treatment dose can be about 50 μg/kg to about 400 μg/kg or about 2.5 mg to about 32 mg. In another embodiment, a treatment dose can be about 100 μg/kg to about 600 μg/kg or about 5 mg to about 96 mg. In another embodiment, a treatment dose can be about 1 μg/kg or about 50 μg to about 80 μg. In another embodiment, a treatment dose can be about 3 μg/kg. In another embodiment, a treatment dose can be about 150 μg to about 240 μg. In another embodiment, a treatment dose can be about 6 μg/kg or about 300 μg to about 480 μg. In another embodiment, a treatment dose can be about 12 μg/kg or about 600 μg to about 960 μg. In another embodiment, a treatment dose can be about 24 μg/kg or about 1.2 mg to about 1.92 mg. In another embodiment, a treatment dose can be about 48 μg/kg or about 2.4 mg to about 3.84 mg. In another embodiment, a treatment dose can be about 96 μg/kg or about 4.8 mg to about 7.68 mg. In another embodiment, a treatment dose can be about 192 μg/kg or about 9.6 mg to about 15.36 mg. In another embodiment, a treatment dose can be about 384 μg/kg or about 19.2 mg to about 30.72 mg. In another embodiment, a treatment dose can be about 600 μg/kg or about 30 mg to about 48 mg.

In some instances, a priming dose is needed or used. The priming dose can be any of the doses described herein, and in some embodiments is lower than the first treatment dose. For example, if the first treatment dosing amount of the BCMA binding molecule is 30 μg/kg, the priming dose can be given at any dose lower than 30 μg/kg. In this particular case, the priming dose can be given at a dose lower than 30 μg/kg, for example lower than 29 μg/kg, e.g., 10 μg/kg or 1 μg/kg. In other embodiments, a priming dose is equal to the first treatment dose. A priming dose can be administered in a single administration or, alternatively, administered over multiple administrations (e.g., two). In some embodiments, one third of a priming dose is administered on a first day, and two thirds of the priming dose is administered on a second day, for example the day after the first day.

In an embodiment, the priming dose ranges from about 0.5 μg/kg to about 6 μg/kg or about 25 μg to about 480 μg. In another embodiment, the priming dose is about 1 μg/kg or about 50 μg to about 80 μg. In another embodiment, the priming dose is about 2 μg/kg or about 100 μg to about 160 μg. In another embodiment, the priming dose is about 3 μg/kg or about 150 μg to about 240 μg. In another embodiment, the priming dose is about 4 μg/kg or about 200 μg to about 320 μg. In another embodiment, the priming dose is about 5 μg/kg or about 250 μg to about 400 μg. In another embodiment, the priming dose is about 6 μg/kg or about 300 μg to about 480 μg.

### 7.2. Dosing Time

The dosing can be done over a number hours. For example, if the dosing of the BCMA binding molecule is done intravenously, it can be done via infusion. The infusion can occur over a span over about 30 minutes to about 6 hours. In some embodiments, the infusion can occur over a span of about 30 minutes. In some embodiments, the infusion can occur over a span of about 1 hour. In some embodiments, the infusion can occur over a span of about 1.5 hours. In some embodiments, the infusion can occur over a span of about 2 hours. In some embodiments, the infusion can occur over a span of about 2.5 hours. In some embodiments, the infusion can occur over a span of about 3 hours. In some embodiments, the infusion can occur over a span of about 3.5 hours. In some embodiments, the infusion can occur over a span of about 4 hours. In some embodiments, the infusion can occur over a span of about 4.5 hours. In some embodiments, the infusion can occur over a span of about 5 hours. In some embodiments, the infusion can occur over a span of about 5.5 hours. In some embodiments, the infusion can occur over a span of about 6 hours.

In some embodiments, the infusion can occur over a span of about 30 minutes to about 1 hour. In some embodiments, the infusion can occur over a span of about 1 hour to about 2 hours. In some embodiments, the infusion can occur over a span of about 2 hours to about 3 hours. In some embodiments, the infusion can occur over a span of about 3 hours to about 4 hours. In some embodiments, the infusion can occur over a span of about 4 hours to about 5 hours. In some embodiments, the infusion can occur over a span of about 5 hours to about 6 hours.

In some embodiments, the infusion can occur over a span of about 30 minutes to about 6 hours. In some embodiments, the infusion can occur over a span of about 1 hour to about 5 hours. In some embodiments, the infusion can occur over a span of about 1.5 hours to about 4 hours. In some embodiments, the infusion can occur over a span of about 2 hours to about 3 hours.

Further, any of the dosing time disclosed throughout can be used to dose the BCMA binding molecule and/or any of the other therapeutic agents disclosed throughout.

### 7.3. Dosing Schedule

In some embodiments, the BCMA binding molecule can be dosed once a week. In some embodiments, the BCMA binding molecule can be dosed twice a week. In some embodiments, the BCMA binding molecule can be dosed once every two weeks.

In some embodiments, the BCMA binding molecule can be dosed a single time. In some embodiments, the BCMA molecule can be dosed twice. In some embodiments, the BCMA binding molecule can be dosed three times. In some embodiments, the BCMA binding molecule can be dosed four times.

In some embodiments, the BCMA binding molecule can be dosed for 1 week. In some embodiments, the BCMA binding molecule can be dosed for 2 weeks. In some embodiments, the BCMA binding molecule can be dosed for 3 weeks. In some embodiments, the BCMA binding molecule can be dosed for 4 weeks.

In some embodiments, the BCMA binding molecule can be dosed until remission (with regards to cancers), e.g., until a response is observed. In some embodiments, the BCMA binding molecule can be dosed until partial remission, e.g., until a partial response is observed. In some embodiments, the BCMA binding molecule can be dosed until complete remission, e.g., until a complete response is observed.

With regards to any priming doses given, the priming dose can be given prior to a first treatment dose at any time before the treatment dose is given. For example, the priming dose can be given once a week before the first treatment dose is given. In another example, the priming dose can be given twice within one week before the first treatment dose is given.

In some embodiments, one third of a priming dose is administered to the subject on day 1 of a course of treatment, with the remainder of the priming dose administered on day 2 of the treatment. In some embodiments, a first treatment dose is subsequently administered to the subject one of days 5-11 of the treatment (e.g., one of days 6-10, one of days 7-9 or day 8), a second treatment dose is subsequently administered to the subject one of days 12-18 of the treatment (e.g., one of days 13-17, one of days 14-16, or day 15), and a third treatment dose is subsequently administered to the subject one of days 19-25 (e.g., one of days 20-24, one of days 21-23, or day 22) of the treatment.

### 7.4. Side Effect Reducing Agents

With regards to side effect reducing agents, the agents and doses as described throughout the disclosure can be used in the manner as described throughout the disclosure. Further, these side effect reducing agents can be used as known to be safe and effective.

### 8. COMBINATIONS

A BCMA binding molecule of the disclosure can be used in combination other known agents and therapies. For example, the BCMA binding molecules can be used in treatment regimens in combination with surgery, chemotherapy, antibodies, radiation, peptide vaccines, steroids, cytoxins, proteasome inhibitors, immunomodulatory drugs (e.g., IMiDs), BH3 mimetics, cytokine therapies, stem cell transplant or any combination thereof.

For convenience, an agent that is used in combination with a BCMA binding molecule is referred to herein as an “additional” agent.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. The term “concurrently” is not limited to the administration of therapies (e.g., a BCMA binding molecule and an additional agent) at exactly the same time, but rather it is meant that a pharmaceutical composition comprising a BCMA binding molecule is administered to a subject in a sequence and within a time interval such that the BCMA binding molecules can act together with the additional therapy(ies) to provide an increased benefit than if they were administered otherwise. For example, each therapy can be administered to a subject at the same time or sequentially in any order at different points in time; however, if not administered at the same time, they should be administered sufficiently close in time so as to provide the desired therapeutic effect.

A BCMA binding molecule and one or more additional agents can be administered simultaneously, in the same or in separate compositions, or sequentially. For sequential administration, the BCMA binding molecule can be administered first, and the additional agent can be administered second, or the order of administration can be reversed.

The BCMA binding molecule and the additional agent(s) can be administered to a subject in any appropriate form and by any suitable route. In some embodiments, the routes of administration are the same. In other embodiments the routes of administration are different.

In other embodiments, the delivery of one treatment ends before the delivery of the other treatment begins.

In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The BCMA binding molecules and/or additional agents can be administered during periods of active disorder, or during a period of remission or less active disease. A BCMA binding molecule can be administered before the treatment with the additional agent(s), concurrently with the treatment with the additional agent(s), post-treatment with the additional agent(s), or during remission of the disorder.

When administered in combination, the BCMA binding molecule and/or the additional agent(s) can be administered in an amount or dose that is higher, lower or the same than the amount or dosage of each agent used individually, e.g., as a monotherapy.

The additional agent(s) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising a BCMA binding molecule is administered to a subject in a sequence and within a time interval such that the molecules of the disclosure can act together with the additional therapy(ies) to provide an increased benefit than if they were administered otherwise. For example, each therapy can be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route.

The BCMA binding molecule and the additional agent(s) can be administered to a subject by the same or different routes of administration.

The BCMA binding molecules and the additional agent(s) can be cyclically administered. Cycling therapy involves the administration of a first therapy (e.g., a first prophylactic or therapeutic agent) fora period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain instances, the one or more additional agents, are other anti-cancer agents, anti-allergic agents, anti-nausea agents (or anti-emetics), pain relievers, cytoprotective agents, and combinations thereof.

The BCMA binding molecule can be used in combination with a gamma secretase inhibitor (“GSI”).

Accordingly, in one aspect, the disclosure provides a method for treating subjects that have a disease associated with expression of BCMA, comprising administering to the subject an effective amount of: (i) a BCMA binding molecule, and (ii) a gamma secretase inhibitor (GSI).

In another aspect aspect, the disclosure provides a method for treating subjects that have undergone treatment for a disease associated with expression of BCMA, comprising administering to the subject an effective amount of: (i) a BCMA binding molecule, and (ii) a GSI.

In one embodiment, the BCMA binding molecule and the GSI are administered simultaneously or sequentially. In one embodiment, the BCMA binding molecule is administered prior to the administration of the GSI. In one embodiment, the GSI is administered prior to the administration of the BCMA binding molecule. In one embodiment, the BCMA binding molecule and the GSI are administered simultaneously.

In one embodiment, the GSI is administered prior to the administration of the BCMA binding molecule (e.g., GSI is administered 1, 2, 3, 4, or 5 days prior to the administration of the BCMA binding molecule), optionally where after the administration of the GSI and prior to the administration of the BCMA binding molecule, the subject shows an increase in cell surface BCMA expression levels and/or a decrease in soluble BCMA levels.

In some embodiments, the GSI is a small molecule that reduces the expression and/or function of gamma secretase, e.g., a small-molecule GSI disclosed herein. In one embodiment, the GSI is chosen from LY-450139, PF-5212362, BMS-708163, MK-0752, ELN-318463, BMS-299897, LY-411575, DAPT, AL-101 (also known as BMS-906024), AL-102 (also known as BMS-986115), PF-3084014, R04929097, and LY3039478. In one embodiment, the GSI is chosen from PF-5212362, ELN-318463, BMS-906024, and LY3039478. Exemplary GSIs are disclosed in Takebe et al., Pharmacol Ther. 2014 Feb;141(2):140-9; and Ran et al., EMBO Mol Med. 2017 July; 9(7):950-966. In some embodiments, the GSI is AL-101. In some embodiments, the GSI is AL-102. The structure of AL-102 is shown below:

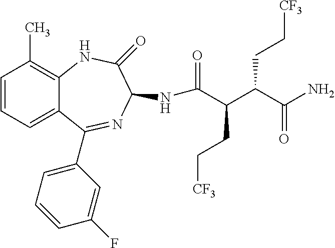 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

In some embodiments, MK-0752 is administered in combination with docetaxel. In some embodiments, MK-0752 is administered in combination with gemcitabine. In some embodiments, BMS-906024 is administered in combination with chemotherapy.

In a further embodiment, the GSI is a compound described in U.S. Pat. No. 7,468,365. In one embodiment, the GSI is LY-450139, i.e., semagacestat, (S)-2-hydroxy-3-methyl-N—((S)-1-(((S)-3-methyl-2-oxo-2,3,4,5-tetrahydro-1H-benzo[d]azepin-1-yl)amino)-1-oxopropan-2-yl)butanamide, or a pharmaceutically acceptable salt thereof. In one embodiment, the GSI is

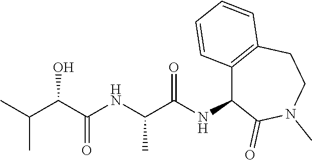 [SMILES: CC(C)[C@H](O)C(=O)N[C@@H](C)C(=O)N[C@@H]1C(=O)N(C)CCc2ccccc21]

or a pharmaceutically acceptable salt thereof.

In a further embodiment, the GSI is a compound described in U.S. Pat. No. 7,687,666. In one embodiment, the GSI is PF-5212362, i.e., begacestat, GSI-953, or (R)-5-chloro-N-(4,4,4-trifluoro-1-hydroxy-3-(trifluoromethyl)butan-2-yl)thiophene-2-sulfonamide, a pharmaceutically acceptable salt thereof. In one embodiment, the GSI is

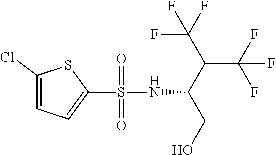 [SMILES: O=S(=O)(N[C@@H](CO)C(C(F)(F)F)C(F)(F)F)c1ccc(Cl)s1]

or a pharmaceutically acceptable salt thereof.

In a further embodiment, the GSI is a compound described in U.S. Pat. No. 8,084,477. In one embodiment, the GSI is BMS-708163, i.e., avagacestat, or (R)-2-((4-chloro-N-(2-fluoro-4-(1,2,4-oxadiazol-3-yl)benzyl)phenyl)sulfonamido)-5,5,5-trifluoropentanamide, or a pharmaceutically acceptable salt thereof. In one embodiment, the GSI is

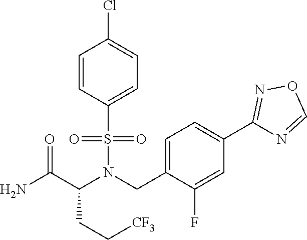 [SMILES: NC(=O)[C@@H](CCC(F)(F)F)N(Cc1ccc(-c2ncon2)cc1F)S(=O)(=O)c1ccc(Cl)cc1]

or a pharmaceutically acceptable salt thereof.

In some embodiments, the GSI is a compound described in U.S. Pat. No. 7,160,875. In one embodiment, the GSI is RO4929097, i.e., (S)-2,2-dimethyl-N1-(6-oxo-6,7-dihydro-5H-dibenzo[b,d]azepin-7-yl)-N3-(2,2,3,3,3-pentafluoropropyl)malonamide, or a pharmaceutically acceptable salt thereof. In one embodiment, the GSI is

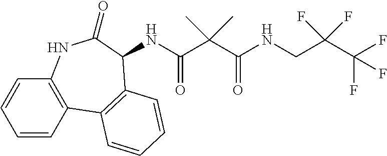 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In some embodiments, the GSI is

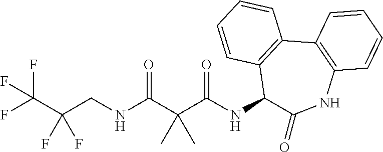 [SMILES: CC(C)(C(=O)NCC(F)(F)C(F)(F)F)C(=O)N[C@@H]1C(=O)Nc2ccccc2-c2ccccc21]

or a pharmaceutically acceptable salt thereof.

In some embodiments, the GSI is a compound described in U.S. Pat. No. 6,984,663. In one embodiment, the GSI is MK-0752, i.e., 3-((1S,4R)-4-((4-chlorophenyl)sulfonyl)-4-(2,5-difluorophenyl)cyclohexyl)propanoic acid, or a pharmaceutically acceptable salt thereof. In some embodiments, the GSI is

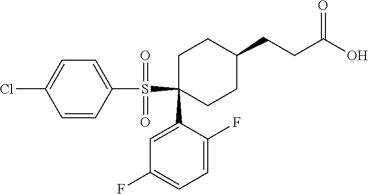 [SMILES: O=C(O)CC[C@H]1CC[C@@](c2cc(F)ccc2F)(S(=O)(=O)c2ccc(Cl)cc2)CC1]

or a pharmaceutically acceptable salt thereof.

In some embodiments, the GSI is a compound described in U.S. Pat. No. 7,795,447. In one embodiment, the GSI is PF-3084014, i.e., nirogacestat or (S)-2-(((S)-6,8-difluoro-1,2,3,4-tetrahydronaphthalen-2-yl)amino)-N-(1-(2-methyl-1-(neopentylamino)propan-2-yl)-1H-imidazol-4-yl)pentanamide, or a pharmaceutically acceptable salt thereof.

In some embodiments, the GSI is

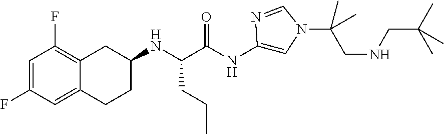 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof.

In some embodiments, the GSI is a compound described in U.S. Pat. No. 7,939,657. In one embodiment, the GSI is ELN-318463, i.e., HY-50882 or (R)—N-(4-bromobenzyI)-4-chloro-N-(2-oxoazepan-3-yl)benzenesulfonamide, or a pharmaceutically acceptable salt thereof. In some embodiments, the GSI is

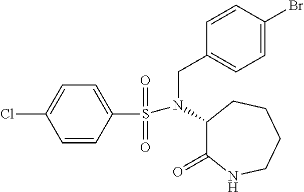 [SMILES: O=C1NCCCC[C@H]1N(Cc1ccc(Br)cc1)S(=O)(=O)c1ccc(Cl)cc1]

or a pharmaceutically acceptable salt thereof.

In some embodiments, the GSI is a compound described in U.S. Pat. No. 8,629,136. In one embodiment, the GSI is BMS-906024, i.e., (2R,3S)—N-[(3S)-1-methyl-2-oxo-5-phenyl-2,3-dihydro-1H-1,4-benzodiazepin-3-yl]-2,3-bis(3,3,3-trifluoropropyl)succinamide, or a pharmaceutically acceptable salt thereof. In one embodiment, the GSI is

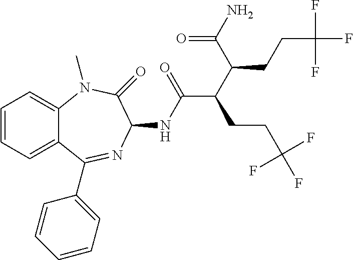 [SMILES: CN1C(=O)[C@@H](NC(=O)[C@H](CCC(F)(F)F)[C@H](CCC(F)(F)F)C(N)=O)N=C(c2ccccc2)c2ccccc21]

or a pharmaceutically acceptable salt thereof.

In some embodiments, the GSI is a compound described in U.S. Pat. No. 8,629,136. In one embodiment, the GSI is LY3039478, i.e., crenigacestat or 4,4,4-trifluoro-N—((R)-1-(((S)-5-(2-hydroxyethyl)-6-oxo-6,7-dihydro-5H-benzo[d]pyrido[2,3-b]azepin-7-yl)amino)-1-oxopropan-2-yl)butanamide, or a pharmaceutically acceptable salt thereof. In some embodiments, the GSI is:

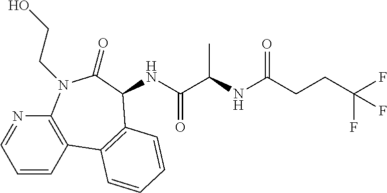 [SMILES: C[C@@H](NC(=O)CCC(F)(F)F)C(=O)N[C@@H]1C(=O)N(CCO)c2ncccc2-c2ccccc21]

or a pharmaceutically acceptable salt thereof.

In some embodiments, the GSI is BMS-299897, i.e., 2-[(1R)-1-[[(4-chlorophenyl)sulfonyl](2,5-difluorophenyl)amino]ethyl-5-fluorobenzenebutanoic acid or a pharmaceutically acceptable salt thereof. In some embodiments, the GSI is

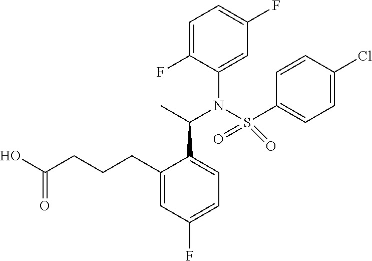 [SMILES: C[C@H](c1ccc(F)cc1CCCC(=O)O)N(c1cc(F)ccc1F)S(=O)(=O)c1ccc(Cl)cc1]

or a pharmaceutically acceptable salt thereof.

In some embodiments, the GSI is LY-411575, i.e., LSN-411575, (S)-2-((S)-2-(3,5-difluorophenyl)-2-hydroxyacetamido)-N—((S)-5-methyl-6-oxo-6,7-dihydro-5H-dibenzo[b,d]azepin-7-yl)propanamide, or a pharmaceutically acceptable salt thereof. In some embodiments, the GSI is

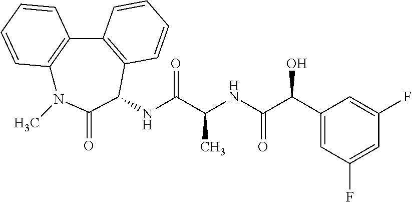 [SMILES: C[C@H](NC(=O)[C@@H](O)c1cc(F)cc(F)c1)C(=O)N[C@@H]1C(=O)N(C)c2ccccc2-c2ccccc21]

or a pharmaceutically acceptable salt thereof.

In some embodiments, the GSI is DAPT, i.e., N-[(3,5-difluorophenyl)acetyl]-L-alanyl-2-phenyl]glycine-1,1-dimethylethyl ester or a pharmaceutically acceptable salt thereof. In some embodiments, the GSI is

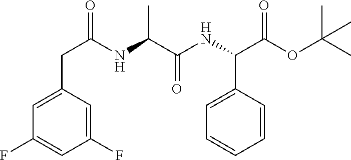 [SMILES: C[C@H](NC(=O)Cc1cc(F)cc(F)c1)C(=O)N[C@H](C(=O)OC(C)(C)C)c1ccccc1]

or a pharmaceutically acceptable salt thereof.

In some embodiments, the GSI is a compound described in U.S. Patent Publication No. US-2015-307533 (e.g., in the Table on pages 13-16). In some embodiments, the GSI is selected from:

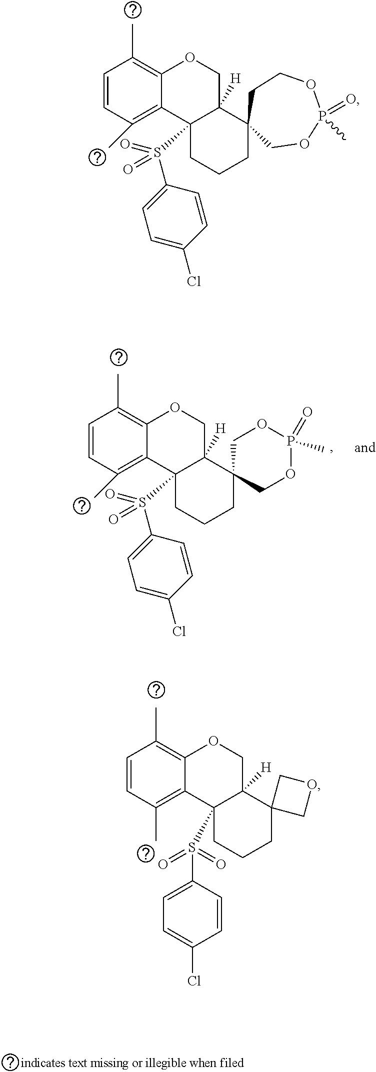

or a pharmaceutically acceptable salt thereof.

In some embodiments, the GSI is a compound in U.S. Pat. No. 8,188,069. In one embodiment, the GSI is

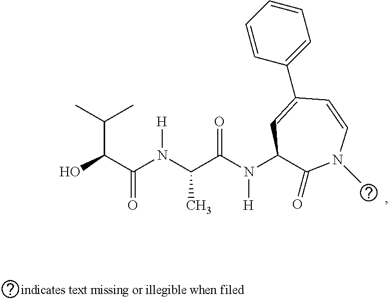 [SMILES: CC(C)[C@H](O)C(=O)N[C@@H](C)C(=O)N[C@H]1C=C(c2ccccc2)C=CN(C)C1=O]

or a pharmaceutically acceptable salt thereof.

In some embodiments, the GSI is a compound described in U.S. Pat. No. 9,096,582 (e.g., in the Table on pages 13-17). In some embodiments, the GSI is selected from:

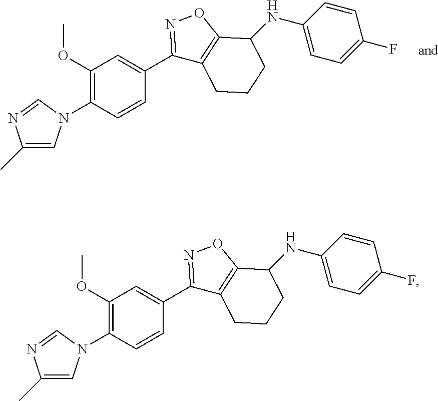 [SMILES: COc1cc(-c2noc3c2CCCC3Nc2ccc(F)cc2)ccc1-n1cnc(C)c1 ; COc1cc(-c2noc3c2CCCC3Nc2ccc(F)cc2)ccc1-n1cnc(C)c1]

or a pharmaceutically acceptable salt thereof.

In some embodiments, the GSI is a compound described in U.S. Patent Publication No. US-2011-0257163 (e.g., in paragraphs [0506] to [0553]) In some embodiments, the GSI is selected from:

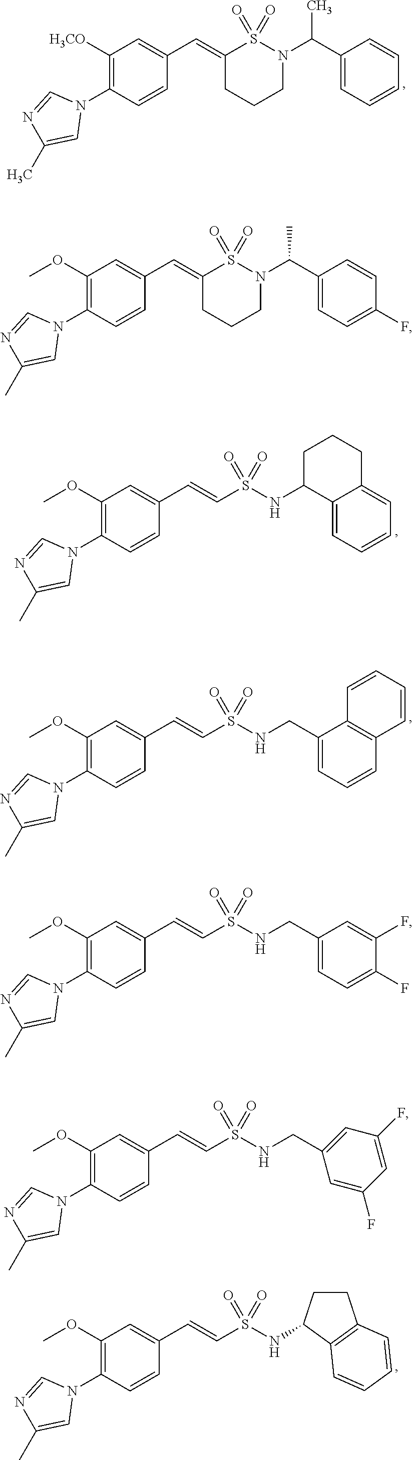

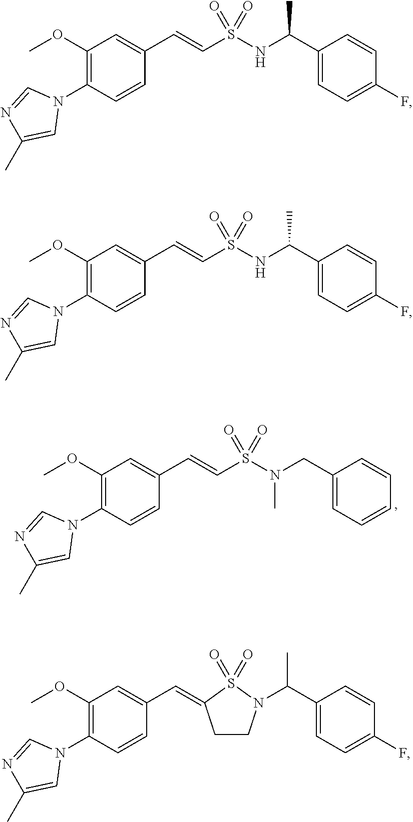 [SMILES: COc1cc(/C=C/S(=O)(=O)N[C@@H](C)c2ccc(F)cc2)ccc1-n1cnc(C)c1 ; COc1cc(/C=C/S(=O)(=O)N[C@H](C)c2ccc(F)cc2)ccc1-n1cnc(C)c1 ; COc1cc(/C=C/S(=O)(=O)N(C)Cc2ccccc2)ccc1-n1cnc(C)c1 ; COc1cc(/C=C2\CCN(C(C)c3ccc(F)cc3)S2(=O)=O)ccc1-n1cnc(C)c1]

and pharmaceutically acceptable salts thereof.

In some embodiments, the GSI is selected from:

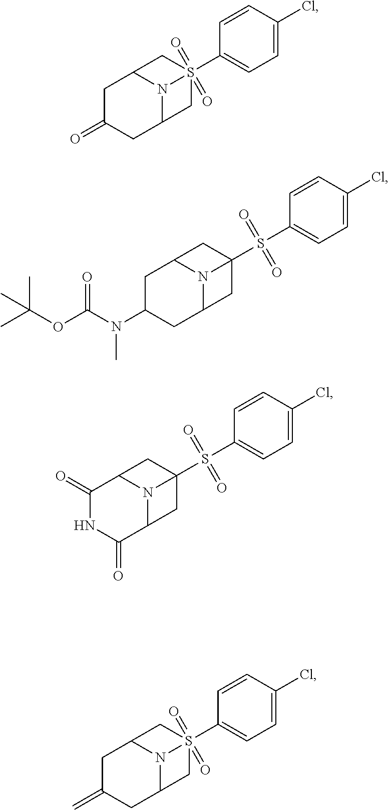

and pharmaceutically acceptable salts thereof.

In some embodiments, the GSI is selected from:

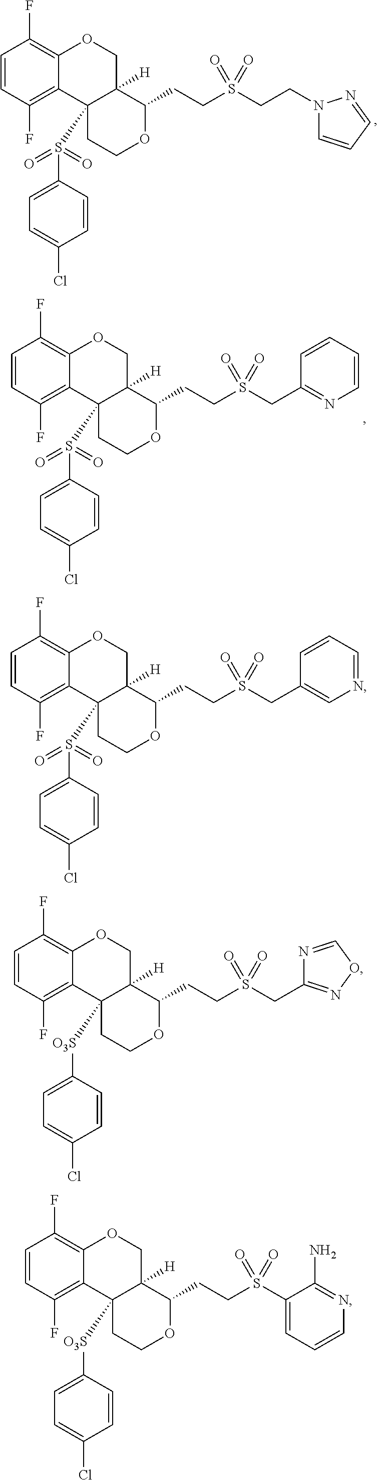

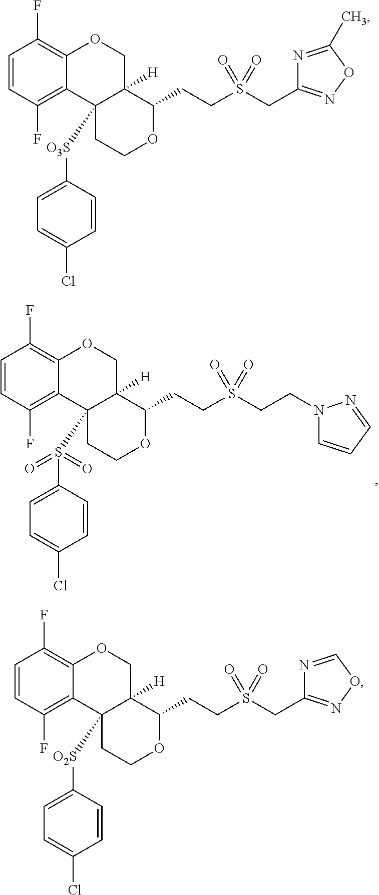

and pharmaceutically acceptable salts thereof.

In some embodiments, the GSI is an antibody molecule that reduces the expression and/or function of gamma secretase. In some embodiments, the GSI is an antibody molecule targeting a subunit of gamma secretase. In some embodiments, the GSI is chosen from an anti-presenilin antibody molecule, an anti-nicastrin antibody molecule, an anti-APH-1 antibody molecule, or an anti-PEN-2 antibody molecule.

Exemplary antibody molecules that target a subunit of gamma secretase (e.g., e.g., presenilin, nicastrin, APH-1, or PEN-2) are described in U.S. Pat. Nos. 8,394,376, 8,637,274, and 5,942,400.

In one aspect, the disclosure provides a method for treating subjects having a B cell condition or disorder, comprising administering to the subject an effective amount of: (i) a BCMA binding molecule, and (ii) a gamma secretase modulator (e.g., a GSI). Exemplary B cell conditions or disorders that can be treated with the combination of a BCMA binding molecule and a gamma secretase modulator include multiple myeloma, Waldenstrom's macroglobulinemia, chronic lymphocytic leukemia, B cell non-Hodgkin's lymphoma, plasmacytoma, Hodgkins' lymphoma, follicular lymphomas, small non-cleaved cell lymphomas, endemic Burkitt's lymphoma, sporadic Burkitt's lymphoma, marginal zone lymphoma, extranodal mucosa-associated lymphoid tissue lymphoma, nodal monocytoid B cell lymphoma, splenic lymphoma, mantle cell lymphoma, large cell lymphoma, diffuse mixed cell lymphoma, immunoblastic lymphoma, primary mediastinal B cell lymphoma, pulmonary B cell angiocentric lymphoma, small lymphocytic lymphoma, B cell proliferations of uncertain malignant potential, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, rapidly progressive glomerulonephritis, heavy-chain disease, primary or immunocyte-associated amyloidosis, and monoclonal gammopathy of undetermined significance.

In some embodiments, the gamma secretase modulator is a gamma secretase modulator described in WO 2017/019496. In some embodiments, the gamma secretase modulator is γ-secretase inhibitor I (GSI I) Z-Leu-Leu-Norleucine; γ-secretase inhibitor II (GSI II); γ-secretase inhibitor III (GSI III), N-Benzyloxycarbonyl-Leu-leucinal, N-(2-Naphthoyl)-Val-phenylalaninal; γ-secretase inhibitor IV (GSI IV); γ-secretase inhibitor V (GSI V), N-Benzyloxycarbonyl-Leu-phenylalaninal; γ-secretase inhibitor VI (GSI VI), 1-(S)-endo-N-(1,3,3)-Trimethylbicyclo[2.2.1]hept-2-yl)-4-fluorophenyl Sulfonamide; γ-secretase inhibitor VII (GSI VII), Menthyloxycarbonyl-LL-CHO; γ-secretase inhibitor IX (GSI IX), (DAPT), N—[N-(3,5-Difluorophenacetyl-L-alanyl)]-S-phenylglycine t-Butyl Ester; γ-secretase inhibitor X (GSI X), {1S-Benzyl-4R-[1-(1S-carbamoyl-2-phenethylcarbamoyl)-1S-3-methylbutylcarbamoyl]-2R-hydroxy-5-phenylpentyl}carbamic Acid tert-butyl Ester; γ-secretase inhibitor XI (GSI XI), 7-Amino-4-chloro-3-methoxyisocoumarin; γ-secretase inhibitor XII (GSI XII), Z-Ile-Leu-CHO; γ-secretase inhibitor XIII (GSI XIII), Z-Tyr-Ile-Leu-CHO; γ-secretase inhibitor XIV (GSI XIV), Z-Cys(t-Bu)-Ile-Leu-CHO; γ-secretase inhibitor XVI (GSI XVI), N—[N-3,5-Difluorophenacetyl]-L-alanyl-S-phenylglycine Methyl Ester; γ-secretase inhibitor XVII (GSI XVII); γ-secretase inhibitor XIX (GSI XIX), benzo[e][1,4]diazepin-3-yl)-butyramide; γ-secretase inhibitor XX (GSI XX), (S,S)-2-[2-(3,5-Difluorophenyl)acetylamino]-N-(5-methyl-6-oxo-6,7-dihydro-5H-dibenzo[b,d]azepin-7-yl)propionamide; γ-secretase inhibitor XXI (GSI XXI), (S,S)-2-[2-(3,5-Difluorophenyl)-acetylamino]-N-(I-methyl-2-oxo-5-phenyl-2-,3-dihydro-1H-benzo[e][1,4]diazepin-3-yl)-propionamide; Gamma40 secretase inhibitor I, N-trans-3,5-Dimethoxycinnamoyl-Ile-leucinal; Gamma40 secretase inhibitor II, N-tert-Butyloxycarbonyl-Gly-Val-Valinal; Isovaleryl-V V-Sta-A-Sta-OCH₃; MK-0752 (Merck); MRK-003 (Merck); semagacestat/LY450139 (Eli Lilly); RO4929097; PF-03084014; BMS-708163; MPC-7869 (γ-secretase modifier), YO-01027 (Dibenzazepine); LY411575 (Eli Lilly and Co.); L-685458 (Sigma-Aldrich); BMS-289948 (4-chloro-N-(2,5-difluorophenyl)-N-((IR)-{4-fluoro-2-[3-(1H-imidazol-1-yl)propyl]phenyl}ethyl)benzenesulfonamide hydrochloride); or BMS-299897 (4-[2-((IR)-I-{[(4-chlorophenyl)sulfonyl]-2,5-difluoroanilino}ethyl)-5-fluorophenyljbutanoic acid) (Bristol Myers Squibb).

### 9. THERAPEUTIC INDICATIONS

The BCMA binding molecules of the disclosure can be used in the treatment of any disease associated with BCMA expression. In one aspect, the disclosure provides a method of treating cancer in a subject. The method comprises administering to the subject a BCMA binding molecule such that the cancer is treated in the subject. An example of a cancer that is treatable by the BCMA-targeting agent is a cancer associated with expression of BCMA, such as multiple myeloma (also known as MM) (See Claudio et al., 2002, Blood. 100(6):2175-86; and Novak et al., 2004, Blood. 103(2):689-94). Multiple myeloma, also known as plasma cell myeloma or Kahler's disease, is a cancer characterized by an accumulation of abnormal or malignant plasma B-cells in the bone marrow. Frequently, the cancer cells invade adjacent bone, destroying skeletal structures and resulting in bone pain and fractures. Most cases of myeloma also feature the production of a paraprotein (also known as M proteins or myeloma proteins), which is an abnormal immunoglobulin produced in excess by the clonal proliferation of the malignant plasma cells. Blood serum paraprotein levels of more than 30 g/L is diagnostic of multiple myeloma, according to the diagnostic criteria of the International Myeloma Working Group (IMWG) (See Kyle et al., 2009, Leukemia. 23:3-9). Other symptoms or signs of multiple myeloma include reduced kidney function or renal failure, bone lesions, anemia, hypercalcemia, and neurological symptoms.

### 10. PATIENT POPULATION

The BCMA binding molecules can be used to treat subjects in need thereof. The subjects can be diagnosed with cancer, e.g., a blood cancer such as multiple myeloma. In some embodiments, the subjects can have previously been treated with one or more therapeutic agents. In some embodiments, the treatment may have failed.

In some embodiments, the subject has previously received one or more prior treatments for their disease. In some embodiments, the subject has previously received one prior treatment for their disease. In some embodiments, the subject has previously received one prior treatment for their disease. In some embodiments, the subject has previously received one prior treatment for their disease. In some embodiments, the subject has previously received one prior treatment for their disease.

In some embodiments, the subject previously received an IMiD, a proteasome inhibitor, an anti-CD38 antibody, or any combination thereof. In some embodiments, the subject previously received an IMiD. In some embodiments, the subject previously received a proteasome inhibitor. In some embodiments, the subject previously received an anti-CD38 antibody.

### 10.1. Inclusion Criteria

The subject that can be treated with the BCMA binding molecules can include a subject that has signed an informed consent form prior to being treated with the BCMA binding molecule.

The subject that can be treated with the BCMA binding molecules can include a subject that is a male or female subject that is greater than equal to 18 years of age.

The subject that can be treated with the BCMA binding molecules can include a subject that has an Estern Cooperative Oncology Group (ECOG) performance status of less than equal to two (2). The ECOG performance status can be determined at any time prior to being treated with the BCMA binding molecule.

The subject that can be treated with the BCMA binding molecules can include a subject that has a confirmed diagnosis of cancer. For example, the subject that can be treated with the BCMA binding molecule can be a subject that has a confirmed diagnosis of multiple myeloma. The subject can also have received two or more standard of care (SoC) regimens. The SoC regimens can include an IMiD (e.g. lenalidomide or pomalidomide), a proteasome inhibitor (e.g. bortezomib, carfilzomib), and/or an anti-CD38 agent (e.g. daratumumab. The subject can also be relapsed and/or refractory to, or intolerant of each regimen. The subject can also have documented evidence of disease progression (IMWG criteria) even after receiving previous treatments. The subject can have also previously received a prior autologous bone marrow transplant, a BCMA CAR-T or BCMA-ADC.

The subject that can be treated with the BCMA binding molecules can include a subject that has a measureable diseased defined by serum M-protein level of greater than equal to 1.0 g/dL. The subject that can be treated with the BCMA binding molecules can include a subject that has a measureable diseased defined by urine M-protein level of greater than equal to 200 mg/24 hours. The subject that can be treated with the BCMA binding molecules can include a subject that has a measureable diseased defined by serum free light chain (sFLC) of greater than 100 mg/L of involved FLC.

The subject that can be treated with the BCMA binding molecules can include a subject that is willing to undergo a serial bone marrow aspirate and/or biopsy. The serial bone marrow aspirate and/or biopsy can occur at any time prior to treatment with the BCMA binding molecule. The serial bone marrow aspirate and/or biopsy can occur at any time following treatment with the BCMA binding molecule. The serial bone marrow aspirate and/or biopsy can be performed for the assessment of disease status and biomarker/pharmacodynamics.

### 10.2. Exclusion Criteria

In some embodiments, the subject that can be treated with the BCMA binding molecules may not have or have had one or more of the following exclusion criteria disclosed in this Section 10.2. For example, in some embodiments if the subject has or has had any one of the following exclusion criteria disclosed in this Section 10.2, then they should not be treated with the BCMA binding molecule. As another example, the subject that can be treated with the BCMA binding molecules may not have or have had two or more of the following exclusion criteria disclosed in this Section 10.2. As another example, the subject that can be treated with the BCMA binding molecules may not have or have had three or more of the following exclusion criteria disclosed in this Section 10.2. As another example, the subject that can be treated with the BCMA binding molecules may not have or have had four or more of the following exclusion criteria disclosed in this Section 10.2. As another example, the subject that can be treated with the BCMA binding molecules may not have or have had five or more of the following exclusion criteria disclosed in this Section 10.2.

The subject that can be treated with the BCMA binding molecules can include a subject that may not have had previous radiotherapy. In other embodiments, the subject may have had previous radiotherapy. In some embodiments, the radiotherapy was not done within one month of the start of treatment. In some embodiments, the radiotherapy was not done within three weeks of the start of treatment. In some embodiments, the radiotherapy was not done within two weeks of the start of treatment. In some embodiments, the radiotherapy was not done within one week of the start of treatment.

Some exceptions for previous radiotherapy can be made, for example if the radiotherapy was localized. For example, the localized radiotherapy can have been for bone lesions, such as lytic bone lesions. Or in some cases, the localized radiotherapy can have been for phasmacytomas. Under these circumstances, the subject can be eligible for the treatment with the BCMA binding molecule.

The subject that can be treated with the BCMA binding molecules can include a subject that may not have had a recent major surgery. In some embodiments, the recent major surgery was not done within six months of the start of treatment. In some embodiments, the recent major surgery was not done within five months of the start of treatment. In some embodiments, the recent major surgery was not done within four months of the start of treatment. In some embodiments, the recent major surgery was not done within three months of the start of treatment. In some embodiments, the recent major surgery was not done within two months of the start of treatment. In some embodiments, the recent major surgery was not done within one month of the start of treatment. In some embodiments, the recent major surgery was not done within three weeks of the start of treatment. In some embodiments, the recent major surgery was not done within two weeks of the start of treatment. In some embodiments, the recent major surgery was not done within one week of the start of treatment.

The subject that can be treated with the BCMA binding molecules can include a subject that may not be using steroid therapy. In some embodiments, the steroid can be prednisone, dexamethasone, cortisol, equivalents thereof, or any other corticosteroids for human use. In some embodiments, the steroid therapy should not be chronic steroid therapy. For example, daily use of greater than equal to 10 mg of prednisone or equivalents can be considered chronic steroid therapy.

Some exceptions can be made if the steroids are topical, inhaled, nasal, or ophthalmic. Under these circumstances, the subject can be eligible for the treatment with the BCMA binding molecule.

The subject that can be treated with the BCMA binding molecule can include a subject that may not be using any immunosuppressive therapy/medication. In some embodiments, the immunosuppressive therapy/medication may not have been given within a month of treatment with the BCMA binding molecule. In some embodiments, the immunosuppressive therapy/medication may not have been given within four weeks of treatment with the BCMA binding molecule. In some embodiments, the immunosuppressive therapy/medication may not have been given within three weeks of treatment with the BCMA binding molecule. In some embodiments, the immunosuppressive therapy/medication may not have been given within two weeks of treatment with the BCMA binding molecule. In some embodiments, the immunosuppressive therapy/medication may not have been given within one week of treatment with the BCMA binding molecule. In some embodiments, the immunosuppressive medication is not a systemic treatment.

With regards to steroid therapy and/or immunosuppressive therapy, these considerations are independent of the potential pre-treatment, co-treatment, or post-treatment with immune suppressors in order to prevent/ameliorate any side effects (such as CRS) that is associated with treatment with a BCMA binding molecule. In other words, a person who is pre-/co-/post- with an immunosuppressive therapy as a part of the treatment regimen that comprises a BCMA binding molecule can still be eligible for the treatment with the BCMA binding molecule.

The subject that can be treated with the BCMA binding molecule can include a subject that may not have used any BCMA×CD3 bispecific antibody therapies in the past.

The subject that can be treated with the BCMA binding molecule can include a subject that may not have or have had a history of hypersensitivity reaction to any ingredient that contains the BCMA binding molecule. For example, the subject may not have or have had hypersensitivity reactions to any excipients in the formulation. In some embodiments, the subject may not have or have had hypersensitivity reactions to other monoclonal antibodies. In some embodiments, the hypersensitivity reactions are severe hypersensitivity reactions.

The subject that can be treated with the BCMA binding molecule can include a subject that may not have experienced toxicity with any previously treated BCMA targeted agents.

The subject that can be treated with the BCMA binding molecule can include a subject that does not have any malignant disease except for the disease that is being treated with the BCMA binding molecule. In other words, the subject can include a subject that does not have two or more malignant diseases, one of which is not being treated by the BCMA binding molecule.

Some exceptions can be made if the malignancies were previously treated and a complete response/remission of the malignancy was observed. In other words, if the previous treatments for the malignancy were curative. In some embodiments, the malignancy has not recurred within the past five years. In some embodiments, the malignancy has not recurred within the past four years. In some embodiments, the malignancy has not recurred within the past three years. In some embodiments, the malignancy has not recurred within the past two years. In some embodiments, the malignancy has not recurred within the past year. In some embodiments, the malignancy has not recurred within the past six months. Other exceptions can include subjects who had completely resected basal cell and squamous cell skin cancers. Further exceptions can include completely resected carcinoma in situ of any type. Under these circumstances, the subject can receive treatment with the BCMA binding molecule.

The subject that can be treated with the BCMA binding molecule can include a subject that does not have active autoimmune disease. The subject that can be treated with the BCMA binding molecule can include a subject that is not known to have an autoimmune disease. The subject that can be treated with the BCMA binding molecule can include a subject that is not suspected to have an autoimmune disease.

Some exceptions for autoimmune diseases can be made for subjects have vitiligo, hypothyroidism, or psoriasis. If the subject has hypothyroidism, the subject can have residual hypothyroidism. In some embodiments, if the subject has residual hypothyroidism, the subject that can be treated with the BCMA binding molecule only requires hormone replacement. If the subject has psoriasis, the subject that can be treated with the BCMA binding molecule does not require systemic treatment. In some embodiments, if the subject has psoriasis, the condition is not expected to recur. Under these circumstances, the subject can receive treatment with the BCMA binding molecule.

The subject that can be treated with the BCMA binding molecule can include a subject that has not been treated with a prohibited medication. In some embodiments, the subject has not been treated with a prohibited medication that cannot be discontinued at least three months prior to the start of treatment. In some embodiments, the subject has not been treated with a prohibited medication that cannot be discontinued at least two months prior to the start of treatment. In some embodiments, the subject has not been treated with a prohibited medication that cannot be discontinued at least one month prior to the start of treatment. In some embodiments, the subject has not been treated with a prohibited medication that cannot be discontinued at least four weeks prior to the start of treatment. In some embodiments, the subject has not been treated with a prohibited medication that cannot be discontinued at least three weeks prior to the start of treatment. In some embodiments, the subject has not been treated with a prohibited medication that cannot be discontinued at least two weeks prior to the start of treatment. In some embodiments, the subject has not been treated with a prohibited medication that cannot be discontinued at least one week prior to the start of treatment. In some embodiments, the subject has not been treated with a prohibited medication that cannot be discontinued at least six days prior to the start of treatment. In some embodiments, the subject has not been treated with a prohibited medication that cannot be discontinued at least five days prior to the start of treatment. In some embodiments, the subject has not been treated with a prohibited medication that cannot be discontinued at least four days prior to the start of treatment. In some embodiments, the subject has not been treated with a prohibited medication that cannot be discontinued at least three days prior to the start of treatment. In some embodiments, the subject has not been treated with a prohibited medication that cannot be discontinued at least two days prior to the start of treatment. In some embodiments, the subject has not been treated with a prohibited medication that cannot be discontinued at least one day prior to the start of treatment.

The subject that can be treated with the BCMA binding molecule can include a subject that does not have greater than equal to grade 2 neuropathy.

The subject that can be treated with the BCMA binding molecule can include a subject that does not have greater than or equal to grade 1 residual toxic effects from any previous therapy.

The subject that can be treated with the BCMA binding molecule can include a subject that does not have plasma cell leukemia and other plasmacytoid disorders, other than multiple myeloma.

The subject that can be treated with the BCMA binding molecule can include a subject that does not have a clinical laboratory result of an absolute neutrophil count (ANC) of greater than 1,000/mm3 without growth factor support. This ANC count can be measured 1 month prior to the start of treatment. In some embodiments, the ANC count can be measured 4 weeks prior to the start of treatment. In some embodiments, the ANC count can be measured 3 weeks prior to the start of treatment. In some embodiments, the ANC count can be measured 2 weeks prior to the start of treatment. In some embodiments, the ANC count can be measured 1 week prior to the start of treatment. In some embodiments, the ANC count can be measured 6 days prior to the start of treatment. In some embodiments, the ANC count can be measured 5 days prior to the start of treatment. In some embodiments, the ANC count can be measured 4 days prior to the start of treatment. In some embodiments, the ANC count can be measured 3 days prior to the start of treatment. In some embodiments, the ANC count can be measured 2 days prior to the start of treatment. In some embodiments, the ANC count can be measured 1 day prior to the start of treatment.

The subject that can be treated with the BCMA binding molecule can include a subject that does not have a clinical laboratory result of a platelet count less than 75,000 mm3 without transfusion support. This platelet count can be measured 1 month prior to the start of treatment. In some embodiments, the platelet count can be measured 4 weeks prior to the start of treatment. In some embodiments, the platelet count can be measured 3 weeks prior to the start of treatment. In some embodiments, the platelet count can be measured 2 weeks prior to the start of treatment. In some embodiments, the platelet count can be measured 1 week prior to the start of treatment. In some embodiments, the platelet count can be measured 6 days prior to the start of treatment. In some embodiments, the platelet count can be measured 5 days prior to the start of treatment. In some embodiments, the platelet count can be measured 4 days prior to the start of treatment. In some embodiments, the platelet count can be measured 3 days prior to the start of treatment. In some embodiments, the platelet count can be measured 2 days prior to the start of treatment. In some embodiments, the platelet count can be measured 1 day prior to the start of treatment.

The subject that can be treated with the BCMA binding molecule can include a subject that does not have a clinical laboratory result of a bilirubin level that is greater than 1.5 times the upper limit of the normal range (ULN). In some embodiments, the bilirubin level can be greater than 1.1 times the ULN. In some embodiments, the bilirubin level can be greater than 1.2 times the ULN. In some embodiments, the bilirubin level can be greater than 1.3 times the ULN. In some embodiments, the bilirubin level can be greater than 1.4 times the ULN. In some embodiments, the bilirubin level can be greater than 1.6 times the ULN. In some embodiments, the bilirubin level can be greater than 1.7 times the ULN. In some embodiments, the bilirubin level can be greater than 1.8 times the ULN. In some embodiments, the bilirubin level can be greater than 1.9 times the ULN. In some embodiments, the bilirubin level can be greater than 2.0 times the ULN.

The subject that can be treated with the BCMA binding molecule can include a subject that does not have a clinical laboratory result of an aspartate aminotransferase (AST) level that is greater than 3 times the upper limit of the normal range (ULN). In some embodiments, the AST level can be greater than 1.5 times the ULN. In some embodiments, the AST level can be greater than 2.0 times the ULN. In some embodiments, the AST level can be greater than 2.5 times the ULN. In some embodiments, the AST level can be greater than 3.5 times the ULN. In some embodiments, the AST level can be greater than 4.0 times the ULN. In some embodiments, the AST level can be greater than 4.5 times the ULN. In some embodiments, the AST level can be greater than 5.0 times the ULN.

The subject that can be treated with the BCMA binding molecule can include a subject that does not have a clinical laboratory result of an alanine aminotransferase (ALT) level that is greater than 3 times the upper limit of the normal range (ULN). In some embodiments, the ALT level can be greater than 1.5 times the ULN. In some embodiments, the ALT level can be greater than 2.0 times the ULN. In some embodiments, the ALT level can be greater than 2.5 times the ULN. In some embodiments, the ALT level can be greater than 3.5 times the ULN. In some embodiments, the AST level can be greater than 4.0 times the ULN. In some embodiments, the ALT level can be greater than 4.5 times the ULN. In some embodiments, the ALT level can be greater than 5.0 times the ULN.

The subject that can be treated with the BCMA binding molecule can include a subject that does not have a clinical laboratory result of a calculated creatinine clearance less than 30 ml/min. In some embodiments, the calculated creatinine clearance less than 10 ml/min. In some embodiments, the calculated creatinine clearance less than 20 ml/min. In some embodiments, the calculated creatinine clearance less than 40 ml/min. In some embodiments, the calculated creatinine clearance less than 50 ml/min. The calculated creatinine clearance can be measured by any known method. For example, the Cockcroft-Gault equation can be used to calculate creatinine clearance.

The subject that can be treated with the BCMA binding molecule can include a subject that does not have impaired cardiac function. The subject that can be treated with the BCMA binding molecule does not have clinically significant cardiac disease. For example, the subject does not have clinically significant and/or uncontrolled heart disease such as congestive heart failure requiring treatment (e.g., NYHA Grade≥2), uncontrolled hypertension or clinically significant arrhythmia. In some embodiments, the subject does not have a QTcF>470 msec on screening ECG or congenital long QT syndrome. In some embodiments, the subject does not have acute myocardial infarction or unstable angina pectoris less than 3 months prior to treatment.

The subject that can be treated with the BCMA binding molecule can include a subject that does not have an active infection. In some embodiments, the subject does not have an active infection that requires systemic therapy. In some embodiments, the subject does not have any severe infection within one month before treatment. In some embodiments, the subject does not have any severe infection within four weeks before treatment. In some embodiments, the subject does not have any severe infection within three weeks before treatment. In some embodiments, the subject does not have any severe infection within two weeks before treatment. In some embodiments, the subject does not have any severe infection within one week before treatment.

The subject that can be treated with the BCMA binding molecule can include a subject that does not have POEMS syndrome (plasma cell dyscrasia with polyneuropathy, organomegaly, endocrinopathy, monoclonal protein, skin changes).

The subject that can be treated with the BCMA binding molecule can include a subject that does not have any prior allogeneic SCT.

The subject that can be treated with the BCMA binding molecule can include a subject that does not have human immunodeficiency virus (HIV infection).

The subject that can be treated with the BCMA binding molecule can include a subject that does not have active Hepatitis B (HBV) or Hepatitis C (HCV) infection. Some exceptions to the HBV/HCV requirement can be made if the disease is controlled under antiviral therapy. In some cases, the HBV/HCV is tested, for example, if the HBV or HCV is clinically indicated or if the patient has a history of HBV or HCV infection.

The subject that can be treated with the BCMA binding molecule can include a subject that will not use any live vaccines against infectious diseases during the treatment period. In some embodiments, the subject will not use any live vaccines within 2 weeks of treatment commencement. In some embodiments, the subject will not use any live vaccines within 3 weeks of treatment commencement. In some embodiments, the subject will not use any live vaccines within 4 weeks of treatment commencement. In some embodiments, the subject will not use any live vaccines within 1 month of treatment commencement. In some embodiments, the subject will not use any live vaccines within 2 months of treatment commencement. In some embodiments, the subject will not use any live vaccines within 3 months of treatment commencement.

The subject that can be treated with the BCMA binding molecule can include a subject that has not been treated with cytotoxic or small molecule targeted antineoplastics or any experimental therapy before treatment. In some embodiments, the subject has not been treated with the cytotoxic or small molecule targeted antineoplastics or any experimental therapy within 1 month prior to commencing treatment with the BCMA binding molecule. In some embodiments, the subject has not been treated with the cytotoxic or small molecule targeted antineoplastics or any experimental therapy within 4 weeks prior to commencing treatment with the BCMA binding molecule. In some embodiments, the subject has not been treated with the cytotoxic or small molecule targeted antineoplastics or any experimental therapy within 3 weeks prior to commencing treatment with the BCMA binding molecule. In some embodiments, the subject has not been treated with the cytotoxic or small molecule targeted antineoplastics or any experimental therapy within 2 weeks prior to commencing treatment with the BCMA binding molecule. In some embodiments, the subject has not been treated with the cytotoxic or small molecule targeted antineoplastics or any experimental therapy within 1 week prior to commencing treatment with the BCMA binding molecule. In some embodiments, the subject has not been treated with the cytotoxic or small molecule targeted antineoplastics or any experimental therapy within 10 half-lives prior to commencing treatment with the BCMA binding molecule. In some embodiments, the subject has not been treated with the cytotoxic or small molecule targeted antineoplastics or any experimental therapy within 7 half-lives prior to commencing treatment with the BCMA binding molecule. In some embodiments, the subject has not been treated with the cytotoxic or small molecule targeted antineoplastics or any experimental therapy within 5 half-lives prior to commencing treatment with the BCMA binding molecule. In some embodiments, the subject has not been treated with the cytotoxic or small molecule targeted antineoplastics or any experimental therapy within 4 half-lives prior to commencing treatment with the BCMA binding molecule. In some embodiments, the subject has not been treated with the cytotoxic or small molecule targeted antineoplastics or any experimental therapy within 3 half-lives prior to commencing treatment with the BCMA binding molecule. In some embodiments, the subject has not been treated with the cytotoxic or small molecule targeted antineoplastics or any experimental therapy within 2 half-lives prior to commencing treatment with the BCMA binding molecule.

The subject that can be treated with the BCMA binding molecule can include a subject that has not had the initiation of hematopoietic colony-stimulating growth factors (e.g. G-CSF, M-CSF), thrombopoietin mimetics or erythroid stimulating agents less than or equal to two weeks prior to start of treatment. In some cases, the initiation did not occur less than one month prior to the start of treatment. In some cases, the initiation did not occur less than four weeks prior to the start of treatment. In some cases, the initiation did not occur less than three weeks prior to the start of treatment. In some cases, the initiation did not occur less than one week prior to the start of treatment.

If the subject received thrombopoietin mimetics more than two weeks prior to the treatment of the BCMA binding molecule, and the subject is on a stable dose, they can receive the BCMA binding molecule.

The subject that can be treated with the BCMA binding molecules can include a subject that has not received GM-CSF.

The subject that can be treated with the BCMA binding molecule can include a subject that has not received intravenous IG infusions that were given for infection prophylaxis. In some embodiments, the IG infusions should have ended 3 months prior to the start of treatment with the BCMA binding molecule. In some embodiments, the IG infusions should have ended 2 months prior to the start of treatment with the BCMA binding molecule. In some embodiments, the IG infusions should have ended 1 month prior to the start of treatment with the BCMA binding molecule. In some embodiments, the IG infusions should have ended 4 weeks prior to the start of treatment with the BCMA binding molecule. In some embodiments, the IG infusions should have ended 3 weeks prior to the start of treatment with the BCMA binding molecule. In some embodiments, the IG infusions should have ended 4 weeks prior to the start of treatment with the BCMA binding molecule. In some embodiments, the IG infusions should have ended 1 week prior to the start of treatment with the BCMA binding molecule.

The subject that can be treated with the BCMA binding molecule can include a subject that does that have active central nervous system (CNS) involvement by malignancy or presence of symptomatic CNS metastases, or CNS metastases that require local CNS-directed therapy (such as radiotherapy or surgery), or increasing doses of corticosteroids within 2 weeks prior to the start of treatment. In some embodiments, the CNS issues should not have occurred 3 months prior to the start of treatment. In some embodiments, the CNS issues should not have occurred 2 months prior to the start of treatment. In some embodiments, the CNS issues should not have occurred 1 month prior to the start of treatment. In some embodiments, the CNS issues should not have occurred 4 weeks prior to the start of treatment. In some embodiments, the CNS issues should not have occurred 3 weeks prior to the start of treatment. In some embodiments, the CNS issues should not have occurred 1 week prior to the start of treatment.

The subject that can be treated with the BCMA binding molecule can include a subject that does have any serious medical or psychiatric illness likely to interfere with treatment with the BCMA binding molecule.

The subject that can be treated with the BCMA binding molecule can include a subject that is not pregnant or nursing (lactating). Pregnancy can be defined as the state of a female after conception and until the termination of gestation, confirmed by a positive hCG laboratory test.

The subject that can be treated with the BCMA binding molecule is, in some embodiments, not a woman of child-bearing potential, unless they are using effective methods of contraception (e.g., two) during dosing and for 6 months after the last dose of study drug, including one highly effective method. A woman of child-bearing potential can be defined as all women physiologically capable of becoming pregnant. Women can be considered post-menopausal and not of child bearing potential if they have had 12 months of natural (spontaneous) amenorrhea with an appropriate clinical profile (i.e. age appropriate, history of vasomotor symptoms) or have had surgical bilateral oophorectomy (with or without hysterectomy), total hysterectomy, or tubal ligation at least six weeks ago. In the case of oophorectomy alone, only when the reproductive status of the woman has been confirmed by follow up hormone level assessment is she considered not of child bearing potential.

Highly effective contraception methods include but are not limited to total abstinence, female sterilization, male sterilization, and use of oral, injected or implanted hormonal methods of contraception or placement of an intrauterine device (IUD) or intrauterine system (IUS), and other forms of hormonal contraception that have comparable efficacy (failure rate<1%) (e.g., hormone vaginal ring or transdermal hormone contraception). Other effective method of contraception include barrier methods of contraception such as condom or occlusive cap (diaphragm or cervical/vault caps) with spermicide. (e.g., foam, gel, film, cream, or vaginal suppository).

With regards to abstinence, periodic abstinence (e.g., calendar, ovulation, symptothermal, post-ovulation methods)) and withdrawal are not acceptable methods of contraception.

With regards to female sterilization, examples include but are not limited to surgical bilateral oophorectomy with or without hysterectomy), total hysterectomy, or tubal ligation at least six weeks before taking study treatment. In case of oophorectomy alone, only when the reproductive status of the woman has been confirmed by follow up hormone level assessment.

Regarding male sterilization, this must have occurred at least 6 months prior to screening. For female subjects, the vasectomized male partner should be the sole partner for that subject.

Regarding the use of oral contraception, in some embodiments, women must have been stable on the same pill for a minimum of three months before the commencement of treatment with the BCMA binding molecule.

### 11. EXAMPLES

### 11.1. Example 1

### Identification of BSBM3

BCMA is a cell surface receptor expressed on plasma cells, as well as other B-cell malignancies, particularly multiple myeloma. For effective pharmaceutical development, it is highly desirable to have an antibody that is cross-reactive with both human antigens as well as the corresponding antigen in a model non-human primate species, such as cynomolgus macaque, for the purpose of non-clinical pharmacokinetic and toxicology studies.

To identify antibodies that were cross-reactive with both human and cynomolgus BCMA, a naïve phage library containing human antibody fragments was subject to four rounds of panning against recombinant human and cynomolgus BCMA antigens. Approximately 400 c single phage colonies were picked from the fourth round panning and nine unique clones were chosen to be amplified and rescued as phage for phage ELISA. The clones were analyzed for their affinity to human and cyno BCMA.

One of the clones was subject to affinity maturation in the form of yeast surface scFvs. After multiple rounds of screening affinity matured anti-BCMA pools were cloned into a heterodimeric bispecific antibody format (FIG. 1), expressed in HEK 293 cells and tested for the ability to bind BCMA on tumor cells and the ability to activate T-cells in a target-dependent fashion using a Jurkat NFAT luciferase (JNL) reporter assay.

From these assays the bispecific binding molecule referred to herein as BSBM3 was identified. The sequences of BSBM3 are shown in Table 1 below:

TABLE 1
BSBM3 Sequences

| | SEQ ID | |
|---|---|---|
| Description | NO | Sequence |
| HC BCMA arm | 1 | QVQLVESGGGVVQPGRSLRLSCAASGFTVSSYG |
| | | MHWVRQAPGKGLEWVAVISYTGSNKYYADSVKG |
| | | RFTISRDNSKNTLYLQMNSLRAEDTAVYYCGGSG |
| | | YALHDDYYGLDVWGQGTLVTVSSASTKGPSVFPL |
| | | APSSKSTSGGTAALGCLVKDYFPEPVTVSWNSGA |
| | | LTSGVHTFPAVLQSSGLYSLSSVVTVPSSSLGTQT |
| | | YICNVNHKPSDTKVDKKVEPKSCDKTHTCPPCPA |
| | | PPVAGPSVFLFPPKPKDTLMISRTPEVTCVVVDVK |
| | | HEDPEVKFNWYVDGVEVHNAKTKPREEEYNSTY |
| | | RVVSVLTVLHQDWLNGKEYKCKVSNKALPAPIEK |
| | | TISKAKGQPREPQVYTLPPSREEMTKNQVSLTCD |
| | | VSGFYPSDIAVEWESDGQPENNYKTTPPVLDSDG |
| | | SFFLYSKLTVDKSRWEQGDVFSCSVMHEALHNH |
| | | YTQKSLSLSPGK |
| DNA HC | 4 | CAAGTGCAGCTCGTGGAGTCTGGAGGGGGAGT |
| | | CGTGCAGCCTGGACGCTCCCTGAGACTGTCCT |
| | | GTGCGGCTTCGGGATTCACTGTGTCCAGCTAC |
| | | GGCATGCATTGGGTCCGCCAAGCACCGGGAAA |
| | | AGGCCTGGAGTGGGTGGCCGTGATCTCCTACA |
| | | CCGGCTCAAACAAGTACTACGCCGACAGCGTG |
| | | AAGGGCCGGTTCACCATTTCAAGGGACAACTCC |
| | | AAGAATACCCTGTATCTGCAAATGAACTCGCTG |
| | | CGGGCAGAGGACACCGCCGTGTACTACTGCGG |
| | | TGGCTCCGGTTACGCCCTGCACGATGACTACTA |
| | | CGGGCTCGATGTCTGGGGACAGGGGACGCTCG |
| | | TGACTGTGTCCTCGGCTAGCACCAAGGGCCCG |
| | | TCAGTGTTTCCTCTGGCCCCAAGCTCCAAGTCC |
| | | ACCTCCGGTGGTACAGCCGCGTTGGGATGCTT |
| | | GGTCAAGGACTACTTTCCGGAACCCGTGACCGT |
| | | GTCCTGGAACTCCGGCGCCCTGACTAGCGGAG |
| | | TGCACACCTTCCCCGCTGTGCTGCAGTCTAGCG |
| | | GGCTGTATTCCCTCTCGTCCGTGGTCACCGTGC |
| | | CGTCCTCATCCCTGGGAACCCAGACCTACATTT |
| | | GCAACGTGAACCACAAGCCGTCAGACACCAAG |
| | | GTGGACAAGAAGGTGGAGCCGAAGTCCTGCGA |
| | | CAAGACCCATACTTGTCCTCCTTGCCCCGCTCC |
| | | ACCTGTGGCGGGACCTTCCGTGTTCCTTTTCCC |
| | | GCCGAAGCCGAAGGACACTCTGATGATCTCGC |
| | | GGACTCCCGAAGTCACTTGCGTGGTGGTGGAC |
| | | GTCAAACACGAAGATCCCGAGGTCAAGTTCAAT |
| | | TGGTACGTGGACGGGGTGGAAGTCCACAACGC |
| | | CAAGACTAAGCCGCGCGAGGAAGAGTACAATT |
| | | CCACTTACCGGGTCGTGTCGGTGCTGACTGTG |
| | | CTGCATCAGGACTGGCTGAACGGAAAGGAGTA |
| | | CAAGTGCAAAGTGTCGAACAAGGCCCTGCCTG |
| | | CACCAATCGAAAAGACCATTAGCAAAGCCAAGG |
| | | GCCAGCCGAGAGAACCCCAAGTCTACACTCTG |
| | | CCACCATCCCGCGAAGAAATGACCAAGAACCAA |
| | | GTGTCGCTGACGTGCGACGTGTCGGGATTCTA |
| | | CCCGTCCGATATTGCCGTGGAATGGGAGAGCG |
| | | ACGGCCAACCCGAGAACAACTACAAGACTACCC |
| | | CCCCCGTCTTGGATTCCGATGGTTCCTTCTTCC |
| | | TGTACTCCAAGCTGACCGTGGATAAGTCCCGAT |
| | | GGGAGCAGGGCGATGTGTTCTCGTGCTCCGTG |
| | | ATGCATGAAGCCCTGCACAACCACTATACCCAG |
| | | AAGTCACTGTCGCTGAGCCCTGGGAAG |
| LC BCMA arm | 2 | QSALTQPASVSGSPGQSITISCTGTSSDVGGYNY |
| | | VSWYQQHPGKAPKLMIYDVSNRLRGVSNRFSGS |
| | | KSGNTASLTISGLQAEDEADYYCSSYTSSSALYVF |
| | | GSGTKVTVLGQPKAAPSVTLFPPSSEELQANKAT |
| | | LVCLISDFYPGAVTVAWKADSSPVKAGVETTTPSK |
| | | QSNNKYAASSYLSLTPEQWKSHRSYSCQVTHEG |
| | | STVEKTVAPTECS |
| DNA LC | 5 | CAGTCGGCGCTGACTCAGCCCGCATCCGTGAG |
| | | CGGTTCACCGGGACAGAGCATCACCATTTCCTG |
| | | CACCGGAACCTCAAGCGACGTGGGCGGCTACA |
| | | ACTACGTGTCCTGGTATCAGCAGCACCCGGGA |
| | | AAGGCCCCAAAGCTCATGATCTACGACGTGTCC |
| | | AATAGACTGCGGGGAGTGTCCAACCGGTTCTC |
| | | GGGAAGCAAATCCGGCAACACTGCTTCCCTGA |
| | | CCATCAGCGGACTCCAGGCCGAAGATGAGGCC |
| | | GACTACTACTGCTCATCCTACACGTCCTCTTCG |
| | | GCGCTTTACGTGTTCGGGTCGGGGACCAAGGT |
| | | CACCGTCCTGGGCCAACCTAAGGCGGCGCCCT |
| | | CAGTGACCCTGTTCCCTCCGTCGTCTGAAGAAC |
| | | TCCAGGCCAACAAGGCCACCCTCGTGTGCCTG |
| | | ATTTCGGACTTCTACCCGGGAGCCGTCACTGTG |
| | | GCCTGGAAGGCCGACAGCAGCCCAGTGAAGGC |
| | | CGGCGTGGAAACTACCACCCCGTCCAAGCAGT |
| | | CCAACAATAAGTACGCAGCCAGCTCCTACCTGT |
| | | CCCTGACCCCCGAACAATGGAAGTCACACAGAT |
| | | CCTACTCCTGTCAAGTCACCCACGAGGGCAGC |
| | | ACTGTCGAAAAGACCGTGGCACCGACTGAGTG |
| | | CTCG |
| CD3 arm | 3 | EVQLVESGGGLVQPGGSLRLSCAASGFTFSTYA |
| | | MNWVRQAPGKGLEWVGRIRSKANNYATYYADSV |
| | | KGRFTISRDDSKNTLYLQMNSLRAEDTAVYYCVR |
| | | HGNFGDSYVSWFAYWGQGTLVTVSSGKPGSGK |
| | | PGSGKPGSGKPGSQAVVTQEPSLTVSPGGTVTL |
| | | TCGSSTGAVTTSNYANWVQQKPGKSPRGLIGGT |
| | | NKRAPGVPARFSGSLLGGKAALTISGAQPEDEAD |
| | | YYCALWYSNHWVFGGGTKLTVLEPKSSDKTHTC |
| | | PPCPAPPVAGPSVFLFPPKPKDTLMISRTPEVTCV |
| | | VVDVKHEDPEVKFNWYVDGVEVHNAKTKPREEQ |
| | | YNSTYRVVSVLTVLHQDWLNGKEYKCKVSNKALP |
| | | APIEKTISKAKGQPREPQVYTLPPSREQMTKNQV |
| | | KLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| DNA CD3 | 6 | GAAGTGCAGCTTGTGGAGTCCGGGGGAGGATT |
| | | GGTCCAACCCGGTGGCTCGCTGAGGCTGAGTT |
| | | GCGCCGCTTCGGGGTTTACCTTCAGCACCTAC |
| | | GCTATGAACTGGGTCAGACAGGCGCCTGGAAA |
| | | GGGTTTGGAGTGGGTCGGACGCATCCGGTCCA |
| | | AGGCCAACAACTACGCGACTTACTATGCCGACT |
| | | CCGTCAAGGGACGGTTCACCATCTCCCGGGAC |
| | | GACAGCAAGAACACCCTGTACCTCCAAATGAAC |
| | | TCCCTTCGGGCCGAAGATACCGCCGTGTACTAC |
| | | TGCGTGAGACACGGCAACTTCGGCGACTCCTA |
| | | CGTGTCCTGGTTTGCCTACTGGGGCCAGGGTA |
| | | CTCTCGTGACCGTGTCATCAGGAAAGCCAGGCT |
| | | CGGGGAAGCCTGGCTCCGGAAAGCCTGGGAG |
| | | CGGAAAGCCGGGATCGCAGGCTGTGGTCACCC |
| | | AGGAACCCTCCCTGACTGTGTCCCCGGGAGGA |
| | | ACCGTGACACTGACTTGTGGCAGCTCCACCGG |
| | | AGCCGTGACCACCTCCAACTACGCCAACTGGG |
| | | TGCAGCAAAAGCCAGGAAAGTCCCCTAGGGGG |
| | | CTGATCGGTGGCACGAACAAGCGGGCACCTGG |
| | | AGTGCCTGCCCGATTCTCGGGTAGCCTGCTGG |
| | | GGGGAAAAGCCGCCCTGACCATTTCGGGCGCT |
| | | CAGCCAGAGGACGAAGCCGACTATTACTGCGC |
| | | ACTCTGGTACTCCAACCACTGGGTGTTCGGTGG |
| | | AGGCACCAAGCTGACCGTGCTGGAGCCAAAGT |
| | | CAAGCGACAAAACTCACACTTGCCCTCCTTGTC |
| | | CGGCTCCTCCTGTGGCTGGTCCCTCCGTGTTC |
| | | CTCTTCCCGCCGAAGCCGAAGGACACCCTCAT |
| | | GATTTCCCGGACGCCCGAAGTCACTTGTGTGGT |
| | | GGTCGATGTGAAGCATGAGGACCCCGAAGTGA |
| | | AGTTCAATTGGTACGTGGATGGCGTGGAGGTC |
| | | CACAACGCCAAGACCAAGCCGCGCGAAGAACA |
| | | GTACAACAGCACCTACCGCGTCGTGAGCGTGC |
| | | TCACCGTGCTCCACCAAGATTGGCTGAACGGAA |
| | | AGGAGTACAAGTGCAAAGTGTCCAACAAGGCC |
| | | CTTCCTGCACCTATTGAAAAGACTATTAGCAAG |
| | | GCCAAGGGACAGCCCCGCGAACCTCAAGTGTA |
| | | CACTCTGCCGCCGTCCAGAGAGCAGATGACCA |
| | | AAAACCAGGTCAAGCTCACTTGTCTCGTGAAGG |
| | | GCTTCTACCCGTCCGATATCGCGGTCGAATGG |
| | | GAGTCAAACGGCCAGCCCGAGAACAACTACAA |
| | | GACTACCCCACCGGTGCTTGACTCCGACGGTT |
| | | CGTTCTTTCTGTACTCCAAGCTGACCGTGGACA |
| | | AGTCCCGGTGGCAGCAAGGGAATGTGTTCAGC |
| | | TGCTCCGTGATGCACGAAGCCCTGCATAACCAC |
| | | TACACCCAGAAGTCGCTCAGCCTGTCCCCTGGA |
| | | AAA |

The activity of BSBM3 was compared to that of ch2B4_C29, a BCMA-CD3 bispecific antibody in development for the treatment of multiple myeloma (see, WO2016/0166629). Preliminary data with bivalent BSBM3 and h2B4_C29 from KMS11 and PBMC/T cell co-culture studies indicate that bivalent BSBM3 mediates lower levels of cytokine induction than h2B4_C29 (data not shown), suggesting that patients treated with BSBM3 may have a reduced risk of cytokine release syndrome compared to patients treated with h2B4_C29. Preliminary data also indicates that T cells activated by h2B4_C29 in the presence of KMS11 cells mediate more TCR downregulation than T cells activated by bivalent BSBM3 (data not shown), suggesting that BSBM3 may exhibit more sustained anti-cancer activity than h2B4_C29. Further, in a KMS11 xenograft model, some preliminary data suggests that BSBM3 (as well as h2B4_C29) has greater anti-tumor activity compared to BCMA-CD3 bispecific molecules from EngMab and Janssen.

### 11.2. Example 2

### Characteristics of BSBM3

BSBM3 was produced in Chinese hamster ovary (CHO) cells and belongs to the IgG1 isotype subclass. As shown in FIG. 1, BSBM3 has a Fab domain targeting BCMA, a single-chain Fv (scFv) domain targeting CD3, and the Fc domain confers IgG-like in vivo persistence due to unmodified FcRn (neonatal Fc receptor) affinity. The Fc domain of BSBM3 contains substitutions that ablate binding to human Fcy receptors and reduce the risk of non-selective T cell activation via FcR (Fc receptor)-mediated crosslinking. Its affinity to BCMA and CD3 has been summarized in Table 2. Binding of multiple molecules of BSBM3 simultaneously with BCMA on multiple myeloma (MM) cells and the CD3 subunit of the T cell receptor (TCR) complex on T cells leads to TCR crosslinking and formation of a cytolytic immune synapse, resulting in activation of T cells and specific lysis of MM cells.

TABLE 2

| Antigen | Average KD (M) | Standard Deviation (M) |
|---|---|---|
| Human BCMA-Fc | 2.01E−10 | 2.40E−11 |
| Cyno BCMA-Fc | 1.29E−09 | 1.79E−10 |
| Mouse BCMA-Fc | 1.15E−09 | 8.00E−11 |
| Human CD3-biotin | 8.78E−09 | 3.83E−09 |

Data shown as mean and SD from three biological replicates.

### 11.3. Example 3

### Non-Clinical Pharmacology (In Vitro)

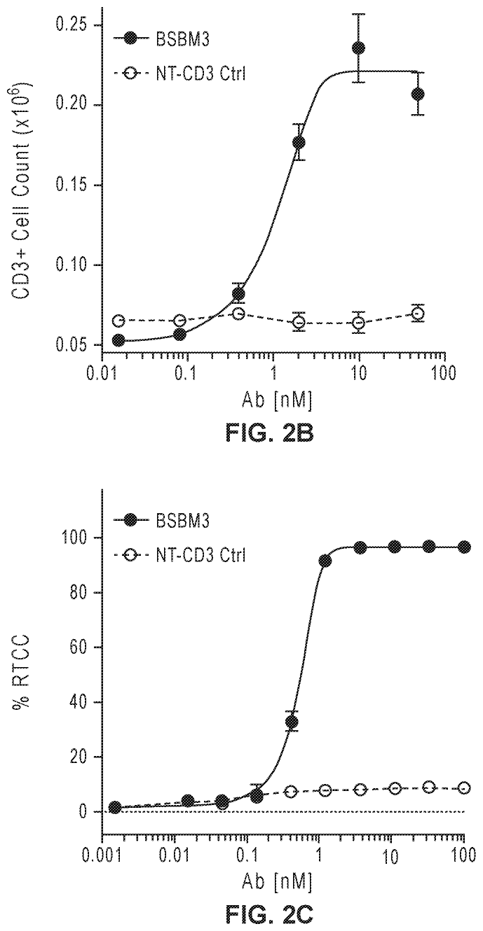

The activity of BSBM3 was characterized in an in vitro co-culture system with a BCMA+ myeloma cell line KMS11 and healthy donor T cells. BSBM3 induced T cell proliferation and cytokine secretion at concentrations≥1 nM (FIG. 2). Consistently, BSBM3 mediated potent redirected T cell cytotoxicity (RTCC) on KMS11 in a concentration-dependent manner (FIG. 2). In contrast, a non-targeting control antibody NT-CD3 (with the same anti-CD3 scFv but a non-targeting Fab instead of the anti-BCMA Fab) did not induce T cell proliferation or significant killing of KMS11 cells, indicating that specific binding to BCMA on the tumor cells is required for T cell activation and cytotoxicity. These data suggest that BSBM3 can potently and specifically activate T cells in the presence of BCMA+ cells, resulting in specific killing of the target cells.

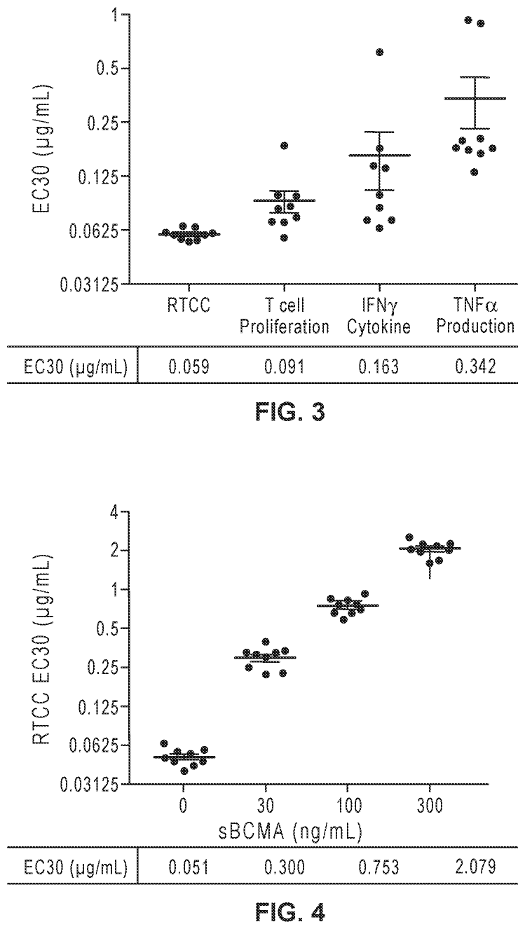

In order to identify the in vitro assay with the most sensitive readout for the activity of BSBM3, the EC30 values were calculated from the different assays, each performed with nine biological replicates. (T-cells from three healthy donors were tested individually each repeated with three independent experiments; FIG. 3). The redirected T-cell cytotoxicity (RTCC) assay which detects specific lysis of MM cells showed the most sensitive and reproducible EC30 values. Therefore, the minimum anticipated biological effect level (MABEL) informing the starting dose was calculated based on EC30 values from RTCC assays.

BCMA has been shown to undergo protease cleavage within its transmembrane domain by γ-secretase, leading to shedding of its extracellular domain as a soluble factor (from here on referred to as soluble BCMA) which serves as a decoy to neutralize its ligand APRIL (Laurent 2015). Average serum levels of soluble BCMA have been reported to be 39 ng/mL in healthy subjects, 89 ng/mL in smoldering myeloma subjects, and 506 ng/mL in newly diagnosed MM subjects (Ghermezi et al., 2017, Haematologica. 102(4): 785-795). Soluble BCMA in the blood and bone marrow of subjects has the potential to bind to and interfere with the activity of BSBM3. As expected, in the presence of 30, 100, or 300 ng/mL soluble BCMA, the EC30 for BSBM3 increased by 6, 15, and 41-fold respectively (FIG. 4). Because 93% of subjects with active and untreated MM have higher than 107.6 ng/mL shed BCMA in their serum (Ghermezi et al., 2017, Haematologica 102(4):785-795), the RTCC assays containing 100 ng/mL soluble BCMA likely better represent the activity of BSBM3 in subjects. Therefore, the EC30 of 0.753 ng/mL (with 100 ng/mL soluble BCMA) was taken into consideration when calculating the starting dose using a MABEL approach. This is consistent with the approach described by Saber et al., 2017, Regul Toxicol Pharmacol; 90:144-152 for CD3-directed bi-specific antibodies.

### 11.4. Example 4

### Non-Clinical Pharmacology (In Vivo)

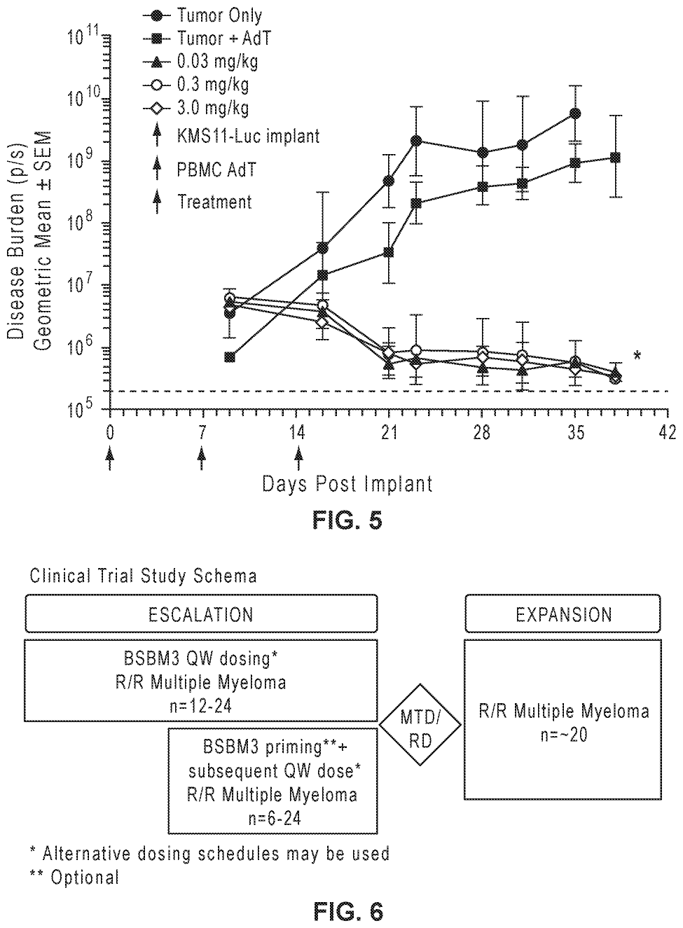

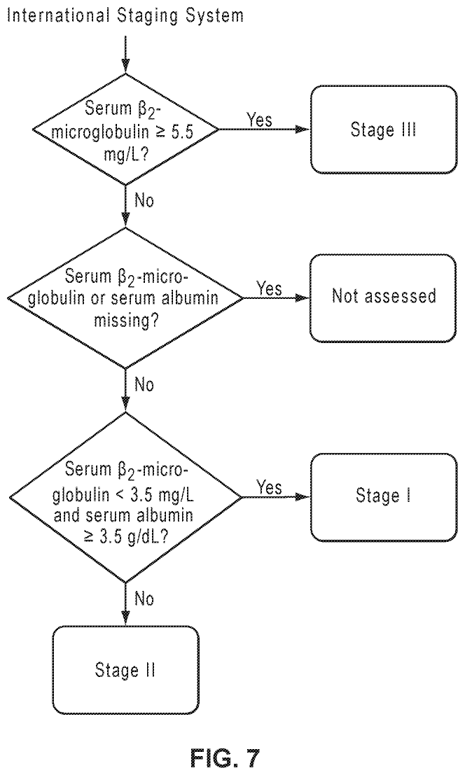

The in vivo activity of BSBM3 was evaluated using the KMS11 xenograft model in immunocompromised NSG mice that had been adoptively transferred with human PBMCs from healthy donors (FIG. 5). KMS11 cells were engineered to overexpress luciferase, which then enabled tumor burden measurement by bioluminescence intensity (BLI). Mice treated with BSBM3 at doses≥0.3 mg/kg showed robust tumor rejection in three independent experiments with PBMCs from two different healthy donors separately (data).

The adoptively transferred model with KMS11 xenograft provided support to the mechanism of action for BSBM3. However, it likely over predicts the anti-MM activity because the adoptively transferred human T cells are hyperactive as indicated by dramatically higher expression of activation markers compared to T cells in donor PBMCs upon isolation (data shown; Ali et al., 2012, PLoS ONE; 7(8): e44219). Therefore the doses that demonstrated anti-MM activity of BSBM3 in this model are not directly translatable to subjects.

### 11.5. Example 5

### Non-Clinical Pharmacokinetics and Metabolism

To investigate the pharmacokinetics (PK) of BSBM3 in a non-binding species, NSG mouse PK studies were performed with and without human peripheral blood mononuclear cells (PBMC). Concentration-time plots showed bi-exponential decline in serum levels, as expected for a monoclonal antibody in non-binding species. In NSG mice humanized with human PBMCs, exposure as measured by AUClast was lower than in PBMC naïve mice. At the last time points, non-linear elimination became apparent indicating expected target mediated drug disposition (TMDD).

BSBM3 binds to both targets (BCMA and CD3) in cynomolgus monkeys, therefore, the toxicokinetic profiles of BSBM3 were investigated in a single dose non-GLP toxicology study (data not shown), and a 4-week GLP toxicology study (data not shown). From the single-dose study, it was determined that exposure to BSBM3, as measured by AUClast increased in a dose proportional manner over the tested doses of 0.3, 1 and 3 mg/kg. Of the five animals dosed, one animal (0.3 mg/kg dose), was confirmed to have anti-drug antibodies (ADA).

In the 4-week cynomolgus monkey GLP toxicology study, animals received 5 weekly i.v. injections of BSBM3 at 1, 3 and 10 mg/kg. After i.v. injection, the maximum exposure to BSBM3 was observed from 0.667 to 4.17 hr post dose, the first time points post-dose. Exposure to BSBM3 as measured by Cmax and AUC0-tau (tau=7 days), increased in an approximately dose-proportional manner over the dose range 1 to 10 mg/kg and was similar in both genders. Accumulation was approximately 1.5 to 1.9-fold (based on AUC0-tau) after 4 weeks of i.v. dosing at all dose levels. No significant gender differences were observed. ADA were detected on Day 28 (1 of 24 treated animals, 1 mg/kg dose) in the main part of the study. During the 6-week recovery part of the study, ADA were detected on Day 57 (1 of 6 treated animals, 3 mg/kg dose) and Day 71 (2 of 6 treated animals, control group). The data suggest that there was likely no significant impact of ADA on TK. ADA were not detected in control animals.

### 11.6. Example 6

### Non-Clinical Toxicology

The safety of BSBM3 was investigated in in vitro and in vivo studies. In vivo studies were conducted in cynomolgus monkeys, which was identified as the pharmacologically relevant species for BSBM3.

Results of safety pharmacology studies indicate the risk of BSBM3 to vital functions of the central nervous system (CNS), respiratory, and cardiovascular system is low. In general, findings from in vivo studies were consistent with BSBM3-related expected pharmacology of peripheral blood, bone marrow and tissue B cell and plasma cell decreases as well as post-dose acute increases of select serum cytokines and more persistent blood and tissue T-cell activation. Depletion of lymphocytes in the gut-associated lymphoid tissue (GALT), lymph nodes and spleen (B cell regions) were noted at all BSBM3 dose levels in the GLP study. During the recovery phase, lymphocyte hyperplasia in these organs was consistent with the regenerative process. In addition, mixed cell immuno-inflammatory lesions were observed in various organs (i.e. gastrointestinal tract (GIT), liver, spleen, heart, kidney, lung) and sometimes associated with infectious agents.

The highest non-severely toxic dose (HNSTD) in the GLP study was identified as 1 mg/kg.

### 11.7. Example 7

### Clinical Study

A clinical trial according according to the schema shown in FIG. 6 is conducted to determine the safety and efficacy of BSBM3 in subjects with multiple myeloma who have received two or more standard of care (SoC) lines of therapy including an IMiD (e.g. lenalidomide or pomalidomide), a proteasome inhibitor (e.g. bortezomib, carfilzomib), and an anti-CD38 agent (e.g. daratumumab) and are relapsed and/or refractory to or intolerant of each regimen.

This study consists of a dose escalation part followed by an expansion part.

This is a FIH, phase I, multicenter, open-label study to determine the safety and efficacy of BSBM3 (a bispecific antibody that specifically binds to BCMA and CD3, as described in throughout the disclosure) in subjects with multiple myeloma who have received two or more standard of care (SoC) lines of therapy including an IMiD (e.g. lenalidomide or pomalidomide), a proteasome inhibitor (e.g. bortezomib, carfilzomib), and an anti-CD38 agent (e.g. daratumumab) and are relapsed and/or refractory to or intolerant of each regimen, with documented evidence of disease progression per International Myeloma Working Group (IMWG) criteria, and who are not eligible for treatment with other regimens known to provide clinical benefit.

11.7.1. Inclusion Criteria

Subjects included in the trial have a confirmed diagnosis of multiple myeloma and have received two or more standard of care (SoC) regimens including an IMiD (e.g. lenalidomide or pomalidomide), a proteasome inhibitor (e.g. bortezomib, carfilzomib), and an anti-CD38 agent (e.g. daratumumab), if available, and are relapsed and/or refractory to or intolerant of each regimen, with documented evidence of disease progression (IMWG criteria) and must not be eligible for treatment with other regimens known to provide clinical benefit, as determined by the investigator (subjects who have received a prior autologous bone marrow transplant, a BCMA CAR-T, or BCMA-ADC therapy and otherwise meet the inclusion criteria are eligible for this study); have an Eastern Cooperative Oncology Group (ECOG) performance status≤2 at screening; and have measurable disease defined by at least 1 of the following 3 measurements: (i) serum M-protein≥1.0 g/dL; (ii) urine M-protein≥200 mg/24 hours; or (iii) serum free light chain (sFLC)>100 mg/L of involved FLC.

11.7.2. Exclusion Criteria

Subjects meeting any of the following criteria are not eligible for inclusion in this study: radiotherapy within 14 days before the first dose of study drug except localized radiation therapy for lytic bone lesions or plasmacytomas; major surgery within 2 weeks before the first dose of study drug; use of systemic chronic steroid therapy (≥10 mg/day of prednisone or equivalent), or any immunosuppressive therapy within 7 days of first dose of study treatment (topical, inhaled, nasal, or ophthalmic steroids are allowed); prior use of BCMA×CD3 bispecific therapies; subjects receiving systemic treatment with any immunosuppressive medication (other than steroids as described above); history of severe hypersensitivity reactions to any ingredient of study drug(s) and other mAbs and/or their excipients; subjects with toxicity to prior BCMA targeted agents; malignant disease, other than that being treated in this study. (Exceptions to this exclusion include the following: malignancies that were treated curatively and have not recurred within 2 years prior to study treatment; completely resected basal cell and squamous cell skin cancers, and completely resected carcinoma in situ of any type.); Active, known or suspected autoimmune disease other than subjects with vitiligo, residual hypothyroidism only requiring hormone replacement, psoriasis not requiring systemic treatment or conditions not expected to recur; subjects who are currently receiving treatment with a prohibited medication that cannot be discontinued at least one week prior to the start of treatment; subjects with Grade≥2 neuropathy, and residual toxic effects from previous therapy must have resolved to Grade≤1 or baseline; subjects with plasma cell leukemia and other plasmacytoid disorders, other than MM; any of the following clinical laboratory results: (i) absolute neutrophil count (ANC)<1,000/mm3 without growth factor support within 7 days prior to the start of treatment; (ii) platelet count<75,000 mm3 without transfusion support within 7 days prior to the start of treatment; (iii) bilirubin>1.5 times the upper limit of the normal range (ULN); (iv) aspartate aminotransferase (AST) or alanine aminotransferase (ALT)>2.5 times the ULN; (v) calculated creatinine clearance<30 ml/min according to Cockcroft-Gault equation; (vi) impaired cardiac function or clinically significant cardiac disease; active infection requiring systemic therapy or other severe infection within 2 weeks before the first dose of study drug; POEMS syndrome (plasma cell dyscrasia with polyneuropathy, organomegaly, endocrinopathy, monoclonal protein, skin changes); prior allogeneic SCT at any time prior to signing informed consent for the study; human immunodeficiency virus (HIV infection); active Hepatitis B (HBV) or Hepatitis C (HCV) infection; use of any live vaccines against infectious diseases (e.g. influenza, varicella, pneumococcus) within 4 weeks of initiation of study treatment; treatment with cytotoxic or small molecule targeted antineoplastics, or any experimental therapy, within 14-days or 5 half-lives whichever is shorter before the first dose of study treatment; initiation of hematopoietic colony-stimulating growth factors (e.g. G-CSF, M-CSF), thrombopoietin mimetics or erythroid stimulating agents ≤2 weeks prior to start of study treatment; intravenous IG infusions given for infection prophylaxis must be discontinued ≥28 days prior to start of study treatment; active central nervous system (CNS) involvement by malignancy or presence of symptomatic CNS metastases, or CNS metastases that require local CNS-directed therapy (such as radiotherapy or surgery), or increasing doses of corticosteroids within the 2 weeks prior to the start of study treatment; serious medical or psychiatric illness likely to interfere with participation in this clinical study; pregnant or nursing (lactating) women, where pregnancy is defined as the state of a female after conception and until the termination of gestation, confirmed by a positive hCG laboratory test; and women of child-bearing potential, defined as all women physiologically capable of becoming pregnant, unless they are using two effective methods of contraception, including at least one highly effective method, at the time of informed consent, during dosing and for 6 months after the last dose of study drug.

11.7.3. Drug Product

Drug product is formulated as Liquid in vial (LIVI) and it is composed of 10 mg/mL BSBM3, 20 mM histidine, 240 mM sucrose, PS20 0.04%, pH 5.5±0.3.

All dosages prescribed and administered to subjects and all dose changes during the study are recorded on the Dosage Administration Record eCRF.

TABLE 3
Investigational drug

| Investigational/ | | | | Fre- | |
|---|---|---|---|---|---|
| Control Drug | | Route of | | quency | |
| (Name and | Pharmaceutical | Admin- | | and/or | Supply |
| Strength) | Dosage Form | istration | Dose | Regimen | Type |
| BSBM3 | Liquid in vial | Intravenous | 3-600 | Weekly | Open |
| 50 mg/5 ml | (LIVI) | use | mcg/ | (QW)* | label |
| | (concentrate for | | kg | | bulk |
| | solution for | | | | supply; |
| | infusion) | | | | vials |

*Alternative dosing regimens may be implemented

Exploration of alternative doses and/or dosing regimens of BSBM3 may be examined in escalation, even after initiation of the expansion part at RD. If enrolling simultaneously, subjects would be assigned in an alternating fashion to cohorts across all the sites in this global study.

11.7.4. Course of Treatment

BSBM3 will be initially administered weekly (Q1W). Study drug treatment will continue until a subject experiences unacceptable toxicity, progressive disease as per IMWG or treatment is discontinued at the discretion of the investigator or the patient. The study design is summarized in FIG. 6. Alternative dosing schedules (e.g. Q2W, Q3W, TIW) may be implemented during the study if supported by emerging data including preliminary PK, PD and efficacy findings from this ongoing trial. If clinically significant cytokine release syndrome (CRS) or associated symptoms are observed during dose escalation, the option of a priming dose may be introduced and subsequent dosing schedules modified.

The design of this phase I, open label study was chosen to characterize the safety and tolerability of BSBM3 in subjects with relapsed and/or refractory multiple myeloma who have been treated with at least 2 prior regimens, and have received an IMiD, proteasome inhibitor, and anti-CD38 antibody (if available), and determine a recommended dose and regimen for future studies. Where necessary, the dose escalation allows the MTD of BSBM3 to be established and will be guided by a Bayesian Logistic Regression Model (BLRM).

The BLRM is a well-established method to estimate the MTD in cancer subjects. The adaptive BLRM will be guided by the escalation with overdose control (EWOC) principle to control the risk of DLT in future subjects on study. The use of Bayesian response adaptive models for small datasets has been accepted by EMEA (“Guideline on clinical trials in small populations”, Feb. 1, 2007) and endorsed by numerous publications (Babb et al., 1998, Stat Med; 17(10):1103-20); (Neuenschwander et al., 2008, Stat Med; 27(13):2420-39); (Neuenschwander et al., 2010, Clin Trials; 7(1):5-18); (Neuenschwander et al., 2014, in A Bayesian Industry Approach to Phase I Combination Trials in Oncology. In Statistical Methods in Drug Combination Studies. Zhao W and Yang H (eds), Chapman & Hall/CRC, 2014), and its development and appropriate use is one aspect of the FDA's Critical Path Initiative.

The decisions on new dose levels are made in a dose escalation meeting based upon the review of subject tolerability and safety information (including the BLRM derived estimates of DLT risk) along with PK, PD and preliminary activity information available at the time of the decision.

11.7.5. Dose Escalation

During dose escalation, subjects with relapsed and/or refractory MM will be treated with BSB3 until the MTD/RD is reached. An estimated 21 subjects are required during escalation to define the MTD/RD.

The safety (including the dose-DLT relationship) and tolerability of the study treatment will be assessed, and regimen(s) and dose(s) will be identified for use in the expansion part based on the review of these data. The RD will also be guided by the available information on PK, PD, and preliminary anti-tumor activity. The dose escalation will be guided by an adaptive Bayesian logistic regression model (BLRM) following the Escalation with Overdose Control (EWOC) principle.

Once the MTD(s)/RD(s) have been determined in the escalation part, additional subjects will be enrolled in the expansion part in order to further characterize the PK, PD, and safety profile of study drug and to assess the preliminary anti-tumor activity of BSBM3. More than one dose level at Q1W schedule might be explored as RDs for expansion. In addition, alternative dosing schedules may be explored in the escalation part. RD(s) of new schedules might be declared.

In the expansion part, subjects with relapsed and/or refractory MM will be treated with BSBM3. The expansion part will enroll approximately 20 subjects. Enrollment may be halted early based on the ongoing review of data from the expansion cohort.

11.7.5.1. Provisional Dosing

The dose for BSBM3 is proposed based on an integrated assessment of predicted pharmacokinetics, the mechanism of action, in vitro potency (to inform a MABEL dosing approach), the impact of circulating BCMA and in vivo safety in the cynomolgus monkey GLP toxicology study. The starting dose for BSBM3 for subjects is 3 mcg/kg administered as a 2 hour intravenous infusion.

The BSBM3 starting dose and the dose levels that may be evaluated during this trial are described in the Table 4. This starting dose is supported by the EC50 value (˜0.07 μg/mL) from the RTCC assay (without added recombinant soluble BCMA) which is believed to represent the most clinically relevant measure of pharmacological activity and is the most sensitive and reproducible assay readout for BSBM3 in vitro (data not shown).

Actual dose levels will be determined based on available toxicity, pharmacokinetic and pharmacodynamic data, guided by the BLRM. Dose escalation will continue until one or more MTDs or RDs are determined.

TABLE 4
Provisional dose levels

| Dose level | Proposed dose* | Increment from previous dose |
|---|---|---|
| −1** | 1 mcg/kg | −150% |
| 1 | 3 mcg/kg | (starting dose) |
| 2 | 6 mcg/kg | 100% |
| 3 | 12 mcg/kg | 100% |
| 4 | 24 mcg/kg | 100% |
| 5 | 48 mcg/kg | 100% |
| 6 | 96 mcg/kg | 100% |
| 7 | 192 mcg/kg | 100% |
| 8 | 384 mcg/kg | 100% |
| 9 | 600 mcg/kg | 56.26% |

*It is possible for additional and/or intermediate dose levels to be added during the course of the study. Cohorts may be added at any dose level below the MTD in order to better understand safety, PK or PD.
**Dose level −1 represents a treatment dose for subjects requiring a dose reduction from the starting dose level. No dose reduction below dose level −1 is permitted for this study.

As an option, a priming dose will be used if, during dose escalation, 2 patients experience an event of Grade≥3 infusion related reaction (IRR) or cytokine release syndrome (CRS) that does not resolve to Grade or baseline within 48 hours.

The priming dose will be selected at a dose level determined to be safe (the dose will be at least one dose level lower than the maximum dose tested in the previous cohorts and meeting the EWOC criteria). In addition, as an added safety measure, one third of the priming dose will be given on Day 1 and two thirds of the dose on Day 2. Once the priming dose level is determined, the dose levels that may be evaluated in the subsequent cohorts are defined relative to the priming dose and are listed in Table 5. For example, if the priming dose is defined to be 100 mcg/kg (i.e. dose level X in Table 5), the dose on Day 1 will be 33.33 mcg/kg and on Day 2 will be 66.66 mcg/kg. The third and subsequent infusions (on Day 8, 15 and 22) will be at 200 mcg/kg (i.e. dose level X+1, where X+1 is the next provisional dose level after X listed in Table 3). Actual dose levels will be determined based on available toxicity, pharmacokinetic and pharmacodynamic data. A separate BHLRM will be constructed to guide the dose escalation with EWOC criteria. Dose escalation will continue until one or more MTDs or RDs are determined.

TABLE 5
Provisional dose levels with priming dose

| | | Day2^{¥} | | | |
|---|---|---|---|---|---|
| | Day 1^{¥} | (priming | | | |
| Cohort* | (priming dose) | dose) | Day 8^{£} | Day 15 | D22 |
| P-1** | X*1/3 | X*2/3 | X | X + 1 | X + 1 |
| P1 | X*1/3 | X*2/3 | X + 1 | X + 1 | X + 1 |
| P2 | X*1/3 | X*2/3 | X + 2 | X + 2 | X + 2 |
| P3 | X*1/3 | X*2/3 | X + 3 | X + 3 | X + 3 |

^{¥}The priming dose will be split into 2 days. On Day 1, subject will receive ⅓ of the total priming dose X and on Day 2, the rest of ⅔ of the priming dose will be administered.
^{£}The “X + 1/+2/+3” dose levels refer to 1/2/3 dose levels higher than X according to the provisional dose table.
*It is possible for additional and/or intermediate dose levels to be added during the course of the study. Cohorts may be added at any dose level below the MTD in order to better understand safety, PK or PD.
**Cohort P-1 represent treatment doses for subjects requiring a dose reduction from the priming dose level where the dose on Day 8 does not escalate but stay the same as the total priming dose. No dose reduction below cohort P-1 is permitted for this study.

The priming dose level may be adapted if needed, in accordance with evolving trial safety and tolerability findings.

11.7.5.2. Guidelines for Dose Escalation and Determination of MTD/RD

The dose escalation is conducted in order to establish the dose(s) of BSBM3 to be used in the expansion part. Specifically, it is the one or more doses that is believed to have the most appropriate benefit-risk as assessed by the review of safety, tolerability, PK, any available efficacy, and PD, taking into consideration the maximum tolerated dose (MTD).

The MTD is the highest dose estimated to have less than 25% risk of causing a dose-limiting toxicity (DLT) during the DLT evaluation period in more than 33% of treated subjects. The dose(s) selected for the expansion part can be any dose equal to or less than the MTD, and may be declared without identifying the MTD.

Each dose escalation cohort will start with 1 to 6 newly treated subjects. They must have adequate exposure and follow-up to be considered evaluable for dose escalation decisions.

If any subject experiences a DLT during the DLT evaluation period, the minimum cohort size will be increased to three.

If one or more subjects discontinue and fail to meet the evaluability criteria, the replacement policy may be used to enroll additional subjects to the same cohort, in order to support the benefit-risk assessment.

The treatment period will begin on Cycle 1 Day 1. For the purpose of scheduling and evaluations, a treatment cycle will consist of 28 days. For each cohort where the dose level on Cycle 1 Day 1 is higher than any dose previously tested and shown to be safe, a staggered approach for the first two subjects in a cohort will be utilized. Following dosing of the first subject, the next subject will be dosed a minimum of 72 hours after the previous subject is dosed. Following completion of this staggered dosing of the first two subjects, subsequent subjects will be treated without staggering, however, no more than 1 patient within a cohort will have their first infusion on any given day. Dose escalation decisions will be made when all subjects in a cohort have completed the DLT evaluation period or discontinued. Decisions will be based on a synthesis of all relevant data available from all dose levels evaluated in the ongoing study, including safety information, available PK, available PD and preliminary efficacy.

Any dose escalation decisions will not exceed the dose level satisfying the EWOC principle by the Bayesian logistic regression model (BLRM). For any dose levels, the dose for the next escalation cohort will not exceed a 100% increase from the previously tested safe dose. Smaller increases in dose may be recommended by the Investigators and Sponsor upon consideration of all of the available clinical data.

To better understand the safety, tolerability, PK, PD or anti-tumor activity of BSBM3 before or while proceeding with further escalation, enrichment cohorts of 1 to 6 subjects may be enrolled at any dose level at or below the highest dose previously tested and shown to be safe.

To reduce the risk of exposing subjects to an overly toxic dose, if 2 subjects experience a DLT in a new cohort, the BLRM will be updated with the most up-to-date new information from all cohorts, without waiting for all subjects from the current cohort to complete the evaluation period.
If the 2 DLTs occur in an escalation cohort, enrollment to that cohort will stop, and the next cohort will be opened at a lower dose level that satisfies the EWOC criteria.
If the 2 DLTs occur in an enrichment cohort, then upon re-evaluation of all relevant data, additional subjects may be enrolled into the open cohorts only if the dose still meets the EWOC criteria. Alternatively, if recruitment to the same dose cannot continue, a new cohort of subjects may be recruited to a lower dose that satisfies the EWOC criteria. Additionally, if 2 or more patients experience a DLT in a dosing cohort, the next dose-escalation level will not be more than 50% above the previous dose level.

11.7.6. Management of CRS

At least 2 doses of tocilizumab per patient are available on site prior to infusion of BSBM3. Hospitals should have timely access to additional doses of tocilizumab. Supportive care, tocilizumab, and corticosteroids have been used for effective management of CRS. Prompt responses to tocilizumab have been seen in most subjects.

Cytokine release syndrome (CRS) is identified based on clinical presentation (see Table 6). Other causes of fever, hypoxia, and hypotension are evaluated for and treated, and subjects are monitored for signs or symptoms of CRS for at least 4 weeks after treatment with BSBM3. Subjects are counseled to seek immediate medical attention should signs or symptoms of CRS occur at any time.

At the first sign of CRS (see Table 6), the patient is immediately evaluated for hospitalization and treatment with supportive care, tocilizumab and/or corticosteroids is instituted as indicated.

A recommended treatment algorithm for the management of CRS is presented below in Table 7 and Table 8. The CRS management algorithm is a guideline and the investigator may use discretion or modify the treatment approach as needed for an individual subject.

TABLE 6
Clinical signs and symptoms associated with CRS
(Lee et al., 2014, Blood 124(2): 188-95)

| Organ system | Symptoms |
|---|---|
| Constitutional | Fever ± rigors, malaise, fatigue, anorexia, myalgia, arthralgia, nausea, vomiting, |
| | headache |
| Skin | Rash |
| Gastrointestinal | Nausea, vomiting, diarrhea |
| Respiratory | Tachypnea, hypoxemia |
| Cardiovascular | Tachycardia, widened pulse pressure, hypotension, increased cardiac output |
| | (early), potentially diminished cardiac output (late) |
| Coagulation | Elevated D-dimer, hypofibrinogenemia ± bleeding |
| Renal | Azotemia |
| Hepatic | Transaminitis, hyperbilirubinemia |
| Neurologic | Headache, mental status changes, confusion, delirium, word finding difficulty or |
| | frank aphasia, hallucinations, tremor, dysmetria, altered gait, seizures |

TABLE 7
CRS Management

| | Symptomatic | | |
|---|---|---|---|
| CRS severity | treatment | Tocilizumab | Corticosteroids |
| Mild symptoms requiring | Exclude other causes | Not applicable | Not applicable |
| symptomatic treatment only | (e.g. infection) and | | |
| e.g. low fever, fatigue, | treat specific | | |
| anorexia, etc. | symptoms with e.g. | | |
| | antipyretics, anti- | | |
| | emetics, anti- | | |
| | analgesics, etc. | | |
| | If neutropenic, | | |
| | administer antibiotics | | |
| | per local guidelines | | |
| Symptoms requiring | Antipyretics, oxygen, | If no improvement | If no improvement |
| moderate intervention: | intravenous fluids | after symptomatic | within 12-18 hours |
| high fever | and/or low dose | treatment administer | of tocilizumab, |
| hypoxia | vasopressors as | tocilizumab i.v. over 1 | administer a daily |
| mild hypotension | needed. | hour: | dose of 2 mg/kg i.v. |
| | | 8 mg/kg (max 800 | methylprednisolone |
| Symptoms requiring | High-flow oxygen | mg) | (or equivalent) until |
| aggressive intervention: | Intravenous fluids and | if body weight ≥30 | vasopressor and |
| hypoxia requiring high- | high-dose | kg | oxygen no longer |
| flow oxygen | vasopressor/s | 12 mg/kg if body | need, then taper.* |
| supplementation or | Treat other organ | weight | |
| hypotension requiring | toxicities as per local | <30 kg | |
| high-dose or multiple | guidelines | If no improvement, | |
| vasopressors | | repeat every 8 hours | |
| Life-threatening symptoms: | Mechanical ventilation | (max total of 4 doses)* | |
| hemodynamic | Intravenous fluids and | | |
| instability despite i.v. | high-dose | | |
| fluids and vasopressors | vasopressor/s | | |
| worsening respiratory | Treat other organ | | |
| distress | toxicities as per local | | |
| rapid clinical | guidelines | | |
| Deterioration | | | |

* If no improvement after tocilizumab and steroids, other anti-cytokine and anti-T-cell therapies are considered. These therapies may include siltuximab (11 mg/kg i.v. over 1 hour), high doses of steroids (e.g. high dose methylprednisolone or equivalent steroid dose according to local ICU practice) cyclophosphamide, anti-thymocyte globulin (ATG) or alemtuzumab.

TABLE 8
High Dose Vasopressors

| Vasopressor | Dose to be given for ≥3 hours |
|---|---|
| Norepinephrine monotherapy | ≥20 mcg/min |
| Dopamine monotherapy | ≥10 mcg/kg/min |
| Phenylephrine monotherapy | ≥200 mcg/min |
| Epinephrine monotherapy | ≥10 mcg/min |
| If on vasopressin | Vasopressin + norepinephrine |
| | equivalent (NE) of ≥10 mcg/min* |
| If on combination vasopressors | NE of ≥0 mcg/min* |
| (not vasopressin) | |

*Vasopressin and Septic Shock Trial (VASST) Norepinephrine Equivalent Equation:
NE dose = [norepinephrine (mcg/min)] + [dopamine (mcg/kg/min) ÷ 2] + [epinephrine (mcg/min)] + [phenylephrine (mcg/min) + 10] (Russell et al., 2008, N Engl J Med; 358(9):877-87)

Other anti-cytokine therapies may also be considered upon their availability, if the subject does not respond to tocilizumab. If the subject experiences ongoing CRS despite administration of anti-cytokine directed therapies, anti-T-cell therapies such as cyclophosphamide, anti-thymocyte globulin (ATG) or alemtuzumab may be considered. These therapies are captured in appropriate CRFs.

The management of CRS is based solely upon clinical parameters as described in Table 7. Ferritin, CRP and serum cytokine levels are not be used for clinical management decisions. Cases of transient left ventricular dysfunction, as assessed by echocardiogram (ECHO), have been reported in some subjects with severe (Grade 4) CRS. Therefore consideration is given to monitoring cardiac function by ECHO during severe CRS, especially in cases with prolonged severe hemodynamic instability, delayed response to high dose vasopressors, and/or severe fluid overload.

11.7.7. Primary Endpoints

The primary endpoints of the study are set forth in Table 9.

TABLE 9
Primary endpoints

| Objective | Primary endpoint |
|---|---|
| Safety | Incidence and severity of AEs and SAEs, |
| | including changes in laboratory values, vital |
| | signs, ECGs, and CRS/immune-mediated |
| | reactions |
| Tolerability | Dose interruptions, reductions, and dose intensity |
| Identification of | Incidence of dose limiting toxicities (DLTs) in |
| recommended dose | Cycle 1 |

A dose-limiting toxicity (DLT) is defined as an adverse event or abnormal laboratory value assessed as clinically relevant, occurring 28 days following the first administration of study treatment.

11.7.8. Results

BSBM3 is found to be safe and well tolerated, and found to have anti-tumor activity.

### 11.8. Example 8

### Effective Dose Range of AL-102 for BCMA Shedding Inhibition

### 11.8.1. Overview

Gamma secretase inhibitor (GSI), AL-102, was evaluated for its effect on B cell maturation antigen (BCMA) shedding in KMS11 cells in vitro.

### 11.8.2. Materials and Methods

### 11.8.2.1. GSI Treatment of KMS11 Cells

KMS11-Luc cells were cultured in a 96-well round bottomed plate (Corning #3799) at 1.5×10⁵ cells per well in a final volume of 200 μL that included a 12-point, 5-fold serial dilution of AL-102 in RPMI1640 (Gibco #11875-085) supplemented with 20% FBS (Seradigm #1500-500) and L-glutamine (Thermo Fisher #25030-081). The highest starting concentration of AL-102 was 1 μM. Cells were incubated for 20 hours at 37° C./5% CO2. Cells were pelleted, supernatant collected for measurement of shed BCMA levels, and cell pellets stained for evaluation of BCMA membrane expression levels.

### 11.8.2.2. Measurement of Shed BCMA Levels by ELISA

Soluble BCMA levels in supernatant were determined by ELISA following a vendor supplied protocol (R&D Systems #DY193). Briefly, recombinant human BCMA-Fc protein was included in the kit, and used to generate a standard curve. Collected samples were assayed and sBCMA concentrations extrapolated from the standard curve. Quantified values as determined by the kit were divided by 5.5 to correct for a molecular mass difference between BCMA-Fc fusion protein used in the kit as a standard curve (32,554.6 Da) and the mass of endogenously shed BCMA extra-cellular domain (5,899.3 Da). The results were analyzed using SoftMax Pro v5.4.1 and graphed in GraphPad Prism.

### 11.8.2.3. Analysis of BCMA Membrane Expression by Flow Cytometry

Cells were pelleted by centrifugation, and supernatants were transferred to a fresh plate and frozen at −80° C. for later sBCMA analysis by ELISA. For membrane BCMA analysis, cell pellets were resuspended in 100 μL BD Stain Buffer containing BSA (BD#554657) and stained with anti-BCMA-PE (Biolegend, clone 19F2 1.25 ul/test) and Fixable Viability Dye eFluor506 (Thermo Scientific, 1:800 dilution) for 30 minutes at 4° C. Samples were analyzed by flow cytometry on a BD LSR Fortessa instrument. FlowJo v10 software was used for analysis. The anti-BCMA antibody binding capacity (ABC) on KMS11 cells was determined using Quantum Simply Cellular beads (Bangs Laboratories) following a vendor supplied protocol. The ABC is an estimate of the quantity of receptors per cell. These results were plotted in Graphpad Prism against the concentration of AL-102.

### 11.8.3. Results

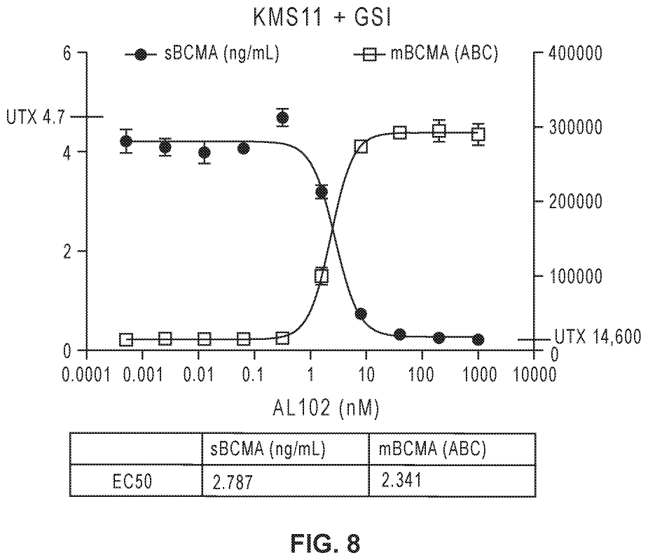

AL-102 effectively inhibited shedding of BCMA from KMS11 cells in a dose-dependent manner, which resulted in increased BCMA expression on the cell surface over the same effective dose range (FIG. 8). Untreated KMS11 cells have a BCMA antibody-binding capacity (ABC) of ˜14,000. The average ABC with treatment of 1 μM AL-102 was ˜285,000, a 20-fold increase in cell surface BCMA expression with AL-102 treatment.

### 11.9. Example 9

### AL-102 Impact on BSBM3 Potency

### 11.9.1. Overview

To evaluate the ability of AL-102 to enhance the activity of BSBM3, a redirected T cell cytotoxicity (RTCC) assay was performed using human T cells and a BCMA-expressing multiple myeloma cell line treated with dose range combinations of BSBM3 and AL-102 in a 10×8 matrix fashion.

### 11.9.2. Materials and Methods

### 11.9.2.1. Healthy Human T Cell Isolation

Human T cells were enriched from peripheral blood of three healthy human donors. First, peripheral blood mononuclear cells (PBMCs) were fractionated from donor blood using a Ficoll-Paque PLUS density gradient (GE Healthcare #17-1440-02) in Leucosep tubes (Greiner #227290) and stored as viable frozen aliquots in liquid nitrogen. PBMC were thawed and Pan T cells were isolated by negative selection according to manufacturer's recommended protocol (Miltenyi #130-096-535). The unlabeled cell fraction, enriched for T cells, was collected by manual magnetic separation on LS columns (Miltenyi #130-042-401). T cells were prepared in T cell media (TCM) consisting of RPMI-1640 (Gibco #11875-085), 10% FBS (Seradigm #1500-500), 1% Pen/Strep (Life Technologies #15070063), 1% L-glutamine (Thermo Scientific #25030-081), 1% Non-Essential Amino Acids (NEAA) (Life Technologies #11140-050), Sodium Pyruvate (NaPy) (Life Technologies #11360-070), HEPES (Life technologies, Cat #15630080), 0.1% 2-Betamercaptoethanol (2-BME) (Life Technologies, Cat #21985-023).

### 11.9.2.2. KMS11 Multiple Myeloma Cell Line

KMS11 multiple myeloma cell line was cultured in RPMI1640 supplemented with 20% FBS (Gibco #11875-085, Seradigm #1500-500).

### 11.9.2.3. Re-Directed T Cell Cytotoxicity (RTCC) Assay

The target MM cell line KMS11 was transduced to constitutively express luciferase (KMS11-Luc), and used to measure cell viability/survival. KMS11-Luc cells were pelleted and resuspended in fresh media immediately prior to plating to remove any basal level of shed BCMA that may be present. 7,500 KMS11-Luc target cells in 10 μL TCM were added to wells of 384-well plates (Corning #3765). 10 μl of BSBM3 at a concentration of 10 nM was serially diluted 5-fold, and 10 μl of AL-102 at a concentration of 1000 nM was serially diluted 5-fold, and dispensed into corresponding wells of the assay plates. 15,000 T cells were added to corresponding wells of the assay plate in 10 μL TCM for an E:T of 2:1. The assay was incubated at 37° C./5% CO2 for 48 hr, followed by measurement of luciferase activity to indicate target cell viability (BrightGlo, Promega #E2650) following manufacturer's protocols. Plates were read on an Envision plate reader. Target cells only (KMS11-Luc) without T cells or antibodies served as control and represent 100% luciferase activity (100% viability). Data were plotted and analyzed using GraphPad Prism. EC50 values were calculated using sigmoidal, 4-parameter non-linear regression curve fit.

### 11.9.3. Results

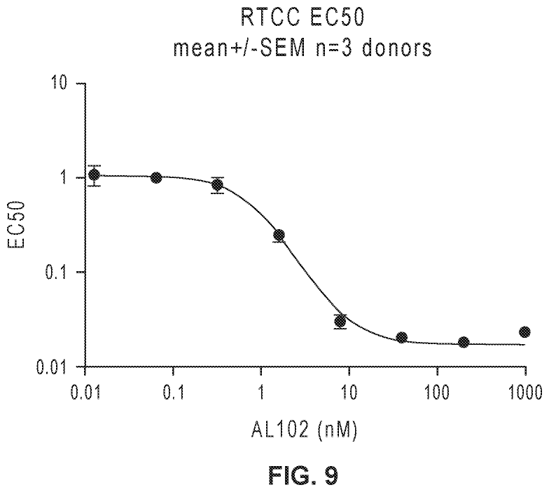

An RTCC assay was set up with three individual T cell donors cultured with KMS11-Luc cells in the presence of dose response curves of BSBM3 alone or in combination with AL-102. BSBM3 showed a dose dependent effect on KMS11-Luc cell death with EC50 value of 1 nM. Combination of BSBM3 with AL-102 increased BSBM3 killing capacity (FIG. 9). AL-102 decreased BSBM3 EC50 values from 1 nM to as low as 0.02 nM, which indicates enhanced RTCC activity of BSBM3 in the presence of AL-102. This represents a 50-fold increase in BSBM3 potency. AL-102 at 0.32 nM or lower had minimal effect on BSBM3 potency, at 1.6 nM had a moderate effect, and at concentrations of 8 nM or higher showed maximum enhancement of BSBM3 potency. These results demonstrated that AL-102 combination with BSBM3 synergistically enhanced the RTCC potency of BSBM3.

What is claimed is:

1. A method of treating a subject suffering from multiple myeloma, comprising administering to the subject one or more treatment doses of a bispecific antibody that binds to human BCMA and human CD3 and comprises:
(a) a first polypeptide whose amino acid sequence comprises the amino acid sequence of SEQ ID NO:1;
(b) a second polypeptide whose amino acid sequence comprises the amino acid sequence of SEQ ID NO:2; and
(c) a third polypeptide whose amino acid sequence comprises the amino acid sequence of SEQ ID NO:3.

2. The method of claim 1, wherein the subject has measurable disease.

3. The method of claim 2, wherein the subject has serum M-protein levels of ≥1 g/dL.

4. The method of claim 2 or claim 3, wherein the subject produces urine M-protein levels of ≥200 mg/24 hours.

5. The method of any one of claims 2 to 4, wherein the subject has serum free light chain (sFLC) levels of at least 100 mg/L of involved FLC.

6. The method of any one of claims 1 to 5 wherein the multiple myeloma is relapsed.

7. The method of any one of claims 1 to 6, wherein the multiple myeloma is refractory.

8. The method of any one of claims 1 to 7, wherein the bispecific antibody is administered to the subject intravenously.

9. The method of claim 8, wherein the bispecific antibody is administered to the subject as an infusion.

10. The method of claim 9, wherein the infusion is over a 1.5-3 hour span.

11. The method of claim 9, wherein the infusion is over a 2 hour span.

12. The method of any one of claims 1 to 11, wherein one or more treatment doses are administered weekly.

13. The method of any one of claims 1 to 12, wherein the subject receives at least three treatment doses.

14. The method of claim 13, wherein the three treatment doses are administered over a period of less than one month.

15. The method of claim 14, wherein the three treatment doses are administered over a period of 14 or 15 days.

16. The method of any one of claims 1 to 12, wherein the subject receives at least four treatment doses.

17. The method of claim 16, wherein the four treatment doses are administered over a period of less than one month.

18. The method of claim 17, wherein the four treatment doses are administered over a period of 21, 22 or 23 days.

19. The method of any one of claims 1 to 18, wherein one or more treatment doses or each treatment dose ranges from:
(a) about 1 μg/kg to about 1200 μg/kg; or
(b) about 50 μg to about 96 mg.

20. The method of claim 19, wherein one or more treatment doses or each treatment dose ranges from:
(a) about 3 μg/kg to about 600 μg/kg; or
(b) about 150 μg to about 48 mg.

21. The method of claim 19, wherein one or more treatment doses or each treatment dose ranges from:
(a) about 5 μg/kg to about 100 μg/kg; or
(b) about 150 μg to about 8 mg.

22. The method of claim 19, wherein one or more treatment doses or each treatment dose ranges from:
(a) about 10 μg/kg to about 200 μg/kg; or
(b) about 500 μg to about 16 mg.

23. The method of claim 19, wherein one or more treatment doses or each treatment dose ranges from:
(a) about 50 μg/kg to about 400 μg/kg; or
(b) about 2.5 mg to about 32 mg.

24. The method of claim 19, wherein one or more treatment doses or each treatment dose ranges from:
(a) about 100 μg/kg to about 600 μg/kg; or
(b) about 5 mg to about 96 mg.

25. The method of claim 19, wherein one or more treatment doses or the first treatment dose is:
(a) about 1 μg/kg; or
(b) about 50 μg to about 80 μg.

26. The method of claim 19, wherein one or more treatment doses or the first treatment dose is:
(a) about 3 μg/kg; or
(b) about 150 μg to about 240 μg.

27. The method of claim 19, wherein one or more treatment doses or the first treatment dose is:
(a) about 6 μg/kg; or
(b) about 300 μg to about 480 μg.

28. The method of claim 19, wherein one or more treatment doses or the first treatment dose is:
(a) about 12 μg/kg; or
(b) about 600 μg to about 960 μg.

29. The method of claim 19, wherein one or more treatment doses or the first treatment dose is:
(a) about 24 μg/kg; or
(b) about 1.2 mg to about 1.92 mg.

30. The method of claim 19, wherein one or more treatment doses or the first treatment dose is:
(a) about 48 μg/kg; or
(b) about 2.4 mg to about 3.84 mg.

31. The method of claim 19, wherein one or more treatment doses or the first treatment dose is:
(a) about 96 μg/kg; or
(b) about 4.8 mg to about 7.68 mg.

32. The method of claim 19, wherein one or more treatment doses or the first treatment dose is:
(a) about 192 μg/kg; or
(b) about 9.6 mg to about 15.36 mg.

33. The method of claim 19, wherein one or more treatment doses or the first treatment dose is:
(a) about 384 μg/kg; or
(b) about 19.2 mg to about 30.72 mg.

34. The method of claim 19, wherein one or more treatment doses or the first treatment dose is:
(a) about 600 μg/kg; or
(b) about 30 mg to about 48 mg.

35. The method of any one of claims 1 to 34, wherein administering a treatment dose of the bispecific antibody comprises:
(a) administering a first treatment dose; and
(b) escalating the treatment dose to a final treatment dose.

36. The method of claim 35, which comprises administering a second treatment dose that is the same as the first treatment dose prior to escalating the treatment dose.

37. The method of claim 35 or claim 36, wherein step (b) comprises escalating the treatment dose more than once.

38. The method of any one of claims 35 to 37, wherein each dose escalation results in no more than doubling of the preceding dose.

39. The method of any one of claims 35 to 38, wherein the first treatment dose ranges from:
(a) about 1 μg/kg to about 6 μg/kg; or
(b) about 50 μg to about 480 μg.

40. The method of any one of claims 35 to 38, wherein the first treatment dose is:
(a) about 1 μg/kg; or
(b) about 50 μg to about 80 μg.

41. The method of any one of claims 35 to 38, wherein the first treatment dose is:
(a) about 3 μg/kg; or
(b) about 150 μg to about 240 μg.

42. The method of any one of claims 35 to 38, wherein the first treatment dose is:
(a) about 6 μg/kg; or
(b) about 300 μg to about 480 μg.

43. The method of any one of claims 35 to 38, wherein the final treatment dose ranges from:
(a) about 5 μg/kg to about 600 μg/kg; or
(b) about 150 μg to about 48 mg.

44. The method of any one of claims 35 to 38, wherein the final treatment dose ranges from:
(a) about 10 μg/kg to about 200 μg/kg; or
(b) about 500 μg to about 16 mg.

45. The method of any one of claims 35 to 38, wherein the final treatment dose ranges from:
(a) about 50 μg/kg to about 400 μg/kg; or
(b) about 2.5 mg to about 32 mg.

46. The method of any one of claims 35 to 38, wherein the final treatment dose ranges from:
(a) about 100 μg/kg to about 600 μg/kg; or
(b) about 5 mg to about 96 mg.

47. The method of any one of claims 35 to 38, wherein the final treatment dose is:
(a) about 6 μg/kg; or
(b) about 300 μg to about 480 μg.

48. The method of any one of claims 35 to 38, wherein the final treatment dose is:
(a) about 12 μg/kg; or
(b) about 600 μg to about 960 μg.

49. The method of any one of claims 35 to 38, wherein the final treatment dose is:
(a) about 24 μg/kg; or
(b) about 1.2 mg to about 1.92 mg.

50. The method of any one of claims 35 to 38, wherein the final treatment dose is:
(a) about 48 μg/kg; or
(b) about 2.4 mg to about 3.84 mg.

51. The method of any one of claims 35 to 38, wherein the final treatment dose is:
(a) about 96 μg/kg; or
(b) about 4.8 mg to about 7.68 mg.

52. The method of any one of claims 35 to 38, wherein the final treatment dose is:
(a) about 192 μg/kg; or
(b) about 9.6 mg to about 15.36 mg.

53. The method of any one of claims 35 to 38, wherein the final treatment dose is:
(a) about 384 μg/kg; or
(b) about 19.2 mg to about 30.72 mg.

54. The method of any one of claims 35 to 38, wherein the final treatment dose is:
(a) about 600 μg/kg; or
(b) about 30 mg to about 48 mg.

55. The method of any one of claims 1 to 54 which comprises, prior to administering the first treatment dose of the bispecific antibody, administering a priming dose of the bispecific antibody to the subject.

56. The method of claim 55, wherein the priming dose is less than the first treatment dose.

57. The method of claim 55, wherein the priming dose is equal to the first treatment dose.

58. The method of any one of claims 55 to 57, wherein administration of the priming dose is initiated one week prior to administering the first treatment dose.

59. The method of claim 55 or claim 58, wherein the priming dose is divided.

60. The method of claim 59, wherein the priming dose is administered over a period of two days.

61. The method of claim 60, wherein less than half the priming dose is administered on the first day and the remainder of the priming dose is administered on the second day.

62. The method of claim 61, wherein about a third of the priming dose is administered on the first day and about two thirds of the priming dose is administered on the second day.

63. The method of any one of claims 55 to 62, wherein the priming dose ranges from:
(a) about 0.5 μg/kg to about 6 μg/kg; or
(b) about 25 μg to about 480 μg.

64. The method of claim 63, wherein the priming dose is:
(a) about 1 μg/kg; or
(b) about 50 μg to about 80 μg.

65. The method of claim 63, wherein the priming dose is:
(a) about 2 μg/kg; or
(b) about 100 μg to about 160 μg.

66. The method of claim 63, wherein the priming dose is:
(a) about 3 μg/kg; or
(b) about 150 μg to about 240 μg.

67. The method of claim 63, wherein the priming dose is:
(a) about 4 μg/kg; or
(b) about 200 μg to about 320 μg.

68. The method of claim 63, wherein the priming dose is:
(a) about 5 μg/kg; or
(b) about 250 μg to about 400 μg.

69. The method of claim 63, wherein the priming dose is:
(a) about 6 μg/kg; or
(b) about 300 μg to about 480 μg.

70. The method of any one of claims 1 to 34, which comprises
(a) administering one third of a priming dose of the bispecific antibody to the subject on day 1 of the treatment;
(b) administering two thirds of the priming dose of the bispecific antibody to the subject on day 2 of the treatment;
(c) administering a first treatment dose to the subject on one of days 5-11 of the treatment;
(d) administering a second treatment dose to the subject on one of days 12-18 of the treatment; and
(e) administering a third treatment dose to the subject one of days 19-25 of the treatment.

71. The method of claim 70, which comprises
(a) administering one third of the priming dose of the bispecific antibody to the subject on day 1 of the treatment;
(b) administering two thirds of the priming dose of the bispecific antibody to the subject on day 2 of the treatment;
(c) administering a first treatment dose to the subject on one of days 6-10 of the treatment;
(d) administering a second treatment dose to the subject on one of days 13-17 of the treatment; and
(e) administering a third treatment dose to the subject on one of days 20-24 of the treatment.

72. The method of claim 70, which comprises
(a) administering one third of the priming dose of the bispecific antibody to the subject on day 1 of the treatment;
(b) administering two thirds of the priming dose of the bispecific antibody to the subject on day 2 of the treatment;
(c) administering a first treatment dose to the subject on one of days 7-9 of the treatment;
(d) administering a second treatment dose to the subject on one of days 14-16 of the treatment; and
(e) administering a third treatment dose to the subject on one of days 21-23 of the treatment.

73. The method of claim 70, which comprises
(a) administering one third of a priming dose of the bispecific antibody to the subject on day 1 of the treatment;
(b) administering two thirds of the priming dose of the bispecific antibody to the subject on day 2 of the treatment;
(c) administering a first treatment dose to the subject on day 8 of the treatment;
(d) administering a second treatment dose to the subject on day 15 of the treatment; and
(e) administering a third treatment dose to the subject on day 22 of the treatment.

74. The method of any one of claims 70 to 73, wherein the priming dose is the same as the first treatment dose.

75. The method of any one of claims 70 to 73, wherein the first treatment dose is 2 to 8 times the priming dose.

76. The method of any one of claims 70 to 73, wherein the first treatment dose is 2 times the priming dose.

77. The method of any one of claims 70 to 73, wherein the first treatment dose is 4 times the priming dose.

78. The method of any one of claims 70 to 73, wherein the first treatment dose is 8 times the priming dose.

79. The method of any one of claims 70 to 78, wherein the second treatment dose is equal to the first treatment dose.

80. The method of any one of claims 70 to 79, wherein the third treatment dose is the same as the first treatment dose.

81. The method of any one of claims 1 to 80, which further comprises administering to the subject one or more agents that reduces a side effect of the bispecific antibody.

82. The method of claim 81, wherein the agent is administered prior to, concurrently with, or after initiating treatment with the bispecific antibody, or any combination of the foregoing.

83. The method of claim 81 or claim 82, wherein the side effect is cytokine release syndrome (CRS).

84. The method of claim 83, wherein the one or more agents reduce the onset or severity of CRS.

85. The method of any one of claims 81 to 84, wherein the one or more agents comprise a glucocorticoid.

86. The method of claim 85, wherein the glucocorticoid is methylprednisolone.

87. The method of claim 86, wherein the methylprednisolone is given at a dose of least 2 mg/kg.

88. The method of any one of claims 81 to 83, wherein the one or more agents comprise paracetamol, acetaminophen, antihistamines, steroids, anti-T cell directed therapy, or any combination thereof.

89. The method of claim 88, wherein the one or more agents comprise an anti-T cell directed therapy that is tocilizumab, canakinumab, or any combination thereof.

90. The method of any one of claims 1 to 89, which further comprises administering a second therapeutic agent to the subject.

91. The method of claim 90, wherein the bispecific antibody and the second therapeutic agent are administered simultaneously, separately, or over a period of time.

92. The method of claim 90 or 91, wherein the second therapeutic agent is a gamma secretase inhibitor (GSI).

93. The method of claim 92, wherein the GSI is LY-450139, PF-5212362, BMS-708163, MK-0752, ELN-318463, BMS-299897, LY-411575, DAPT, AL-101 (BMS-906024), AL-102 (BMS-986115), PF-3084014, RO4929097, or LY3039478.

94. The method of claim 93, wherein the GSI is AL-102.

95. The method of any one of claims 92 to 94, wherein the GSI is administered orally.

96. The method of any one of claims 92 to 95, wherein the GSI is administered prior to administration of the bispecific antibody.

97. The method of claim 90 or 91, wherein the second therapeutic agent is an immunomodulator.

98. The method of claim 90 or 91, wherein the second therapeutic agent is an immune checkpoint inhibitor.

99. The method of claim 90 or 91, wherein the second therapeutic agent is a TIM-3 inhibitor.

100. The method of claim 99, wherein the TIM-3 inhibitor is MBG453.

101. The method of claim 90 or 91, wherein the second therapeutic agent is a LAG-3 inhibitor.

102. The method of claim 101, wherein the LAG-3 inhibitor is LAG525.

103. The method of claim 90 or 91, wherein the second therapeutic agent is a PD-1 inhibitor.

104. The method of claim 103, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

105. The method of claim 103 or 104, wherein the PD-1 inhibitor is PDR001.

106. The method of claim 104 or 105, wherein the PD-1 inhibitor is administered at a dose of about 100 mg once every four weeks, or about 200 mg once every four weeks, or about 300 mg once every four weeks, or about 400 mg once every four weeks, or about 500 mg once every four weeks.

107. The method of claim 106, wherein the PD-1 inhibitor is administered at a dose of about 400 mg once every four weeks.

108. The method of any one of claims 90 to 107, wherein the second therapeutic agent is administered intravenously.

109. The method of any one of claims 1 to 108, wherein the subject has been previously treated with at least two prior treatment regimens.

110. The method of claim 109, wherein the prior treatment regimens did not comprise a multispecific antibody, e.g., a bispecific antibody.

111. The method of claim 109 or 110, wherein the prior treatment regimens included an immunomodulatory drug (IMiD), a proteasome inhibitor, an anti-CD38 inhibitor, or any combination thereof.

112. The method of any one of claims 109 to 111, wherein the prior treatment regimens included an IMiD that was lenalidomide, pomalidomide, or both.

113. The method of any one of claims 109 to 112, wherein the prior treatment regimens included a proteasome inhibitor that was bortezomib, carfilzomib, or both.

114. The method of any one of claims 109 to 113, wherein the prior treatment regimens included an anti-CD38 inhibitor that was an anti-CD38 antibody.

115. The method of claim 114, wherein the anti-CD38 antibody was daratumumab.

116. The method of any one of claims 109 to 115, wherein the prior treatment regimens included an autologous bone marrow transplant, a BCMA CAR-T, a BCMA antibody-drug conjugate, or any combination thereof.

117. The method of any one of claims 1 to 116, wherein the subject:
(a) does not have a history of severe hypersensitivity reactions to BSBM3;
(b) does not have a history of toxicity to prior BCMA targeted agents;
(c) does not have any other malignant disease other than cancer being treated and/or prevented;
(d) does not have any active, known or suspected autoimmune disease;
(e) is currently receiving treatment with a prohibited medication that cannot be discontinued at least one week prior to the start of this method;
(f) is not infected with human immunodeficiency virus (HIV), active hepatitis B virus (HBV), or hepatitis C virus (HCV);
(g) does not have impaired cardiac function or clinically significant cardiac disease including any of the following:
(i) clinically significant and/or uncontrolled heart disease such as congestive heart failure requiring treatment (NYHA Grade≥2), uncontrolled hypertension or clinically significant arrhythmia;
(ii) QTcF>470 msec on screening ECG or congenital long QT syndrome; or
(iii) acute myocardial infarction or unstable angina pectoris<3 months prior to study entry;
(h) has not had radiotherapy within 14 days before the first dose except for localized radiation therapy for lytic bone lesions or phasmacytomas;
(i) has not had a major surgery within 2 weeks before the first dose;
(j) has not used systemic chronic steroid therapy (≥10 mg/day of prednisone or equivalent), or any immunosuppressive therapy within 7 days of first dose;
(k) does not receive systemic treatment with any immunosuppressive medication;
(l) does not have Grade≥2 neuropathy, or residual toxic effects of from previous therapy that have not resolved to Grade≤1 or baseline;
(m) does not have plasma cell leukemia and other plasmacytoid disorders other than multiple myeloma;
(n) does not have any of the following clinical laboratory results:
(i) absolute neutrophil count (ANC)<1,000/mm3 without growth factor support within 7 days prior to the start of treatment;
(ii) platelet count<75,000 mm3 without transfusion support within 7 days prior to the start of treatment;
(iii) bilirubin>1.5 times the upper limit of the normal range (ULN);
(iv) aspartate aminotransferase (AST) or alanine aminotransferase (ALT)>3 times the ULN; or
(v) calculated creatinine clearance<30 ml/min according to Cockcroft-Gault equation;
(o) does not have an active infection requiring systemic therapy or other severe infection within 2 weeks before the first dose;
(p) does not have POEMS syndrome (plasma cell dyscrasia with polyneuropathy, organomegaly, endocrinopathy, monoclonal protein, skin changes);
(q) has not had prior allogeneic SCT at any time; or
(r) does not use of any live vaccines against infectious diseases (e.g. influenza, varicella, pneumococcus) within 4 weeks of the first dose;
(s) is not treated with cytotoxic or small molecule targeted antineoplastics, or any experimental therapy, within 14-days or 5 half-lives whichever is shorter before the first dose;
(t) has not had the initiation of hematopoietic colony-stimulating growth factors (e.g. G-CSF, M-CSF), thrombopoietin mimetics or erythroid stimulating agents≤2 weeks prior to start of treatment;
(u) has not had intravenous IG infusions for infection prophylaxis within the last 28 days prior to treatment;
(v) has not had active central nervous system (CNS) involvement by malignancy or presence of symptomatic CNS metastases, or CNS metastases that require local CNS-directed therapy (such as radiotherapy or surgery), or increasing doses of corticosteroids within the 2 weeks prior to the start of treatment;
(w) does not have serious medical or psychiatric illness likely to interfere with the treatment;
(x) if a woman, is not pregnant or nursing (lactating);
(y) if a woman of child-bearing potential (defined as a woman physiologically capable of becoming pregnant), is using two effective methods of contraception during dosing and for 6 months after the last dose of study drug, wherein at least one of the effective methods of contraception is a highly effective contraception method (e.g., i) total abstinence, ii) female sterilization, iii) male sterilization, or (iv) use of oral, injected or implanted hormonal methods of contraception or placement of an intrauterine device (IUD) or intrauterine system (IUS), or other form of hormonal contraception having a comparable efficacy (failure rate<1%), for example hormone vaginal ring or transdermal hormone contraception); or
(z) any combination thereof.

118. The method of any one of claims 1 to 117, wherein the administering of the bispecific antibody continues until the subject experiences toxicity, has clinical evidence of disease progression by IMWG, and/or treatment is discontinued at the discretion of the treating physician.

119. The method of any one of claims 1 to 118, wherein the bispecific antibody is administered in the form of a pharmaceutical composition comprising:
(a) the bispecific antibody;
(b) histidine;
(c) sucrose; and
(d) PS20.

120. The method of claim 119, wherein the pharmaceutical composition is in the form of a liquid.

121. The method of claim 119 or claim 120, wherein the histidine concentration in the pharmaceutical composition is about 20 mM.

122. The method of any one of claims 119 to 121, wherein the sucrose concentration in the pharmaceutical composition is about 240 mM.

123. The method of any one of claims 119 to 122, wherein the PS20 concentration in the pharmaceutical composition is about 0.04%.

124. The method of any one of claims 119 to 123, wherein the pH of the pharmaceutical composition is about 5.5±0.3.

125. A vial comprising:
(a) 10 mg/mL of a bispecific antibody that binds to human BCMA and human CD3 and comprises:
(i) a first polypeptide whose amino acid sequence comprises the amino acid sequence of SEQ ID NO:1;
(ii) a second polypeptide whose amino acid sequence comprises the amino acid sequence of SEQ ID NO:2; and
(iii) a third polypeptide whose amino acid sequence comprises the amino acid sequence of SEQ ID NO:3;
(b) 20 mM histidine;
(c) 240 mM sucrose;
(d) 0.04% PS20; and
(e) a pH of about 5.5±0.3.

126. A method of treating or preventing cancer comprising administering to the subject in need thereof, one or more treatment doses of a multispecific antibody having binding specificity towards at least B cell maturation antigen (BCMA) and a T-cell engaging arm, wherein the multispecific antibody, which is optionally BSBM3, is administered to the subject at a dose of:
(a) about 1 μg/kg to about 1000 μg/kg;
(b) about 0.25 μg/kg to about 1200 μg/kg;
(c) about 0.5 μg/kg to about 900 μg/kg; or
(d) about 1 μg/kg to about 600 μg/kg.

127. The method of claim 126, wherein the T-cell engaging arm binds to CD-3.

128. The method of claim 126 or claim 127, wherein the multispecific antibody is administered to the subject at a dose of about 0.5 μg/kg to about 900 μg/kg.

129. The method of any one of claims 126 to 127 wherein the multispecific antibody is administered to the subject at a dose of about 1 μg/kg to about 600 μg/kg.

130. The method of claim 127 or claim 129, wherein the multispecific antibody is administered to the subject at a dose of about 1 μg/kg.

131. The method of claim 127 or claim 129, wherein the multispecific antibody is administered to the subject at a dose of about 3 μg/kg.

132. The method of claim 127 or claim 129, wherein the multispecific antibody is administered to the subject at a dose of about 6 μg/kg.

133. The method of claim 127 or claim 129, wherein the multispecific antibody is administered to the subject at a dose of about 10 μg/kg.

134. The method of claim 127 or claim 129, wherein the multispecific antibody is administered to the subject at a dose of about 12 μg/kg.

135. The method of claim 127 or claim 129, wherein the multispecific antibody is administered to the subject at a dose of about 20 μg/kg to about 40 μg/kg.

136. The method of claim 127 or claim 129, wherein the multispecific antibody is administered to the subject at a dose of about 24 μg/kg

137. The method of claim 127 or claim 129, wherein the multispecific antibody is administered to the subject at a dose of about 30 μg/kg.

138. The method of claim 127 or claim 129, wherein the multispecific antibody is administered to the subject at a dose of about 40 μg/kg to about 80 μg/kg.

139. . The method of claim 127 or claim 129, wherein the multispecific antibody is administered to the subject at a dose of about 48 μg/kg.

140. The method of claim 127 or claim 129, wherein the multispecific antibody is administered to the subject at a dose of about 80 μg/kg to about 120 μg/kg.

141. The method of claim 127 or claim 129, wherein the multispecific antibody is administered to the subject at a dose of about 96 μg/kg.

142. The method of claim 127 or claim 129, wherein the multispecific antibody is administered to the subject at a dose of about 100 μg/kg.

143. The method of claim 127 or claim 129, wherein the multispecific antibody is administered to the subject at a dose of about 150 μg/kg to about 250 μg/kg.

144. The method of claim 127 or claim 129, wherein the multispecific antibody is administered to the subject at a dose of about 200 μg/kg.

145. The method of claim 127 or claim 129, wherein the multispecific antibody is administered to the subject at a dose of about 300 μg/kg to about 500 μg/kg.

146. The method of claim 127 or claim 129, wherein the multispecific antibody is administered to the subject at a dose of about 400 μg/kg.

147. The method of claim 127 or claim 129, wherein the multispecific antibody is administered to the subject at a dose of about 500 μg/kg to about 700 μg/kg.

148. The method of claim 127 or claim 129, wherein the multispecific antibody is administered to the subject at a dose of about 600 μg/kg.

149. The method of any one of claims 126 to 148, wherein the multispecific antibody is administered to the subject intravenously.

150. The method of any one of claims 126 to 149, wherein the multispecific antibody is administered over a 2 hour span.

151. The method of any one of claims 126 to 150, wherein the multispecific antibody is administered to the subject, once a week for 4 weeks.

152. The method of any one of claims 126 to 151, wherein the subject is administered a priming dose prior to administering the first treatment dose.

153. The method of claim 152, wherein administration of the priming dose is divided.

154. The method of claim 152 or 153, wherein the priming dose is given to the subject over two days.

155. The method of claim 154, wherein a third of the priming dose is given on the first day and two thirds of the priming dose is given on the second day.

156. The method of claim 154 or claim 155, wherein the two days are consecutive.

157. The method of any one of claims 152 to 156, wherein the priming dose is a lower dose than the treatment dose.

158. The method of any one of claims 127 to 157, wherein the subject is administered a side effect reducing agent.

159. The method of claim 158, wherein the side effect reducing agent reduces the onset or severity of cytokine release syndrome (CRS).

160. The method of claim 158 or 159, wherein the side effect reducing agent is a glucocorticoid.

161. The method of claim 160, wherein the glucocorticoid is methylprednisolone.

162. The method of claim 161, wherein the methylprednisolone is given at least 2 mg/kg.

163. The method of claim 158 or 159, wherein the side effect reducing agent is paracetamol, acetaminophen, antihistamines, steroids, anti-T cell directed therapy, or any combination thereof.

164. The method of claim 163, wherein the side effect reducing agent is an anti-T cell directed therapy that is tocilizumab, canakinumab, or any combination thereof.

165. The method of any one of claims 126 to 164, wherein the subject is administered a second therapeutic agent.

166. The method of claim 165, wherein the multispecific antibody and the second therapeutic agent are administered simultaneously, separately, or over a period of time.

167. The method of claim 165 or 166, wherein the second therapeutic agent is a gamma secretase inhibitor (GSI).

168. The method of claim 167, wherein the GSI is LY-450139, PF-5212362, BMS-708163, MK-0752, ELN-318463, BMS-299897, LY-411575, DAPT, AL-101 (BMS-906024), AL-102 (BMS-986115), PF-3084014, RO4929097, or LY3039478.

169. The method of claim 168, wherein the GSI is AL-102.

170. The method of any one of claims 167 to 169, wherein the GSI is administered orally.

171. The method of any one of claims 167 to 170, wherein the GSI is administered prior to administration of the multispecific antibody.

172. The method of claim 165 or 166, wherein the second therapeutic agent is an immunomodulator.

173. The method of claim 165 or 166, wherein the second therapeutic agent is an immune checkpoint inhibitor.

174. The method of claim 165 or 166 wherein the second therapeutic agent is a TIM-3 inhibitor.

175. The method of claim 174, wherein the TIM-3 inhibitor is MBG453.

176. The method of claim 165 or 166, wherein the second therapeutic agent is a LAG-3 inhibitor.

177. The method of claim 176, wherein the LAG-3 inhibitor is LAG525.

178. The method of claim 165 or 166, wherein the second therapeutic agent is a PD-1 inhibitor.

179. The method of claim 178, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

180. The method of claim 178 or 179, wherein the PD-1 inhibitor is PDR001.

181. The method of claim 179 or 180, wherein the PD-1 inhibitor is administered at a dose of about 100 mg once every four weeks, or about 200 mg once every four weeks, or about 300 mg once every four weeks, or about 400 mg once every four weeks, or about 500 mg once every four weeks.

182. The method of claim 181, wherein the PD-1 inhibitor is administered at a dose of about 400 mg once every four weeks.

183. The method of any one of claims 165 to 182, wherein the second therapeutic agent is administered intravenously.

184. The method of any one of claims 126 to 183, wherein the cancer is a blood cancer.

185. The method of claim 184, wherein the blood cancer is multiple myeloma.

186. The method of claim 184 or 185, wherein the subject has relapsed and/or refractory multiple myeloma.

187. The method of any one of claims 126 to 186, wherein the subject has been previous treated with at least two prior treatment regimens.

188. The method of claim 187, wherein the prior treatment regimens did not comprise a multispecific antibody.

189. The method of claim 187 or 188, wherein the prior treatment regimens included an immunomodulatory drug (IMiD), a proteasome inhibitor, an anti-CD38 inhibitor, or any combination thereof.

190. The method of any one of claims 187 to 189, wherein the prior treatment regimens included an IMiD that was lenalidomide, pomalidomide, or both.

191. The method of any one of claims 187 to 190, wherein the prior treatment regimens included a proteasome inhibitor that was bortezomib, carfilzomib, or both.

192. The method of any one of claims 187 to 191, wherein the prior treatment regimens included an anti-CD38 inhibitor that was an anti-CD38 antibody.

193. The method of claim 192, wherein the anti-CD38 antibody was daratumumab.

194. The method of any one of claims 187 to 193, wherein the prior treatment regimens included an autologous bone marrow transplant, a BCMA CAR-T, a BCMA antibody-drug conjugate, or any combination thereof.

195. The method of any one of claims 126 to 194, wherein the subject has:
(a) a serum M-protein greater than equal to 1.0 g/dL;
(b) a urine M-protein greater than equal to 200 mg/24 hours;
(c) a serum free light chain (sFLC) greater than 100 mg/L of involved FLC; or
(d) any combination thereof.

196. The method of any one of claims 126 to 195, wherein the subject:
(a) does not have a history of severe hypersensitivity reactions to the multispecific antibody;
(b) does not have a history of toxicity to prior BCMA targeted agents;
(c) does not have any other malignant disease other than cancer being treated and/or prevented;
(d) does not have any active, known or suspected autoimmune disease;
(e) is currently receiving treatment with a prohibited medication that cannot be discontinued at least one week prior to the start of this method;
(f) is not infected with human immunodeficiency virus (HIV), active hepatitis B virus (HBV), or hepatitis C virus (HCV);
(g) does not have impaired cardiac function or clinically significant cardiac disease including any of the following:
(i) clinically significant and/or uncontrolled heart disease such as congestive heart failure requiring treatment (NYHA Grade≥2), uncontrolled hypertension or clinically significant arrhythmia;
(ii) QTcF>470 msec on screening ECG or congenital long QT syndrome; or
(iii) acute myocardial infarction or unstable angina pectoris<3 months prior to study entry;
(h) has not had radiotherapy within 14 days before the first dose except for localized radiation therapy for lytic bone lesions or phasmacytomas;
(i) has not had a major surgery within 2 weeks before the first dose;
(j) has not used systemic chronic steroid therapy (≥10 mg/day of prednisone or equivalent), or any immunosuppressive therapy within 7 days of first dose;
(k) does not receive systemic treatment with any immunosuppressive medication;
(l) does not have Grade≥3 neuropathy, or residual toxic effects of Grade≥2 from previous therapy;
(m) does not have plasma cell leukemia and other plasmacytoid disorders other than multiple myeloma;
(n) does not have any of the following clinical laboratory results:
(i) absolute neutrophil count (ANC)<1,000/mm3 without growth factor support within 7 days prior to the start of treatment;
(ii) platelet count<75,000 mm3 without transfusion support within 7 days prior to the start of treatment;
(iii) bilirubin>1.5 times the upper limit of the normal range (ULN);
(iv) aspartate aminotransferase (AST) or alanine aminotransferase (ALT)>3 times the ULN; or
(v) calculated creatinine clearance<30 ml/min according to Cockcroft-Gault equation;
(o) does not have an active infection requiring systemic therapy or other severe infection within 2 weeks before the first dose;
(p) does not have POEMS syndrome (plasma cell dyscrasia with polyneuropathy, organomegaly, endocrinopathy, monoclonal protein, skin changes);
(q) has not had prior allogeneic SCT at any time; or
(r) does not use of any live vaccines against infectious diseases (e.g. influenza, varicella, pneumococcus) within 4 weeks of the first dose;
(s) is not treated with cytotoxic or small molecule targeted antineoplastics, or any experimental therapy, within 14-days or 5 half-lives whichever is shorter before the first dose;
(t) has not had the initiation of hematopoietic colony-stimulating growth factors (e.g. G-CSF, M-CSF), thrombopoietin mimetics or erythroid stimulating agents≤2 weeks prior to start of treatment;
(u) has not had intravenous IG infusions for infection prophylaxis within the last 28 days prior to treatment;
(v) has not had active central nervous system (CNS) involvement by malignancy or presence of symptomatic CNS metastases, or CNS metastases that require local CNS-directed therapy (such as radiotherapy or surgery), or increasing doses of corticosteroids within the 2 weeks prior to the start of treatment;
(w) does not have serious medical or psychiatric illness likely to interfere with the treatment;
(x) is not pregnant or nursing (lactating) women;
(y) women of child-bearing potential (defined as all women physiologically capable of becoming pregnant) unless they are using highly effective methods of contraception during dosing and for 90 days after the last dose of study drug, wherein the highly effective contraception methods including i) total abstinence, ii) female sterilization, iii) male sterilization, or (iv) use of oral, injected or implanted hormonal methods of contraception or placement of an intrauterine device (IUD) or intrauterine system (IUS), or other forms of hormonal contraception that have comparable efficacy (failure rate<1%), for example hormone vaginal ring or transdermal hormone contraception; or
(z) any combination thereof.

197. The method of any one of claims 126 to 196, wherein the multispecific antibody is a bispecific antibody.

198. The method of claim 197, wherein the bispecific antibody specifically binds to human BCMA and comprises a first polypeptide whose amino acid sequence comprises the amino acid sequence of SEQ ID NO:1; a second polypeptide whose amino acid sequence comprises the amino acid sequence of SEQ ID NO:2; and a third polypeptide whose amino acid sequence comprises the amino acid sequence of SEQ ID NO:3.

199. The method of any one of claims 126 to 198, wherein the administering of the multispecific antibody continues until the subject experiences toxicity, has clinical evidence of disease progression by IMWG, and/or treatment is discontinued at the discretion of the treating physician.

200. A combination therapy comprising a multispecific antibody having binding specificity towards at least B cell maturation antigen (BCMA) and a T-cell engaging arm and a second therapeutic agent.

201. The combination therapy of claim 200, wherein the second therapeutic agent is a gamma secretase inhibitor (GSI).

202. The combination therapy of claim 201, wherein the GSI is LY-450139, PF-5212362, BMS-708163, MK-0752, ELN-318463, BMS-299897, LY-411575, DAPT, AL-101 (BMS-906024), AL-102 (BMS-986115), PF-3084014, RO4929097, or LY3039478.

203. The combination therapy of claim 202, wherein the GSI is AL-102.

204. The combination therapy of claim 200, wherein the second therapeutic agent is an immunomodulator.

205. The combination therapy of claim 200, wherein the second therapeutic agent is an immune checkpoint inhibitor.

206. The combination therapy of claim 200, wherein the second therapeutic agent is a TIM-3 inhibitor.

207. The combination therapy of claim 206, wherein the TIM-3 inhibitor is MBG453.

208. The combination therapy of claim 200, wherein the second therapeutic agent is a LAG-3 inhibitor.

209. The combination therapy of claim 208, wherein the LAG-3 inhibitor is LAG525.

210. The combination therapy of claim 200, wherein the second therapeutic agent is a PD-1 inhibitor.

211. The combination therapy of claim 210, wherein the PD-1 inhibitor is PDR001, Nivolumab, Pembrolizumab, Pidilizumab, MEDI0680, REGN2810, TSR-042, PF-06801591, BGB-A317, BGB-108, INCSHR1210, or AMP-224.

212. The combination therapy of any one of claims 200 to 211, wherein the combination comprises about 100 mg, or about 200 mg, or about 300 mg, or about 400 mg, or about 500 mg of the second therapeutic agent.

213. The combination therapy of any one of claims 200 to 211, wherein the combination comprises about 2 mg, or about 10 mg, or about 20 mg, or about 40 mg, or about 80 mg, or about 160 mg, or about 320 mg of the compound; and about 100 mg, or about 200 mg, or about 300 mg, or about 400 mg, or about 500 mg of the second therapeutic agent.

214. A combination therapy of any one of claims 200 to 213 for use in the treatment of cancer.

215. A combination therapy of any one of claims 200 to 214 for use in the prevention of cancer.

216. The combination therapy of claim 214 or claim 215 for administration according to the method of any one of claims 90 to 124 or 165 to 197.

217. Use of the combination therapy of any one of claims 200 to 216 for the manufacture of a medicament for treating or preventing cancer.

218. Use of the combination therapy of any one of claims 200 to 216 for the treatment of cancer.

219. Use of the combination therapy of any one of claims 200 to 216 for the prevention of cancer.

220. The combination therapy of any one of claims 200 to 216 or the use of claims 217 to 219, wherein the cancer is a blood cancer.

221. The combination therapy or use of claim 220, wherein the blood cancer is multiple myeloma.

222. A pharmaceutical composition comprising
(a) multispecific antibody having binding specificity towards at least B cell maturation antigen (BCMA) and a T-cell engaging arm;
(b) histidine;
(c) sucrose; and
(d) PS20.

223. The pharmaceutical composition of claim 222, wherein the composition is a liquid.

224. The pharmaceutical composition of claim 222 or 223, wherein the histidine concentration is 20 mM.

225. The pharmaceutical composition of any one of claims 222 to 224, wherein the sucrose concentration is 240 mM.

226. The pharmaceutical composition of any one of claims 222 to 225, wherein the PS20 concentration is 0.04%.

227. The pharmaceutical composition of any one of claims 222 to 226, wherein the pH is about 5.5±0.3.

228. A vial comprising:
(a) 10 mg/mL of a multispecific antibody having binding specificity towards at least B cell maturation antigen (BCMA) and a T-cell engaging arm
(b) 20 mM histidine;
(c) 240 mM sucrose;
(d) 0.04% PS20; and
(e) a pH of about 5.5±0.3.


SEQUENCE LISTING (6 sequences)

SEQ ID NO: 1 - protein, 452 aa, Artificial Sequence
  Synthetic polypeptide
QVQLVESGGGVVQPGRSLRLSCAASGFTVSSYGMHWVRQAPGKGLEWVAVISYTGSNKYYADSVKGRFTISRDNSKNTLYLQMNSLRAEDTAVYYCGGSGYALHDDYYGLDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 2 - protein, 217 aa, Artificial Sequence
  Synthetic polypeptide
QSALTQPASVSGSPGQSITISCTGTSSDVGGYNYVSWYQQHPGKAPKLMIYDVSNRLRGVSNRFSGSKSGNTASLTISGLQAEDEADYYCSSYTSSSALYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 485 aa, Artificial Sequence
  Synthetic polypeptide
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1356 bp, Artificial Sequence
  Synthetic polynucleotide
caagtgcagctcgtggagtctggagggggagtcgtgcagcctggacgctccctgagactgtcctgtgcggcttcgggattcactgtgtccagctacggcatgcattgggtccgccaagcaccgggaaaaggcctggagtgggtggccgtgatctcctacaccggctcaaacaagtactacgccgacagcgtgaagggccggttcaccatttcaagggacaactccaagaataccctgtatctgcaaatgaactcgctgcgggcagaggacaccgccgtgtactactgcggtggctccggttacgccctgcacgatgactactacgggctcgatgtctggggacaggggacgctcgtgactgtgtcctcggctagcaccaagggcccgtcagtgtttcctctggccccaagctccaagtccacctccggtggtacagccgcgttgggatgcttggtcaaggactactttccggaacccgtgaccgtgtcctggaactccggcgccctgactagcggagtgcacaccttccccgctgtgctgcagtctagcgggctgtattccctctcgtccgtggtcaccgtgccgtcctcatccctgggaacccagacctacatttgcaacgtgaaccacaagccgtcagacaccaaggtggacaagaaggtggagccgaagtcctgcgacaagacccatacttgtcctccttgccccgctccacctgtggcgggaccttccgtgttccttttcccgccgaagccgaaggacactctgatgatctcgcggactcccgaagtcacttgcgtggtggtggacgtcaaacacgaagatcccgaggtcaagttcaattggtacgtggacggggtggaagtccacaacgccaagactaagccgcgcgaggaagagtacaattccacttaccgggtcgtgtcggtgctgactgtgctgcatcaggactggctgaacggaaaggagtacaagtgcaaagtgtcgaacaaggccctgcctgcaccaatcgaaaagaccattagcaaagccaagggccagccgagagaaccccaagtctacactctgccaccatcccgcgaagaaatgaccaagaaccaagtgtcgctgacgtgcgacgtgtcgggattctacccgtccgatattgccgtggaatgggagagcgacggccaacccgagaacaactacaagactaccccccccgtcttggattccgatggttccttcttcctgtactccaagctgaccgtggataagtcccgatgggagcagggcgatgtgttctcgtgctccgtgatgcatgaagccctgcacaaccactatacccagaagtcactgtcgctgagccctgggaag

SEQ ID NO: 5 - DNA, 651 bp, Artificial Sequence
  Synthetic polynucleotide
cagtcggcgctgactcagcccgcatccgtgagcggttcaccgggacagagcatcaccatttcctgcaccggaacctcaagcgacgtgggcggctacaactacgtgtcctggtatcagcagcacccgggaaaggccccaaagctcatgatctacgacgtgtccaatagactgcggggagtgtccaaccggttctcgggaagcaaatccggcaacactgcttccctgaccatcagcggactccaggccgaagatgaggccgactactactgctcatcctacacgtcctcttcggcgctttacgtgttcgggtcggggaccaaggtcaccgtcctgggccaacctaaggcggcgccctcagtgaccctgttccctccgtcgtctgaagaactccaggccaacaaggccaccctcgtgtgcctgatttcggacttctacccgggagccgtcactgtggcctggaaggccgacagcagcccagtgaaggccggcgtggaaactaccaccccgtccaagcagtccaacaataagtacgcagccagctcctacctgtccctgacccccgaacaatggaagtcacacagatcctactcctgtcaagtcacccacgagggcagcactgtcgaaaagaccgtggcaccgactgagtgctcg

SEQ ID NO: 6 - DNA, 1455 bp, Artificial Sequence
  Synthetic polynucleotide
gaagtgcagcttgtggagtccgggggaggattggtccaacccggtggctcgctgaggctgagttgcgccgcttcggggtttaccttcagcacctacgctatgaactgggtcagacaggcgcctggaaagggtttggagtgggtcggacgcatccggtccaaggccaacaactacgcgacttactatgccgactccgtcaagggacggttcaccatctcccgggacgacagcaagaacaccctgtacctccaaatgaactcccttcgggccgaagataccgccgtgtactactgcgtgagacacggcaacttcggcgactcctacgtgtcctggtttgcctactggggccagggtactctcgtgaccgtgtcatcaggaaagccaggctcggggaagcctggctccggaaagcctgggagcggaaagccgggatcgcaggctgtggtcacccaggaaccctccctgactgtgtccccgggaggaaccgtgacactgacttgtggcagctccaccggagccgtgaccacctccaactacgccaactgggtgcagcaaaagccaggaaagtcccctagggggctgatcggtggcacgaacaagcgggcacctggagtgcctgcccgattctcgggtagcctgctggggggaaaagccgccctgaccatttcgggcgctcagccagaggacgaagccgactattactgcgcactctggtactccaaccactgggtgttcggtggaggcaccaagctgaccgtgctggagccaaagtcaagcgacaaaactcacacttgccctccttgtccggctcctcctgtggctggtccctccgtgttcctcttcccgccgaagccgaaggacaccctcatgatttcccggacgcccgaagtcacttgtgtggtggtcgatgtgaagcatgaggaccccgaagtgaagttcaattggtacgtggatggcgtggaggtccacaacgccaagaccaagccgcgcgaagaacagtacaacagcacctaccgcgtcgtgagcgtgctcaccgtgctccaccaagattggctgaacggaaaggagtacaagtgcaaagtgtccaacaaggcccttcctgcacctattgaaaagactattagcaaggccaagggacagccccgcgaacctcaagtgtacactctgccgccgtccagagagcagatgaccaaaaaccaggtcaagctcacttgtctcgtgaagggcttctacccgtccgatatcgcggtcgaatgggagtcaaacggccagcccgagaacaactacaagactaccccaccggtgcttgactccgacggttcgttctttctgtactccaagctgaccgtggacaagtcccggtggcagcaagggaatgtgttcagctgctccgtgatgcacgaagccctgcataaccactacacccagaagtcgctcagcctgtcccctggaaaa